(19) United States
(12) Patent Application Publication
(10) Pub. No.: US 2023/0044997 A1
(43) Pub. Date: Feb. 9, 2023

(54) SYNTHETIC, PERSISTENT RNA CONSTRUCTS AND METHODS OF USE FOR CELL REJUVENATION AND FOR TREATMENT

(71) Applicant: TURN BIOTECHNOLOGIES, INC., Mountain View, CA (US)

(72) Inventors: Naveen BOJJIREDDY, Milpitas, CA (US); Tapash Jay SARKAR, Tomball, TX (US)

(21) Appl. No.: 17/812,711

(22) Filed: Jul. 14, 2022

Related U.S. Application Data
(60) Provisional application No. 63/222,300, filed on Jul. 15, 2021.

(51) Int. Cl. C12N 15/11; C12N 15/86

(52) U.S. Cl. CPC C12N 15/11; C12N 15/86; C12N 2770/36143; C12N 2501/60

(57) ABSTRACT

Methods for treating a cell, tissue, or organ and for treating an age-related disease or condition are provided, where the cell, tissue or organ is contacted with a synthetic, persistent RNA vector comprising one or more heterologous polynucleotide sequences, each of the one or more heterologous polynucleotide sequences encoding for a reprogramming factor. Contacting achieves expression of the one or more reprogramming factors in the cell, tissue, or organ to treat the age-related disease or condition. In an embodiment, the method is used to obtain a rejuvenated cell, tissue, or organ with retention of cellular identity.

### CROSS-REFERENCE TO RELATED APPLICATIONS

This application claims the benefit of U.S. Provisional Application No. 63/222,300, filed Jul. 15, 2021, which is incorporated by reference herein.

### REFERENCE TO SEQUENCE LISTING, TABLE OR COMPUTER PROGRAM

A “Sequence Listing is submitted with this application in the form of a text file, created Jul. 12, 2022, and named “111277-0043-8002US00_SEQ” (35,000 bytes), the contents of which are incorporated herein by reference in their entirety. Peptide sequences related to the present disclosure are also provided in Table 1.

### TECHNICAL FIELD

The subject matter described herein relates to compositions and methods for cellular rejuvenation, tissue engineering, regenerative medicine and disease treatment, by transient exposure of cells or tissues to synthetic, persistent expression constructs that comprise polyribonucleotides encoding one or more reprogramming factors.

### BACKGROUND

Aging is characterized by a gradual loss of function occurring at the molecular, cellular, tissue and organismal levels. At the chromatin level, aging is associated with the progressive accumulation of epigenetic errors that eventually lead to aberrant gene regulation, stem cell exhaustion, senescence, and deregulated cell/tissue homeostasis. The technology of nuclear reprogramming to pluripotency, through over-expression of a small number of transcription factors, can revert both the age and the identity of any cell to that of an embryonic cell by driving epigenetic reprogramming. This reversion of cellular age is beneficial in age-related conditions and disease, such as cancer, and is also beneficial in rejuvenative therapies. In the latter, the undesirable erasure of cell identity is problematical for the development of rejuvenative therapies because of the resulting destruction of the structure, function and cell type distribution in tissues and organs. There is a need for methods of rejuvenating cells where dedifferentiation and loss of cell identity can be controllable avoided. The present disclosure addresses this need, and provides additional benefits as well.

### BRIEF SUMMARY

The following aspects and embodiments thereof described and illustrated below are meant to be exemplary and illustrative, not limiting in scope.

In one aspect, a method of treating a cell, tissue or organ in a subject in need thereof is provided. The method comprises contacting the cell, tissue or organ with a synthetic, persistent RNA vector comprising one or more heterologous polynucleotide sequences, each of the one or more heterologous polynucleotide sequences encoding for a reprogramming factor, whereby the contacting achieves expression of the one or more reprogramming factors in the cell, tissue or organ.

In an embodiment, expression of the one or more reprogramming factors is for a defined period of time. In an embodiment, the defined period of time is determined by a mechanism in the vector that silences the expression. This embodiment wherein the vector optionally comprises a silencing mechanism is useful for certain methods of treatment, such as methods involved with cell rejuvenation with retention of cellular identity. Silencing, ceasing or curtailing expression of the one or more reprogramming factors permits generation of a rejuvenated cell, tissue or organ with retention of cellular identity.

In an embodiment, the mechanism to silence expression is a mechanism capable of and/or configured to control expression by silencing expression in response to one or more triggers and initiating expression in response to one or more triggers. The mechanism in the vector is, in an embodiment, configured as an on/off switch of expression of the one or more reprogramming factors.

In an embodiment, the mechanism to silence, curtail or cease expression and/or to initiate or turn ‘on’ expression of the one or more reprogramming factors of a persistent, self-amplifying vector is a modification to the sequence of the RNA-dependent RNA polymerase (RdRp) complex. The modification to the RNA-dependent RNA polymerase sequence is, for example, configured to provide controlled synthesis and construction of the RdRp complex or to provide controlled degradation of the RdRp complex in order to control mRNA amplification. In another embodiment, the mechanism to silence, curtail or cease expression of the one or more reprogramming factors of a persistent, self-amplifying vector is selected from a modification of the sub-genomic promoter to control gene(s) of interest expression, a modification of the auxiliary mRNA stability elements (e.g., cap, tail, UTRs, etc.) to control mRNA lifetime, sequence tailoring for degradation by selective endo/exo-nucleases to degrade mRNA, or a sequence modification to control the general cellular response to synthetic mRNAs, such as use of a molecule like B18R (soluble interferon alpha/beta receptor B18), B19R, or other decoy molecules, or the use of select media or environmental stimulators.

In an embodiment, the mechanism to silence, curtail or cease expression and/or to initiate or turn ‘on’ expression of the one or more reprogramming factors of a persistent, circular or endless RNA vector is sequence tailoring for degradation by selective endonucleases to degrade mRNA, such as, for example, RNase L. In another embodiment, the mechanism is selected from a modification to the internal ribosomal entry site or the m6A site to control translation; the use of stop codons to introduce translational cessation; a modification of the sub genomic promoter to control gene(s) of interest expression; or control of the general cellular response to synthetic mRNAs.

In another aspect, a method for treating a differentiated cell is provided. The method comprises introducing a synthetic, persistent RNA vector encoding one or more reprogramming factors into the differentiated cell for expression of the one or more reprogramming factors. In an embodiment, the synthetic, persistent RNA vector comprises a mechanism to silence, curtail or cease expression of the one or more reprogramming factors, and optionally, to initiate or turn ‘on’ expression of the one or more reprogramming factors, to thereby generate a cell that retains its cellular differentiation and that expresses the one or more reprogramming factor to obtain a rejuvenated cell.

In embodiments, the cell does not become an induced pluripotent stem cell. It retains its cellular identity and enters a rejuvenated condition by on/off expression of the one or more reprogramming factors.

In one embodiment, the vector is a polycistronic vector comprising two or more or three or more reprogramming factors.

In some embodiments, the RNA vectors provided herein, comprise a reprogramming factors such as Oct, Sox, Klf, Lin, Nanog, Glis or Myc. In some embodiments, the reprogramming factor is OCT4, SOX2, KLF4, LIN28, NANOG, c-Myc, or GLIS1.

A method of treating an age-related disease or condition is provided. The method comprises exposing differentiated cells associated with the age-related disease or condition to a synthetic, persistent RNA vector encoding one or more reprogramming factors. In an embodiment, the synthetic, persistent RNA vector comprises a mechanism to silence, curtail or cease expression of the one or more reprogramming factors, and optionally, to initiate or turn ‘on’ expression of the one or more reprogramming factors, to thereby generate a cell that retains its cellular differentiation and that expresses the one or more reprogramming factor to obtain a rejuvenated cell. The exposing achieves expression of the one or more reprogramming factors in the differentiated cells to obtain rejuvenated cells with retention of cellular identity.

In embodiments, the synthetic, persistent RNA vector is a self-replicating vector or a circular polyribonucleotide.

In some embodiments, the reprogramming factor comprises OCT4, wherein the OCT4 consists of a nucleotide sequence having at least 95% sequence identity to SEQ ID NO: 1. In some embodiments, the reprogramming factor comprises SOX2, wherein the SOX2 consists of a nucleotide sequence having at least 95% sequence identity to SEQ ID NO: 2. In some embodiments, the reprogramming factor comprises c-Myc, wherein the c-Myc consists of a nucleotide sequence having at least 95% sequence identity to SEQ ID NO: 3. In some embodiments, the reprogramming factor comprises KLF4, wherein the KLF4 consists of a nucleotide sequence having at least 95% sequence identity to SEQ ID NO: 4. In some embodiments, the reprogramming factor comprises LIN28, wherein the LIN28 consists of a nucleotide sequence having at least 95% sequence identity to SEQ ID NO: 1. In some embodiments, the reprogramming factor comprises NANOG, wherein the NANOG consists of a nucleotide sequence having at least 95% sequence identity to SEQ ID NO: 6. In some embodiments, the reprogramming factor comprises GLIS1 wherein GLIS1 consists of a nucleotide sequence having at least 95% sequence identity to SEQ ID NO: 10.

In some embodiments, the RNA vector comprises a first polynucleotide sequence of an OCT4 nucleotide sequence having at least 95% sequence identity to SEQ ID NO: 1, a second polynucleotide sequence of a SOX2 nucleotide sequence having at least 95% sequence identity to SEQ ID NO: 2, and a third polynucleotide sequence of an KLF4 nucleotide sequence having at least 95% sequence identity to SEQ ID NO: 4.

In some embodiments, the RNA vector comprises a first polynucleotide sequence of an LIN28 sequence having at least 95% sequence identity to SEQ ID NO: 5, a second polynucleotide sequence of an NANOG sequence having at least 95% sequence identity to SEQ ID NO: 6, and a third polynucleotide sequence of an c-Myc sequence having at least 95% sequence identity to SEQ ID NO: 3.

In some embodiments, the RNA vector comprises a first polynucleotide sequence, a second polynucleotide sequence and a third polynucleotide sequence, each independently selected from the group consisting of nucleotides comprising at least 90%, 91%, 92%, 93%, 94%, 95%, 96%, 97%, 98% or 99% sequence identity to any one of SEQ ID NOs: 1-6.

In some embodiments, the RNA vectors provided herein are transcription vectors comprising a transcription initiation region. In some embodiments, the RNA vectors include a poly A tail. In other embodiments, the RNA vectors include tails that comprise a heteropolymer insert, such as a tail having at least about 80%, about 85%, about 90%, about 95%, about 96%, about 97%, about 98%, about 99%, about 100% sequence identity to SEQ ID NO: 7 and/or comprising, consisting essentially of or consisting of SEQ ID NO: 7. In some embodiments, the RNA vectors comprise untranslated regions (UTRs), such as a 5′UTR and/or a 3′ UTR. In some embodiments, the RNA vectors include a 5′ UTR that has at least about 80%, about 85%, about 90%, about 95%, about 96%, about 97%, about 98%, about 99%, about 100% sequence identity to SEQ ID NO: 8 and/or comprising, consisting essentially of or consisting of SEQ ID NO: 8. In some embodiments, the RNA vectors include a 3′ UTR that has at least about 80%, about 85%, about 90%, about 95%, about 96%, about 97%, about 98%, about 99%, about 100% sequence identity to SEQ ID NO: 9 and/or comprising, consisting essentially of or consisting of SEQ ID NO: 9. In some embodiments, the RNA vectors may also include linker regions, and/or cap regions. In some embodiments, the RNA vectors provided herein comprise at least one modified base pair, such as an N1-methyl-pseudo-uridine-triphosphate.

In addition to the exemplary aspects and embodiments described above, further aspects and embodiments will become apparent by reference to the drawings and by study of the following descriptions.

Additional embodiments of the present methods and compositions, and the like, will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present disclosure. Additional aspects and advantages of the present disclosure are set forth in the following description and claims, particularly when considered in conjunction with the accompanying examples and drawings.

### BRIEF DESCRIPTION OF THE DRAWINGS

FIG. 1 illustrates a procedure to prepare a circular mRNA, in one embodiment.

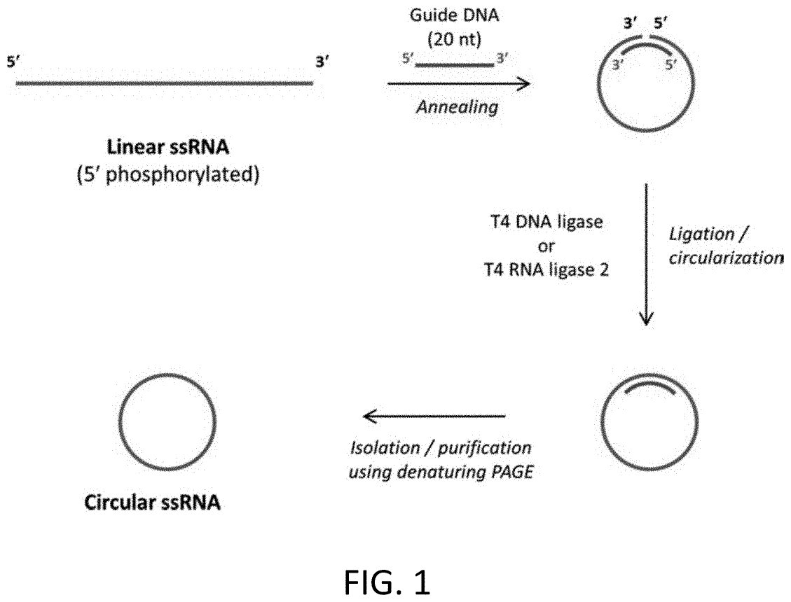

### BRIEF DESCRIPTION OF THE SEQUENCES

In some embodiments, the methods and compositions for cellular rejuvenation, tissue engineering, and regenerative medicine by transient exposure of cells or tissues to synthetic, non-integrative mRNAs encoding reprogramming factors, comprise exposing the immune cell to messenger RNA (mRNA) encoding one or more reprogramming factors wherein the reprogramming factor encoding mRNA encodes a polypeptide encoded by a polynucleotide having at least 85%, 90%, 91%, 92%, 93%, 94%, 95%, 96%, 97%, 98%, 99%, or 100% sequence identity to any one of SEQ ID NOs: 1-19 (Table 1).

TABLE 1

| | | SEQ |
|---|---|---|
| NAME | SEQUENCE | ID NO |
| OCT4 | ATGGCTGGCCATCTCGCAAGTGACTTCGCATTTTCCCCG | 1 |
| | CCCCCAGGCGGCGGTGGAGATGGACCTGGCGGCCCAGA | |
| | ACCAGGATGGGTGGACCCCCGCACGTGGCTTTCTTTTCA | |
| | GGGTCCACCAGGCGGACCCGGTATTGGACCCGGCGTGG | |
| | GTCCTGGGTCAGAAGTCTGGGGTATCCCACCCTGTCCCC | |
| | CACCATACGAATTTTGCGGCGGAATGGCCTATTGCGGCC | |
| | CTCAAGTCGGGGTCGGTCTGGTACCTCAGGGTGGTCTGG | |
| | AAACATCCCAACCAGAAGGTGAGGCCGGTGTGGGAGTT | |
| | GAATCCAATTCTGACGGAGCATCTCCAGAACCTTGTACT | |
| | GTGACACCAGGAGCTGTTAAATTGGAGAAAGAAAAGCT | |
| | CGAACAGAATCCAGAAGAATCACAAGATATTAAGGCGC | |
| | TCCAAAAGGAGCTGGAACAGTTCGCGAAACTTCTTAAAC | |
| | AGAAACGCATTACTCTCGGGTACACCCAAGCGGACGTTG | |
| | GACTGACTCTCGGTGTGCTGTTCGGCAAAGTCTTTAGTC | |
| | AGACCACAATATGTCGATTCGAAGCCCTTCAACTGTCAT | |
| | TTAAGAATATGTGCAAACTTCGACCTCTGCTCCAGAAAT | |
| | GGGTCGAAGAGGCGGATAATAACGAGAACCTGCAAGAA | |
| | ATCTGTAAGGCGGAGACTCTGGTTCAAGCTCGCAAAAGG | |
| | AAACGTACGTCTATAGAAAATAGAGTCCGTGGGAATCTT | |
| | GAAAACCTGTTTCTCCAATGTCCAAAGCCTACTTTGCAA | |
| | CAAATATCTCATATTGCGCAACAACTCGGCCTGGAAAAG | |
| | GACGTAGTTAGAGTCTGGTTTTGCAATCGCAGACAGAAA | |
| | GGGAAACGGTCTTCCAGTGATTACGCGCAGAGGGAAGA | |
| | CTTCGAAGCAGCCGGTTCACCGTTTTCCGGCGGCCCGGT | |
| | ATCTTTCCCATTGGCTCCCGGTCCTCACTTCGGCACACCC | |
| | GGGTACGGCTCACCACATTTTACCGCCCTTTATTCAAGC | |
| | GTTCCCTTTCCGGAAGGCGAGGCTTTCCCGCCGGTGTCA | |
| | GTGACTACACTTGGATCCCCAATGCACAGCAATTAG | |
| SOX2 | ATGTATAATATGATGGAAACCGAATTGAAACCACCCGG | 2 |
| | GCCCCAACAGACCTCTGGCGGCGGTGGTGGTAATAGCAC | |
| | AGCAGCAGCCGCTGGTGGAAATCAAAAGAATTCTCCAG | |
| | ATAGAGTGAAACGACCTATGAACGCATTTATGGTCTGGT | |
| | CTAGAGGACAACGAAGGAAAATGGCTCAAGAAAATCCC | |
| | AAAATGCATAATAGCGAAATTTCCAAACGGTTGGGTGCG | |
| | GAATGGAAGCTCCTCAGCGAAACCGAAAAGAGGCCATT | |
| | TATTGATGAAGCGAAAAGACTCAGGGCATTGCATATGA | |
| | AAGAACATCCAGACTACAAGTATAGACCACGCCGCAAG | |
| | ACAAAGACTCTGATGAAGAAGGACAAATATACCCTGCC | |
| | TGGTGGATTGTTGGCTCCTGGCGGTAACAGTATGGCTTC | |
| | TGGCGTGGGCGTTGGGGCTGGACTTGGTGCCGGGGTCAA | |
| | TCAACGAATGGATTCCTATGCCCATATGAATGGATGGAG | |
| | TAATGGTTCCTATTCTATGATGCAAGATCAATTGGGATA | |
| | TCCTCAACATCCCGGTCTGAACGCTCATGGTGCTGCTCA | |
| | AATGCAACCTATGCATCGGTATGATGTAAGTGCATTGCA | |
| | ATATAATAGCATGACATCCAGTCAAACATATATGAATGG | |
| | GTCACCAACATATAGTATGAGCTATTCCCAACAAGGTAC | |
| | ACCAGGGATGGCCCTGGGGAGCATGGGGAGTGTCGTTA | |
| | AAAGTGAAGCTTCAAGTTCACCACCCGTAGTGACGAGTT | |
| | CTTCACATTCTCGAGCCCCATGTCAAGCAGGAGATCTTA | |
| | GGGATATGATTTCAATGTACTTGCCAGGGGCTGAAGTCC | |
| | CCGAGCCGGCAGCGCCTTCTAGGCTGCATATGTCTCAAC | |
| | ATTATCAATCCGGACCCGTTCCAGGGACCGCTATCAATG | |
| | GTACGTTGCCATTGTCCCATATGTAA | |
| C-Myc | ATGGACTTCTTTCGAGTGGTAGAGAATCAACAACCCCCG | 3 |
| | GCCACCATGCCATTGAATGTATCATTTACAAACCGGAAC | |
| | TACGACCTGGACTATGATTCAGTTCAGCCTTACTTTTATT | |
| | GTGACGAAGAAGAAAATTTCTATCAACAACAACAACAA | |
| | TCAGAATTGCAACCACCCGCTCCATCAGAAGACATTTGG | |
| | AAGAAGTTTGAACTCCTGCCAACTCCACCGCTCAGCCCG | |
| | AGTAGACGTTCTGGACTGTGTTCTCCTTCTTATGTGGCTG | |
| | TGACTCCGTTTTCACTGCGTGGCGATAATGATGGCGGCG | |
| | GTGGCTCTTTTAGTACAGCAGATCAACTTGAAATGGTCA | |
| | CAGAACTCCTTGGTGGTGATATGGTTAATCAATCATTCA | |
| | TTTGTGATCCCGATGATGAGACATTTATAAAGAACATCA | |
| | TCATACAAGACTGCATGTGGTCAGGGTTTAGTGCTGCTG | |
| | CGAAACTGGTGAGCGAAAAGTTGGCTTCTTATCAAGCCG | |
| | CCCGGAAGGATAGTGGATCACCAAATCCAGCAAGGGGT | |
| | CATTCAGTGTGTAGCACAAGCTCTCTGTATCTTCAAGAC | |
| | CTCTCCGCGGCTGCAAGTGAATGTATTGATCCAAGTGTC | |
| | GTTTTCCCTTATCCCCTGAATGATTCTTCCTCTCCTAAAA | |
| | GCTGTGCGAGCCAGGATTCTTCAGCTTTCTCCCCAAGCT | |
| | CCGACAGTTTGTTGAGCTCTACTGAAAGTAGTCCTCAAG | |
| | GGTCACCGGAACCTCTCGTCCTTCACGAAGAAACACCCC | |
| | CTACAACTAGTTCCGATTCCGAAGAAGAACAGGAAGAC | |
| | GAAGAGGAGATTGACGTGGTATCAGTTGAGAAAAGACA | |
| | AGCCCCCGGGAAGCGAAGCGAAAGCGGGAGCCCAAGCG | |
| | CCGGCGGACATTCCAAGCCCCCACATTCTCCTTTGGTAC | |
| | TGAAAAGATGTCATGTGAGCACCCACCAACATAATTATG | |
| | CTGCTCCCCCATCAACCAGGAAAGATTACCCCGCCGCTA | |
| | AACGAGTTAAACTGGATTCAGTGAGGGTTCTTAGGCAAA | |
| | TTTCAAATAATAGGAAGTGTACTTCACCTCGCAGTAGCG | |
| | ATACAGAAGAAAACGTTAAAAGACGGACGCATAATGTG | |
| | CTGGAACGACAAAGACGAAATGAACTTAAGAGGTCCTT | |
| | CTTCGCGCTTAGGGATCAAATACCTGAACTGGAGAATAA | |
| | CGAGAAAGCTCCAAAAGTGGTGATTTTGAAGAAGGCTA | |
| | CTGCGTATATACTTTCTGTACAGGCCGAAGAACAGAAAC | |
| | TGATATCAGAGGAAGATCTGCTTCGTAAGCGCAGAGAG | |
| | CAACTGAAGCATAAGCTCGAGCAACTCCGCAATAGCTGC | |
| | GCCTAG | |
| KLF4 | ATGCGGCAGCCACCAGGGGAAAGTGATATGGCCGTTTCC | 4 |
| | GACGCTCTTCTGCCTTCATTTTCAACCTTTGCTTCCGGAC | |
| | CTGCCGGGCGGGAAAAGACGCTTAGGCAGGCCGGAGCA | |
| | CCCAACAATCGATGGAGAGAAGAACTGAGCCATATGAA | |
| | AAGACTGCCGCCTGTACTCCCGGGGCGGCCATACGATCT | |
| | CGCCGCCGCTACAGTAGCAACTGATTTGGAATCCGGTGG | |
| | TGCTGGGGCAGCCTGTGGCGGATCTAATCTTGCTCCTCT | |
| | GCCAAGACGGGAAACGGAAGAATTTAATGACCTGCTCG | |
| | ATTTGGATTTCATCCTTTCTAACTCACTTACACACCCACC | |
| | AGAAAGTGTCGCTGCTACGGTCTCTAGCTCTGCCAGCGC | |
| | AAGCAGTTCATCTTCTCCATCTTCCTCAGGACCCGCAAG | |
| | TGCCCCGAGCACTTGTTCCTTTACGTACCCAATACGAGC | |
| | TGGCAATGATCCTGGAGTTGCTCCCGGTGGTACAGGCGG | |
| | TGGTCTGTTGTACGGAAGAGAATCAGCCCCACCACCAAC | |
| | CGCACCATTTAATTTGGCCGATATTAATGATGTTTCACCT | |
| | AGTGGTGGTTTTGTTGCGGAACTGCTCCGTCCCGAGCTG | |
| | GATCCCGTCTATATCCCACCACAACAACCCCAACCACCT | |
| | GGCGGCGGATTGATGGGTAAATTTGTTCTTAAAGCATCC | |
| | CTTTCCGCACCAGGGTCAGAATATGGAAGTCCCTCAGTG | |
| | ATTTCCGTATCTAAGGGATCCCCCGATGGGTCCCATCCA | |
| | GTTGTCGTTGCCCCTTATAATGGTGGCCCGCCTAGAACC | |
| | TGTCCGAAAATAAAACAAGAAGCTGTATCATCATGTACG | |
| | CATCTCGGGGCAGGTCCACCCCTGTCTAACGGGCATCGC | |
| | CCTGCAGCCCATGATTTTCCTTTGGGCCGACAACTGCCG | |
| | TCCCGTACAACTCCAACACTCGGCCTCGAAGAGGTCCTC | |
| | AGTAGTAGAGATTGCCATCCAGCACTCCCCCTGCCACCA | |
| | GGTTTTCACCCTCATCCCGGTCCTAACTATCCCAGCTTTC | |
| | TCCCAGACCAAATGCAACCCCAGGTTCCCCCACTTCACT | |
| | ATCAGGGCCAAAGCAGAGGTTTCGTGGCGCGAGCCGGA | |
| | GAACCATGCGTCTGTTGGCCTCATTTTGGCACCCATGGA | |
| | ATGATGTTGACACCCCCAAGTAGTCCACTCGAACTGATG | |
| | CCCCCTGGCAGTTGTATGCCTGAAGAACCTAAACCCAAA | |
| | AGAGGGCGGCGGAGTTGGCCACGAAAGCGAACAGCAAC | |
| | TCATACCTGCGACTATGCTGGATGTGGGAAGACATATAC | |
| | CAAATCTAGCCACCTGAAAGCGCATCTCCGCACGCATAC | |
| | TGGAGAAAAGCCCTATCATTGCGATTGGGATGGGTGCGG | |
| | CTGGAAGTTTGCACGATCTGACGAGCTTACTAGACATTA | |
| | TCGCAAGCATACCGGACATCGGCCCTTTCAATGTCAGAA | |
| | GTGTGATCGCGCCTTCAGCCGGAGCGATCATCTGGCTCT | |
| | GCATATGAAACGACACTTCTAG | |
| LIN28 | ATGGGCTCAGTCAGCAATCAACAATTCGCGGGTGGATGT | 5 |
| | GCAAAAGCTGCTGAGGAAGCCCCAGAAGAAGCTCCAGA | |
| | AGATGCCGCTCGCGCCGCTGATGAACCACAACTGCTCCA | |
| | TGGAGCTGGGATTTGCAAATGGTTTAATGTCCGGATGGG | |
| | CTTTGGTTTCTTGTCTATGACAGCAAGAGCTGGAGTGGC | |
| | CCTCGATCCACCCGTCGACGTGTTCGTACATCAATCTAA | |
| | ACTTCATATGGAAGGATTTCGCTCACTCAAAGAAGGAGA | |
| | AGCCGTAGAATTTACATTCAAGAAAAGTGCGAAAGGGC | |
| | TTGAGAGCATACGGGTGACAGGGCCAGGCGGCGTGTTTT | |
| | GCATCGGATCAGAACGAAGGCCCAAGGGTAAATCCATG | |
| | CAAAAGAGGCGATCCAAAGGGGATCGGTGTTATAATTG | |
| | CGGTGGCCTTGACCACCACGCGAAAGAGTGTAAACTTCC | |
| | CCCGCAACCTAAGAAATGTCATTTCTGTCAATCCATATC | |
| | ACACATGGTGGCGAGCTGCCCATTGAAAGCACAACAAG | |
| | GACCGTCCGCCCAAGGGAAACCTACTTATTTCAGGGAAG | |
| | AGGAAGAGGAGATTCATAGTCCCACTCTCCTTCCTGAAG | |
| | CCCAAAACTAG | |
| NANOG | ATGTCAGTCGACCCGGCCTGCCCACAGAGTCTGCCCTGT | 6 |
| | TTCGAGGCTTCAGATTGCAAGGAGTCCTCTCCGATGCCC | |
| | GTCATCTGCGGACCCGAGGAGAATTACCCCAGTCTGCAG | |
| | ATGTCCAGTGCCGAAATGCCACATACAGAAACGGTTTCA | |
| | CCGCTCCCATCTTCAATGGACCTTTTGATCCAAGATTCTC | |
| | CCGACAGCAGCACTTCCCCAAAGGGAAAGCAGCCTACC | |
| | TCAGCGGAGAAATCTGTGGCCAAGAAAGAGGATAAAGT | |
| | TCCTGTTAAGAAGCAAAAGACAAGGACCGTATTTTCCTC | |
| | AACACAACTCTGCGTCCTTAACGACCGGTTCCAACGCCA | |
| | AAAGTATCTGTCCTTGCAACAAATGCAGGAGCTGAGCAA | |
| | TATTTTGAATCTGTCTTATAAGCAAGTCAAGACTTGGTTT | |
| | CAAAACCAGCGTATGAAGAGTAAAAGATGGCAGAAGAA | |
| | TAATTGGCCCAAGAACTCTAACGGCGTTACTCAAAAGGC | |
| | GAGTGCCCCCACTTATCCATCTCTGTATTCATCATATCAT | |
| | CAAGGCTGTTTGGTTAATCCCACGGGCAATCTGCCGATG | |
| | TGGTCCAATCAAACGTGGAATAACAGTACTTGGTCCAAT | |
| | CAAACTCAAAATATACAAAGTTGGTCAAATCATTCTTGG | |
| | AATACCCAAACGTGGTGTACTCAGAGCTGGAATAACCA | |
| | AGCATGGAACTCCCCATTTTACAATTGCGGCGAAGAGTC | |
| | ACTCCAAAGCTGTATGCAATTTCAACCCAACAGTCCCGC | |
| | ATCCGATCTTGAAGCAGCACTGGAAGCGGCCGGTGAGG | |
| | GGCTGAACGTGATTCAACAAACAACAAGATACTTCTCCA | |
| | CACCTCAGACTATGGACCTTTTCCTGAATTATTCAATGA | |
| | ATATGCAGCCCGAGGATGTCTAG | |
| Exemplary Tail | AAAAAAAAAAAAAAAAAAAAAAAAAAAAAAAAAAAAA | 7 |
| | AAAAAAAAAAAAAAAGCATAGCAGCTGACTAAAAAAA | |
| | AAAAAAAAAAAAAAAAAAAAAAAAAAAAAAAAAAAAA | |
| | AAAA | |
| Exemplary 5′ | TTGGACCCTCGTACAGAAGCTAATACGACTCACTATAGG | 8 |
| UTR | GAAATAAGAGAGAAAAGAAGAGTAAGAAGAAATATAA | |
| | GAGCCACCATG | |
| Exemplary 3′ | GCTGCCTTCTGCGGGGCTTGCCTTCTGGCCATGCCCTTCT | 9 |
| UTR | TCTCTCCCTTGCACCTGTACCTCTTGGTCTTTGAATAAAG | |
| | CCTGAGTAGGAAGTGAGGGTCTAGAACTAGTGTCGACG | |
| | C | |
| GLIS1 | ATGGCCGAAGCCAGAACATCTCTGTCTGCCCACTGTAGA | 10 |
| | GGCCCCCTGGCTACAGGTCTGCACCCCGACCTGGATCTG | |
| | CCTGGCCGGAGCTTGGCCACCCCTGCTCCTAGCTGCTAC | |
| | CTGCTGGGCTCTGAACCCAGCTCCGGCCTTGGCCTGCAG | |
| | CCTGAGACACACCTGCCTGAAGGCTCCCTCAAGCGGTGC | |
| | TGCGTGCTGGGCCTGCCTCCAACATCCCCTGCTTCCTCTT | |
| | CTCCATGCGCCAGCAGCGACGTGACCAGCATCATCAGAA | |
| | GCAGCCAGACAAGCCTCGTGACCTGCGTGAACGGCCTGC | |
| | GGAGCCCTCCCCTGACCGGCGATCTGGGAGGCCCCTCAA | |
| | AGAGAGCCCGGCCCGGCCCTGCCAGCACCGACAGCCAC | |
| | GAGGGCAGCCTGCAGCTGGAAGCTTGTAGAAAGGCCAG | |
| | CTTCCTGAAACAGGAGCCTGCCGACGAGTTCTCCGAGCT | |
| | GTTCGGCCCCCACCAGCAGGGACTGCCGCCTCCTTATCC | |
| | CCTGTCCCAGCTGCCCCCAGGACCAAGTCTGGGAGGACT | |
| | CGGCCTGGGACTGGCCGGCAGAGTAGTGGCCGGAAGAC | |
| | AGGCCTGCCGGTGGGTGGACTGTTGTGCCGCTTACGAGC | |
| | AGCAGGAGGAACTGGTGCGCCACATCGAGAAAAGCCAC | |
| | ATCGACCAGAGGAAGGGCGAGGATTTCACATGCTTCTGG | |
| | GCCGGCTGCGTGCGGCGGTACAAGCCCTTTAATGCCAGA | |
| | TACAAGCTGCTGATCCACATGAGAGTGCACAGCGGCGA | |
| | AAAGCCTAACAAGTGCATGTTCGAGGGCTGCAGCAAGG | |
| | CCTTCTCTAGACTGGAAAATCTGAAAATCCACCTGAGAT | |
| | CTCACACCGGAGAAAAGCCTTACCTGTGCCAGCATCCTG | |
| | GATGTCAGAAGGCCTTCAGCAACAGCTCTGATAGAGCCA | |
| | AGCACCAGAGAACCCACCTGGACACCAAGCCTTATGCCT | |
| | GCCAGATCCCTGGCTGCAGCAAAAGATACACCGATCCTT | |
| | CTAGCCTGCGGAAGCACGTGAAGGCCCATAGCGCTAAG | |
| | GAACAGCAGGTGCGCAAAAAGCTGCATGCCGGCCCAGA | |
| | TACCGAGGCCGATGTGCTAACTGAGTGCCTGGTCCTGCA | |
| | ACAGCTGCACACATCTACCCAGCTGGCCGCCAGCGACGG | |
| | CAAGGGCGGATGTGGCCTGGGACAGGAGCTGCTGCCTG | |
| | GTGTTTACCCCGGAAGCATCACCCCTCACAACGGCCTGG | |
| | CCAGCGGACTGCTGCCCCCCGCACACGACGTGCCTAGCC | |
| | GGCATCACCCTCTGGACGCCACCACCAGCAGCCACCACC | |
| | ACCTGAGCCCCCTGCCTATGGCCGAAAGCACCAGAGATG | |
| | GCCTCGGCCCTGGCCTCCTCAGCCCTATCGTGTCTCCACT | |
| | GAAAGGCCTGGGCCCCCCTCCTCTTCCTCCAAGCAGCCA | |
| | GAGCCACAGCCCCGGCGGCCAGCCTTTCCCTACCCTGCC | |
| | TTCTAAGCCCAGCTACCCTCCTTTTCAGAGCCCTCCTCCC | |
| | CCTCCACTGCCCTCTCCTCAGGGCTACCAAGGCAGCTTC | |
| | CACTCTATCCAGTCCTGTTTTCCTTACGGCGACTGCTACA | |
| | GAATGGCCGAACCAGCCGCCGGCGGCGACGGCCTGGTG | |
| | GGCGAGACACACGGCTTTAACCCCCTGAGACCTAACGGC | |
| | TACCACTCTCTGAGCACACCTCTGCCTGCTACCGGCTAC | |
| | GAGGCTCTGGCAGAGGCCAGCTGCCCTACCGCCCTGCCG | |
| | CAACAACCTAGCGAAGACGTCGTGTCCTCTGGCCCAGAG | |
| | GACTGCGGATTCTTCCCCAACGGCGCCTTCGACCACTGC | |
| | CTGGGCCACATTCCTAGCATCTACACCGACACATGA | |
| OCT4MyoD for | ATGGAGTTCGCCATGGAGCTGCTGTCGCCACCGTTGCGT | 11 |
| T-cells | GACGTGGACCTGACAGCCCCCGACGGCTCTCTGTGCTCC | |
| (T-OCT4MyoD) | TTTGCTACTACCGACGATTTCTACGATGACCCGTGCTTTG | |
| | ATTCTCCCGACCTGCGCTTTTTCGAAGACTTAGATCCGCG | |
| | CCTGATGCATGTAGGTGCTCTGCTAAAGCCCGAGGAGCA | |
| | CATGGCTGGCCACTTGGCTTCCGACTTCGCGTTCTCCCCG | |
| | CCACCCGGCGGCGGAGGCGATGGCCCTGGCGGACCCGA | |
| | GCCAGGTTGGGTCGACCCTCGGACCTGGCTCTCCTTCCA | |
| | GGGCCCCCCAGGCGGGCCCGGCATAGGTCCCGGCGTTG | |
| | GACCCGGGAGCGAGGTGTGGGGCATCCCTCCGTGCCCAC | |
| | CCCCGTATGAGTTCTGCGGGGGTATGGCCTACTGCGGGC | |
| | CTCAGGTGGGCGTCGGCCTCGTCCCTCAGGGGGGTCTGG | |
| | AGACCTCTCAGCCGGAGGGAGAGGCTGGGGTCGGCGTG | |
| | GAGAGCAACTCCGACGGGGCGTCGCCCGAACCTTGCACT | |
| | GTCACGCCCGGGGCCGTTAAGCTGGAGAAGGAGAAACT | |
| | TGAGCAGAACCCCGAGGAGAGCCAGGACATCAAGGCGC | |
| | TGCAGAAGGAGCTGGAACAGTTCGCCAAGCTGCTGAAG | |
| | CAGAAGCGCATCACCCTAGGTTACACCCAGGCGGACGT | |
| | GGGCCTGACGCTTGGTGTGCTGTTCGGAAAGGTGTTCAG | |
| | CCAGACGACCATCTGCCGATTCGAGGCCCTCCAGCTGTC | |
| | CTTCAAGAACATGTGCAAGTTGCGGCCCCTGCTCCAAAA | |
| | ATGGGTGGAGGAGGCTGACAACAACGAGAATCTCCAGG | |
| | AGATCTGTAAAGCCGAGACTCTGGTGCAGGCCCGCAAA | |
| | CGCAAGCGTACCTCGATTGAAAACAGGGTGCGTGGCAA | |
| | CCTGGAGAACCTGTTCCTACAGTGTCCCAAGCCTACCCT | |
| | TCAGCAGATTAGCCACATCGCACAACAGTTGGGCCTTGA | |
| | AAAGGATGTGGTACGCGTGTGGTTCTGTAACCGCCGCCA | |
| | GAAGGGTAAGCGCAGCTCCAGCGACTACGCGCAGAGAG | |
| | AGGACTTTGAGGCTGCAGGATCTCCTTTTTCTGGCGGCC | |
| | CTGTGAGTTTCCCTCTGGCCCCGGGACCCCACTTTGGTAC | |
| | TCCGGGCTACGGCTCCCCGCACTTCACCGCCCTGTACTC | |
| | TAGTGTCCCGTTTCCCGAGGGCGAGGCGTTCCCCCCAGT | |
| | GTCCGTGACCACACTGGGGTCCCCAATGCATTCAAATTG | |
| | A | |
| B18R for T cells | ATGACAATGAAAATGATGGTGCACATCTACTTCGTCTCT | 12 |
| (T-B18R) | CTTCTGCTGCTGCTGTTTCACAGCTACGCAATTGACATCG | |
| | AAAACGAGATCACCGAGTTCTTCAACAAGATGCGTGAC | |
| | ACCCTTCCCGCCAAGGATTCTAAATGGCTCAACCCGGCC | |
| | TGCATGTTTGGAGGCACCATGAACGACATCGCGGCGCTG | |
| | GGCGAGCCCTTCTCCGCTAAATGTCCCCCGATCGAAGAT | |
| | TCTCTGCTGAGCCACCGCTACAAGGACTATGTGGTCAAA | |
| | TGGGAGCGCCTGGAGAAGAACCGTCGGCGCCAGGTCTC | |
| | AAACAAGCGCGTAAAGCATGGAGATCTTTGGATCGCCA | |
| | ACTACACCAGTAAATTTAGCAACCGCCGCTACCTGTGCA | |
| | CTGTCACCACCAAGAATGGGGACTGCGTGCAGGGCATC | |
| | GTGCGGAGCCACATCCGCAAGCCTCCCTCTTGTATTCCC | |
| | AAGACCTACGAGCTGGGGACACATGACAAGTACGGCAT | |
| | TGACCTGTATTGCGGGATCCTGTACGCGAAGCACTACAA | |
| | CAACATCACCTGGTACAAGGACAACAAGGAGATTAACA | |
| | TCGATGACATCAAGTACTCCCAGACTGGCAAGGAGCTCA | |
| | TCATCCACAACCCTGAGCTGGAGGACTCCGGTAGATATG | |
| | ATTGTTACGTGCATTACGACGATGTGCGCATCAAAAACG | |
| | ACATTGTCGTTTCCAGGTGTAAGATACTCACTGTGATCC | |
| | CTAGTCAGGACCACCGCTTCAAGCTGATCTTGGACCCGA | |
| | AGATAAATGTGACCATCGGGGAGCCAGCCAATATCACCT | |
| | GCACTGCCGTGTCCACCTCGTTGCTCATTGACGATGTACT | |
| | GATTGAGTGGGAGAATCCATCGGGTTGGCTAATCGGCTT | |
| | CGACTTCGACGTGTACTCGGTGCTAACCTCCCGTGGTGG | |
| | CATCACTGAGGCTACGCTTTATTTCGAGAACGTGACGGA | |
| | GGAGTACATCGGCAACACCTACAAGTGCCGAGGCCACA | |
| | ACTACTACTTCGAAAAAACCCTGACGACGACCGTGGTTT | |
| | TGGAGTGA | |
| KLF4 for T cells | ATGCGTCAGCCACCCGGGGAGAGCGACATGGCCGTGTC | 13 |
| (T-KLF4) | GGACGCGCTGCTGCCATCCTTTTCCACCTTCGCCTCGGGT | |
| | CCGGCCGGCCGAGAGAAGACTCTGCGCCAGGCCGGAGC | |
| | CCCTAACAACCGCTGGAGAGAGGAGCTGTCACACATGA | |
| | AACGCCTGCCCCCCGTGCTGCCTGGGCGCCCCTACGACC | |
| | TTGCCGCGGCCACGGTGGCTACCGACTTGGAGTCTGGAG | |
| | GTGCTGGAGCAGCGTGTGGCGGAAGCAACCTGGCACCG | |
| | TTGCCACGCCGGGAGACCGAGGAGTTCAACGACTTGTTG | |
| | GATCTGGACTTTATTCTGTCCAACTCCCTTACACACCCGC | |
| | CCGAGAGCGTAGCAGCCACCGTGAGCTCCAGTGCTTCCG | |
| | CTTCCTCCTCATCCAGCCCGTCGTCTTCTGGCCCTGCCTC | |
| | TGCGCCGTCGACCTGTTCGTTCACCTATCCCATCCGGGCC | |
| | GGCAACGATCCGGGCGTGGCCCCGGGCGGCACCGGTGG | |
| | TGGTCTCCTGTACGGCAGGGAGTCCGCCCCCCCTCCAAC | |
| | CGCTCCCTTCAACCTCGCGGACATCAATGACGTGTCCCC | |
| | CTCTGGCGGCTTCGTTGCAGAACTGTTAAGGCCTGAACT | |
| | GGATCCAGTGTACATCCCGCCCCAGCAGCCTCAGCCGCC | |
| | GGGCGGCGGTCTGATGGGCAAATTTGTCCTGAAGGCGTC | |
| | TCTGTCTGCTCCTGGCTCCGAGTACGGCAGCCCCAGTGT | |
| | GATTAGCGTGTCTAAAGGCAGCCCCGACGGGTCGCACCC | |
| | CGTGGTGGTCGCTCCTTACAACGGTGGACCCCCGCGCAC | |
| | CTGCCCAAAGATCAAGCAGGAGGCTGTTTCTTCATGCAC | |
| | TCATCTAGGCGCCGGTCCACCCCTTTCCAATGGCCACCG | |
| | GCCCGCCGCGCATGACTTCCCCCTGGGCCGCCAGCTGCC | |
| | CAGCCGGACCACACCTACCTTAGGCCTGGAGGAGGTGCT | |
| | TAGTTCGCGCGACTGTCATCCTGCCCTGCCTCTCCCTCCA | |
| | GGCTTCCACCCCCACCCGGGACCCAACTACCCGTCCTTC | |
| | CTGCCCGACCAGATGCAACCTCAGGTCCCTCCCCTGCAC | |
| | TACCAGGGACAGAGCCGCGGTTTCGTCGCCCGTGCTGGC | |
| | GAGCCATGCGTCTGTTGGCCGCACTTCGGCACGCATGGA | |
| | ATGATGCTAACTCCCCCGAGCTCCCCCCTGGAGCTGATG | |
| | CCCCCGGGTTCCTGTATGCCAGAGGAGCCCAAGCCTAAG | |
| | CGCGGCAGACGTAGTTGGCCCCGTAAGCGCACCGCCAC | |
| | ACATACGTGCGACTACGCGGGGTGCGGGAAAACCTACA | |
| | CCAAGTCTTCTCACCTCAAGGCTCACTTGCGTACTCACA | |
| | CTGGAGAGAAGCCTTATCACTGCGACTGGGATGGGTGCG | |
| | GGTGGAAGTTTGCTCGCTCCGACGAACTCACCCGCCATT | |
| | ACCGCAAGCACACCGGCCACCGCCCCTTCCAGTGTCAGA | |
| | AGTGCGATCGAGCGTTCTCCCGCTCGGACCACCTGGCCC | |
| | TACACATGAAGAGGCACTTTTGA | |
| LIN28 for T cells | ATGGGCTCCGTGTCGAATCAGCAGTTCGCAGGCGGGTGC | 14 |
| (T-LIN28) | GCCAAGGCAGCCGAGGAGGCCCCGGAGGAGGCTCCTGA | |
| | AGACGCCGCTCGCGCGGCGGACGAGCCTCAGCTACTCC | |
| | ATGGAGCTGGCATCTGCAAATGGTTCAACGTGCGAATGG | |
| | GATTTGGCTTCCTGTCTATGACTGCTCGTGCCGGCGTGGC | |
| | GCTGGACCCGCCCGTAGACGTGTTCGTCCACCAGAGCAA | |
| | GCTGCATATGGAGGGTTTCAGATCTCTGAAGGAGGGCGA | |
| | GGCCGTGGAGTTCACGTTCAAGAAGTCGGCCAAAGGTCT | |
| | GGAGAGCATCCGCGTCACCGGTCCCGGCGGCGTTTTTTG | |
| | TATTGGCTCCGAGCGCCGGCCCAAGGGCAAGTCCATGCA | |
| | GAAGCGCCGCTCCAAGGGGGACAGGTGTTACAACTGCG | |
| | GGGGTTTGGATCACCACGCGAAGGAGTGCAAACTTCCTC | |
| | CTCAGCCGAAGAAATGTCACTTTTGTCAGAGCATCAGTC | |
| | ACATGGTGGCTTCATGCCCACTCAAGGCCCAACAGGGAC | |
| | CCTCTGCGCAGGGCAAGCCCACCTACTTCCGCGAGGAGG | |
| | AAGAAGAGATCCACTCCCCAACCCTGCTGCCCGAGGCTC | |
| | AGAACTGA | |
| NANOG for T | ATGTCTGTTGACCCGGCCTGCCCACAGAGCCTTCCCTGC | 15 |
| cells | TTTGAGGCTTCCGACTGTAAAGAGAGTTCCCCGATGCCC | |
| (T-NANOG) | GTGATTTGTGGTCCTGAAGAGAACTACCCGTCCCTACAG | |
| | ATGTCATCGGCGGAGATGCCTCATACCGAGACCGTGTCC | |
| | CCCTTACCCTCTTCTATGGATCTGCTGATCCAGGACTCGC | |
| | CCGACAGCTCGACCAGCCCTAAGGGCAAGCAGCCAACC | |
| | TCCGCAGAGAAGTCCGTGGCTAAGAAGGAGGACAAAGT | |
| | ACCGGTGAAGAAACAGAAGACTCGCACCGTGTTCTCGTC | |
| | GACCCAGCTGTGCGTGCTCAACGACAGGTTTCAGCGCCA | |
| | GAAGTACCTGTCCCTGCAGCAGATGCAAGAGCTCAGCA | |
| | ACATCCTGAACCTGAGCTACAAGCAGGTCAAGACCTGGT | |
| | TCCAGAACCAGCGTATGAAGTCCAAGCGGTGGCAAAAA | |
| | AACAACTGGCCCAAGAACAGCAACGGTGTCACCCAGAA | |
| | GGCCTCCGCCCCCACCTACCCGTCTCTTTACTCCAGCTAT | |
| | CACCAGGGCTGCCTGGTGAATCCAACAGGAAACCTGCCC | |
| | ATGTGGTCTAATCAGACCTGGAACAACTCCACCTGGTCT | |
| | AACCAGACACAGAACATCCAGAGTTGGTCTAATCACTCT | |
| | TGGAACACGCAGACCTGGTGCACTCAGAGTTGGAATAA | |
| | CCAGGCCTGGAATTCCCCCTTCTACAACTGCGGGGAGGA | |
| | GAGCTTGCAGTCATGTATGCAGTTCCAGCCCAACTCCCC | |
| | AGCCAGTGACCTGGAGGCCGCGCTGGAGGCGGCTGGCG | |
| | AGGGCCTCAACGTGATCCAACAGACGACCCGCTACTTCT | |
| | CCACGCCTCAGACTATGGATTTGTTCCTGAACTATTCGAT | |
| | GAACATGCAGCCTGAAGATGTGTGA | |
| OCT4 for T cells | ATGGCGGGCCACTTGGCTTCCGACTTCGCGTTCTCTCCCC | 16 |
| (T-OCT4) | CGCCTGGCGGAGGCGGGGACGGCCCCGGCGGCCCTGAG | |
| | CCAGGCTGGGTCGATCCACGCACCTGGCTCTCCTTCCAG | |
| | GGACCCCCCGGGGGGCCCGGCATCGGTCCCGGGGTAGG | |
| | CCCCGGATCGGAGGTGTGGGGCATCCCGCCGTGCCCACC | |
| | GCCCTATGAGTTCTGTGGGGGTATGGCCTACTGCGGGCC | |
| | TCAGGTCGGTGTCGGTCTGGTGCCTCAGGGCGGTCTCGA | |
| | GACCAGCCAGCCGGAGGGCGAGGCTGGCGTGGGTGTGG | |
| | AGAGCAACAGCGATGGCGCTTCTCCTGAACCATGCACTG | |
| | TCACCCCCGGCGCCGTGAAGCTGGAGAAAGAGAAACTG | |
| | GAGCAGAATCCAGAGGAGAGTCAGGACATCAAGGCCCT | |
| | GCAGAAGGAACTGGAACAGTTCGCCAAGCTGCTGAAGC | |
| | AGAAGCGTATCACCCTTGGATACACCCAGGCGGACGTG | |
| | GGCCTCACTTTAGGCGTTCTTTTCGGAAAGGTGTTCTCAC | |
| | AGACCACAATCTGCCGATTCGAGGCCCTGCAACTTTCTT | |
| | TCAAAAACATGTGCAAGTTGCGGCCCCTACTGCAGAAAT | |
| | GGGTGGAGGAAGCGGACAACAACGAGAATCTGCAGGAG | |
| | ATCTGTAAGGCCGAGACACTGGTGCAGGCTCGCAAGCG | |
| | CAAAAGAACGAGCATTGAGAACCGCGTCCGCGGCAACC | |
| | TGGAGAACCTGTTCCTGCAGTGCCCAAAGCCTACCCTCC | |
| | AGCAAATTAGCCACATCGCTCAGCAGCTGGGCCTGGAG | |
| | AAGGATGTGGTGAGGGTGTGGTTCTGTAACCGGCGCCAG | |
| | AAGGGCAAGCGCAGCTCCAGTGACTACGCACAGCGTGA | |
| | GGACTTTGAAGCCGCCGGCTCCCCGTTTTCAGGAGGCCC | |
| | CGTGTCCTTTCCTTTGGCTCCCGGCCCTCATTTCGGTACT | |
| | CCGGGCTACGGCTCCCCCCACTTTACCGCCCTTTACTCCT | |
| | CTGTCCCCTTCCCCGAGGGAGAGGCATTTCCCCCGGTGT | |
| | CCGTAACGACCCTGGGTTCCCCAATGCACTCTAACTGA | |
| SOX2 for T cells | ATGTATAACATGATGGAAACAGAGCTGAAGCCCCCGGG | 17 |
| (T-SOX2) | GCCTCAACAGACCTCCGGCGGTGGGGGCGGCAACTCGA | |
| | CCGCTGCCGCAGCTGGTGGAAACCAGAAGAACAGTCCC | |
| | GACAGAGTTAAGCGCCCGATGAACGCGTTCATGGTGTGG | |
| | TCTCGCGGCCAGCGCCGCAAGATGGCGCAGGAGAATCC | |
| | AAAAATGCACAACTCGGAGATCTCCAAGCGGCTCGGTG | |
| | CCGAGTGGAAGCTGCTAAGCGAGACCGAGAAACGTCCT | |
| | TTTATTGACGAGGCCAAGCGCCTGCGTGCGCTTCACATG | |
| | AAGGAGCACCCCGACTACAAGTACAGGCCCCGACGCAA | |
| | AACCAAGACCCTGATGAAAAAGGACAAGTACACCCTCC | |
| | CCGGCGGCCTGCTGGCCCCTGGTGGCAACAGCATGGCCT | |
| | CCGGAGTCGGGGTAGGCGCCGGCCTTGGAGCTGGAGTC | |
| | AACCAGCGTATGGATTCTTACGCGCACATGAATGGGTGG | |
| | TCAAATGGCTCGTATTCTATGATGCAGGACCAGCTGGGC | |
| | TACCCTCAACACCCCGGCCTCAACGCCCATGGAGCGGCT | |
| | CAGATGCAGCCAATGCACCGCTACGATGTGAGCGCCCTG | |
| | CAGTACAACTCTATGACTAGTTCACAGACTTACATGAAC | |
| | GGTTCCCCAACCTACTCCATGTCTTACAGTCAGCAGGGA | |
| | ACGCCGGGTATGGCTCTGGGCTCCATGGGCTCCGTGGTG | |
| | AAGTCGGAGGCATCCTCCAGCCCTCCCGTGGTCACCTCC | |
| | TCCTCTCACAGCCGCGCTCCTTGCCAGGCCGGGGACCTG | |
| | CGCGACATGATCTCTATGTATCTGCCCGGTGCAGAGGTG | |
| | CCTGAACCGGCGGCCCCCTCTCGGTTGCATATGTCCCAG | |
| | CATTACCAGAGCGGCCCGGTGCCAGGCACTGCCATCAAC | |
| | GGCACCTTGCCCCTGAGCCACATGTGA | |
| cMYC for T-cells | ATGGATTTCTTCCGAGTGGTGGAGAATCAGCAGCCGCCT | 18 |
| (T-cMyc) | GCCACCATGCCCCTTAACGTGTCCTTCACTAACAGAAAC | |
| | TACGACCTGGACTACGACAGTGTCCAGCCCTATTTCTAC | |
| | TGTGATGAGGAGGAGAACTTTTACCAGCAGCAACAGCA | |
| | GAGCGAACTGCAGCCCCCAGCGCCCTCGGAGGACATCT | |
| | GGAAGAAATTTGAGCTGCTGCCAACCCCCCCCCTATCTC | |
| | CATCCCGCCGTTCCGGTCTCTGCTCTCCTAGCTACGTGGC | |
| | TGTCACTCCCTTTTCGCTGCGTGGGGACAACGATGGGGG | |
| | GGGCGGAAGTTTCAGCACTGCGGACCAGCTGGAGATGG | |
| | TGACCGAGCTGCTGGGTGGTGACATGGTCAACCAGTCTT | |
| | TTATCTGCGACCCGGATGACGAGACCTTCATCAAGAACA | |
| | TCATCATCCAGGACTGTATGTGGTCTGGCTTCTCTGCCGC | |
| | TGCAAAACTGGTCTCGGAGAAACTTGCTAGCTACCAGGC | |
| | TGCTCGCAAGGACTCCGGCTCGCCGAATCCAGCTAGGGG | |
| | ACATAGTGTTTGTAGTACCTCCTCGCTCTACCTGCAGGA | |
| | CCTGTCCGCAGCGGCCTCTGAGTGTATTGACCCGTCCGT | |
| | GGTGTTCCCCTATCCTCTCAACGACTCGTCTTCCCCCAAG | |
| | AGCTGCGCCTCCCAGGACTCATCTGCGTTCTCCCCCTCCT | |
| | CCGATAGCCTGTTGAGTAGCACAGAGAGCTCCCCTCAGG | |
| | GCTCCCCGGAGCCCCTGGTGCTACACGAGGAGACCCCGC | |
| | CCACCACCAGCAGTGACTCAGAAGAGGAGCAGGAGGAC | |
| | GAAGAGGAGATCGATGTGGTTTCCGTGGAGAAGCGCCA | |
| | GGCCCCTGGCAAACGCTCCGAATCCGGCTCCCCTTCCGC | |
| | CGGCGGCCACTCCAAGCCCCCGCACAGCCCGTTGGTGCT | |
| | GAAGAGATGTCACGTGTCAACCCACCAGCATAACTACGC | |
| | GGCGCCTCCCTCTACCCGCAAAGACTACCCCGCTGCCAA | |
| | GCGCGTGAAGTTGGATAGCGTCCGGGTCTTACGCCAGAT | |
| | TTCTAACAACCGCAAGTGCACTTCTCCAAGGTCTTCCGA | |
| | CACGGAGGAGAACGTGAAGCGGCGCACCCACAACGTAC | |
| | TGGAGCGCCAGCGTCGTAACGAGTTGAAGCGCAGCTTCT | |
| | TCGCGCTTCGGGACCAGATACCTGAGCTCGAGAATAACG | |
| | AGAAAGCACCAAAGGTAGTCATCCTGAAGAAGGCCACG | |
| | GCCTACATCTTATCAGTGCAGGCCGAGGAGCAGAAGCTG | |
| | ATTTCAGAGGAGGATCTGCTGCGCAAGCGCCGAGAACA | |
| | GCTGAAGCACAAGCTGGAACAACTCCGCAACTCATGCG | |
| | CCTGA | |
| GLIS1 for T cells | ATGGCCGAGGCCCGCACTAGCCTCTCAGCGCACTGTCGC | 19 |
| (T-GLIS1) | GGCCCTCTCGCTACGGGCCTGCACCCCGACCTGGACCTG | |
| | CCCGGGCGCAGCCTGGCTACCCCGGCCCCCTCCTGTTAC | |
| | CTTTTGGGCTCGGAGCCCTCTAGCGGCCTGGGCCTACAG | |
| | CCAGAGACTCACCTTCCCGAAGGCTCCCTGAAGAGGTGT | |
| | TGCGTCCTTGGCTTGCCGCCCACCTCGCCTGCCAGCTCCT | |
| | CCCCATGCGCGTCTTCAGACGTGACCTCCATCATCCGCT | |
| | CGTCCCAGACCTCCCTGGTGACCTGCGTGAACGGGCTGC | |
| | GTTCTCCCCCGCTCACCGGCGACCTGGGAGGTCCGAGTA | |
| | AGCGCGCTCGGCCCGGCCCTGCGAGTACAGACTCTCATG | |
| | AGGGCTCGCTGCAGCTGGAAGCGTGTCGCAAGGCCTCCT | |
| | TCCTGAAGCAGGAGCCAGCCGACGAGTTCTCTGAGCTGT | |
| | TCGGACCTCACCAGCAGGGTTTGCCCCCTCCTTACCCAC | |
| | TCAGCCAGCTCCCCCCTGGGCCCTCTTTGGGCGGTTTAG | |
| | GCCTGGGGTTAGCGGGACGTGTGGTGGCTGGCCGCCAG | |
| | GCCTGCCGATGGGTCGATTGTTGTGCCGCTTACGAGCAG | |
| | CAGGAGGAGCTGGTGCGCCACATTGAAAAGAGCCATAT | |
| | TGACCAGCGCAAGGGCGAGGACTTCACCTGCTTTTGGGC | |
| | TGGGTGCGTGCGCCGCTACAAGCCGTTCAACGCGAGATA | |
| | CAAGCTGCTGATCCACATGCGTGTACACTCCGGAGAGAA | |
| | ACCCAACAAGTGCATGTTTGAGGGATGCTCCAAGGCGTT | |
| | CAGCCGCCTGGAGAACTTGAAAATCCACCTGCGCTCACA | |
| | CACCGGTGAGAAGCCATACCTGTGCCAACACCCCGGTTG | |
| | TCAGAAGGCGTTCAGCAACAGCTCCGATCGAGCTAAAC | |
| | ACCAGCGGACCCATCTTGACACCAAGCCGTACGCATGCC | |
| | AAATTCCAGGGTGCTCCAAGCGTTACACCGACCCGTCCA | |
| | GTCTGCGCAAGCACGTGAAGGCCCATAGTGCGAAGGAG | |
| | CAGCAAGTGCGGAAGAAGCTCCACGCCGGCCCTGACAC | |
| | GGAGGCCGATGTGCTGACCGAGTGCCTGGTTCTGCAGCA | |
| | GCTGCACACTAGCACCCAGCTGGCAGCCAGCGATGGCA | |
| | AAGGTGGTTGCGGGCTGGGACAGGAGTTACTGCCAGGC | |
| | GTGTACCCCGGAAGCATCACCCCGCACAACGGCCTAGCC | |
| | TCTGGACTTCTCCCTCCTGCCCATGACGTGCCCTCTCGGC | |
| | ACCACCCGCTCGATGCTACCACCTCCTCTCACCACCACC | |
| | TGTCCCCCTTGCCTATGGCCGAGTCCACCCGCGACGGCC | |
| | TGGGCCCTGGCTTGTTGTCGCCCATCGTGTCCCCTCTGAA | |
| | AGGCCTTGGCCCCCCGCCGCTGCCCCCCTCTTCTCAGTCC | |
| | CATAGTCCCGGTGGGCAGCCGTTTCCCACCCTGCCCTCT | |
| | AAGCCCTCCTATCCCCCGTTCCAGAGTCCACCCCCCCCC | |
| | CCACTGCCGTCCCCGCAGGGCTACCAGGGCTCCTTCCAT | |
| | TCTATCCAGTCTTGCTTTCCTTACGGCGATTGTTACAGGA | |
| | TGGCTGAGCCCGCAGCTGGGGGCGATGGGCTGGTGGGC | |
| | GAGACACATGGATTCAATCCACTCAGGCCCAACGGTTAT | |
| | CACTCGCTATCCACTCCGTTGCCTGCCACGGGTTATGAG | |
| | GCGCTGGCAGAGGCTAGCTGCCCAACGGCTCTGCCTCAG | |
| | CAGCCATCAGAGGACGTGGTCAGCAGCGGCCCTGAAGA | |
| | CTGCGGCTTCTTTCCTAATGGGGCCTTCGACCACTGTCTT | |
| | GGCCACATCCCGTCCATCTACACAGACACCTGA | |

### DETAILED DESCRIPTION

### I. Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The term “about”, particularly in reference to a given quantity, is meant to encompass deviations of plus or minus five percent.

The compositions of the present disclosure can comprise, consist essentially of, or consist of, the components disclosed.

All percentages, parts and ratios are based upon the total weight of the topical compositions and all measurements made are at about 25° C., unless otherwise specified.

As used herein, the term “cell” refers to an intact live cell, naturally occurring or modified. The cell may be isolated from other cells, mixed with other cells in a culture, or within a tissue (partial or intact), or an organism. The methods described herein can be performed, for example, on a sample comprising a single cell, a population of cells, or a tissue or organ comprising cells.

As used herein, the term “mammalian cell” refers to any cell derived from a mammalian subject suitable for transplantation into the same or a different subject. The cell may be xenogeneic, autologous, or allogeneic. The cell can be a primary cell obtained directly from a mammalian subject. The cell may also be a cell derived from the culture and expansion of a cell obtained from a subject. In some embodiments, the cell has been genetically engineered to express a recombinant protein and/or nucleic acid.

As used herein, the term “non-integrative” with reference to a messenger RNA (mRNA) refers to an mRNA molecule that is not integrated intrachromosomally nor extrachromosomally into the host genome.

The phrase “pharmaceutically acceptable” is employed herein to refer to those compounds, salts, compositions, dosage forms, etc., which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in mammals (e.g., animals), and more particularly, in humans.

As used herein, the term “rejuvenated cell(s)” refers to aged cells that have been treated or transiently reprogrammed with one or more cellular reprogramming factors such that the cells have a transcriptomic profile of a younger cell while still retaining one or more cell identity markers.

As used herein, the term “replication element” is a sequence and/or motif(s) necessary or useful for replication of and/or that initiates transcription of a synthetic, RNA vector, such as a self-replicating RNA or a circular polyribonucleotide.

The term “somatic cell” refers to any cell other than a germ cell, a cell present in or obtained from a pre-implantation embryo, or a cell resulting from proliferation of such a cell in vitro. Stated another way, a somatic cell refers to any cell forming the body of an organism, except for a germline cell. In mammals, germline cells (also known as “gametes”) are the spermatozoa and ova. Every other cell type in the mammalian body—apart from the sperm and ova, the cells from which they are made (gametocytes) and undifferentiated, pluripotent, embryonic stem cells—is a somatic cell: internal organs, skin, bones, blood, and connective tissue are all made up of somatic cells. In some embodiments the somatic cell is an “adult somatic cell,” by which is meant a cell that is present in or obtained from an organism other than an embryo or a fetus or results from proliferation of such a cell in vitro. Unless otherwise indicated, the compositions and methods for rejuvenating a somatic cell can be performed both in vivo and in vitro, where in vivo is practiced when a somatic cell is present within a subject, and where in vitro is practiced using an isolated somatic cell maintained in culture.

As used herein, the term “stagger element” is a moiety, such as a nucleotide sequence, that induces ribosomal pausing during translation. In some embodiments, the stagger element is a non-conserved sequence of amino-acids with a strong alpha-helical propensity followed by the consensus sequence −D(V/I)ExNPG P, where x=any amino acid. In some embodiments, the stagger element may include a chemical moiety, such as glycerol, a non nucleic acid linking moiety, a chemical modification, a modified nucleic acid, or any combination thereof.

As used herein, the term “stem cell” refers to a cell that retains the ability to renew itself through mitotic cell division and that can differentiate into a diverse range of specialized cell types. Mammalian stem cells can be divided into three broad categories: embryonic stem cells, which are derived from blastocysts, adult stem cells, which are found in adult tissues, and cord blood stem cells, which are found in the umbilical cord. In a developing embryo, stem cells can differentiate into all of the specialized embryonic tissues. In adult organisms, stem cells and progenitor cells act as a repair system for the body by replenishing specialized cells. Totipotent stem cells are produced from the fusion of an egg and sperm cell. Cells produced by the first few divisions of the fertilized egg are also totipotent. These cells can differentiate into embryonic and extraembryonic cell types. Pluripotent stem cells are the descendants of totipotent cells and can differentiate into cells derived from any of the three germ layers. Multipotent stem cells can produce only cells of a closely related family of cells (e.g., hematopoietic stem cells differentiate into red blood cells, white blood cells, platelets, etc.). Unipotent cells can produce only one cell type, but have the property of self-renewal, which distinguishes them from non-stem cells. Induced pluripotent stem cells are a type of pluripotent stem cell derived from adult cells that have been reprogrammed into an embryonic-like pluripotent state. Induced pluripotent stem cells can be derived, for example, from adult somatic cells such as skin or blood cells.

As used herein, the term “termination element” is a moiety, such as a nucleic acid sequence, that terminates translation of the expression sequence in an RNA vector, such as a self-replicating RNA or a circular polyribonucleotide.

As used herein, the term “transfection” refers to the uptake of exogenous DNA or RNA by a cell. A cell has been “transfected” when exogenous DNA or RNA has been introduced inside the cell membrane. A number of transfection techniques are generally known in the art. See, e.g., Graham et al. (1973) Virology, 52:456, Sambrook et al. (2001) Molecular Cloning, a laboratory manual, 3.sup.rd edition, Cold Spring Harbor Laboratories, New York, Davis et al. (1995) Basic Methods in Molecular Biology, 2.sup.nd edition, McGraw-Hill, and Chu et al. (1981) Gene 13:197. Such techniques can be used to introduce one or more exogenous DNA or RNA molecules into cells. The term refers to both stable and transient uptake of the DNA or RNA molecules. For example, transfection can be used for transient uptake of mRNAs encoding cellular reprogramming factors into cells in need of rejuvenation.

As used herein, the term “translation initiation sequence” is a nucleic acid sequence that initiates translation of an expression sequence in an RNA vector, such as a self-replicating RNA or a circular polyribonucleotide.

As used herein, the term “transient reprogramming” refers to exposure of cells to cellular reprogramming factors for a period of time sufficient to rejuvenate cells (i.e., eliminate all or some hallmarks of aging), but not long enough to cause dedifferentiation into stem cells. Such transient reprogramming results in rejuvenated cells that retain their identity (i.e., differentiated cell-type).

As used herein, the term “cellular reprogramming factors” refers to a set of transcription factors, and combinations thereof, that can convert adult or differentiated cells into pluripotent stem cells. Exemplary reprogramming factors include OCT4, SOX2, KLF4, c-MYC, LIN28, NANOG and/or GLIS1. Other exemplary reprogramming factors include CMYC, DPPA2, DPPA4, ESRRB, GDF3, GLIS1, KLF2, KLF4, KLF5, LIN28, LMYC, NANOG, NMYC, NR5A1, NR5A2, OCT-4, RCOR2, SALL1, SALL4, SOX1, SOX2, SOX3, TDRD12, TET1, TH2A, TH2B, UTF1, ZFP42, MDM2, CyclinD1, SV40 large T antigen, SIRT6, TCL1A, and RARy.

The term “treating” is used herein, for instance, in reference to methods of treating a cell, a tissue or a subject, and generally includes the administration of a compound or composition which reduces the frequency of, or delays the onset of, symptoms of aging or of a medical condition in a subject relative to a subject not receiving the compound or composition. This can include reversing, reducing, or arresting the symptoms, clinical signs, and underlying pathology of a condition in a manner to improve or stabilize a subject's condition.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

### II. Methods of Treatment

In embodiments, the methods provided herein may be applied to any type of cell in need of rejuvenation. The cell may be isolated from other cells, mixed with other cells in a culture, or within a tissue (partial or intact), or a live organism. The methods described herein can be performed, for example, on a sample comprising a single cell, a population of cells, or a tissue or organ comprising cells. The cells chosen for rejuvenation will depend on the desired therapeutic effect for treating an age-related disease or condition.

In embodiments, the cells are mammalian cells. In embodiments, the cells are human cells. In embodiments, the cells are from an elderly subject.

In embodiments, the methods provided herein may be performed on cells, tissue, or organs of the nervous system, muscular system, respiratory system, cardiovascular system, skeletal system, reproductive system, integumentary system, lymphatic system, excretory system, endocrine system (e.g. endocrine and exocrine), or digestive system. Any type of cell can potentially be rejuvenated, as described herein, including, but not limited to, epithelial cells (e.g., squamous, cuboidal, columnar, and pseudostratified epithelial cells), endothelial cells (e.g., vein, artery, and lymphatic vessel endothelial cells), and cells of connective tissue, muscles, and the nervous system. Such cells may include, but are not limited to, epidermal cells, fibroblasts, chondrocytes, skeletal muscle cells, satellite cells, heart muscle cells, smooth muscle cells, keratinocytes, basal cells, ameloblasts, exocrine secretory cells, myoepithelial cells, osteoblasts, osteoclasts, neurons (e.g., sensory neurons, motor neurons, and interneurons), glial cells (e.g., oligodendrocytes, astrocytes, ependymal cells, microglia, Schwann cells, and satellite cells), pillar cells, adipocytes, pericytes, stellate cells, pneumocytes, blood and immune system cells (e.g., erythrocytes, monocytes, dendritic cells, macrophages, neutrophils, eosinophils, mast cells, T cells, B cells, natural killer cells), hormone-secreting cells, germ cells, interstitial cells, lens cells, photoreceptor cells, taste receptor cells, and olfactory cells; as well as cells and/or tissue from the kidney, liver, pancreas, stomach, spleen, gall bladder, intestines, bladder, lungs, prostate, breasts, urogenital tract, pituitary cells, oral cavity, esophagus, skin, hair, nail, thyroid, parathyroid, adrenal gland, eyes, nose, or brain.

In some embodiments, the cells are selected from fibroblasts, endothelial cells, chondrocytes, skeletal muscle stem cells, keratinocytes, mesenchymal stem cells and corneal epithelial cells. In embodiments, the cells are fibroblasts. In embodiments, the cells are endothelial cells. In embodiments, the cells are chondrocytes. In embodiments, the cells are skeletal muscle stem cells. In embodiments, the cells are keratinocytes. In embodiments, the cells are mesenchymal stem cells. In embodiments, the cells are corneal epithelial cells.

In embodiments, the rejuvenated fibroblasts exhibit a transcriptomic profile similar to a transcriptomic profile of young fibroblasts. In embodiments, the rejuvenated fibroblasts exhibit an increased gene expression of one or more nuclear and/or epigenetic markers compared to a reference value as described above. In embodiments, the rejuvenated fibroblasts have a proteolytic activity that is more similar to the proteolytic activity of young cells as described above. In embodiments, the rejuvenated fibroblasts exhibit improved mitochondria health and function compared to a reference value as described above. In embodiments, the rejuvenated fibroblasts exhibit a reversal of the methylation landscape.

In embodiments, the rejuvenated endothelial cells exhibit a transcriptomic profile similar to a transcriptomic profile of young endothelial cells. In embodiments, the rejuvenated endothelial cells exhibit increased gene expression of one or more nuclear and/or epigenetic markers compared to a reference value as described above. In embodiments, the rejuvenated endothelial cells have a proteolytic activity that is more similar to the proteolytic activity of young cells as described above. In embodiments, the rejuvenated endothelial cells exhibit improved mitochondria health and function compared to a reference value as described above. In embodiments, the rejuvenated endothelial cells exhibit a reversal of the methylation landscape.

In embodiments, the rejuvenated chondrocytes exhibit reduced expression of inflammatory factors and/or and increased ATP and collagen metabolism. In embodiments, the inflammatory factors include RANKL, iNOS2, IL6, IFNα, MCP3 and MIP1A. In embodiments, the rejuvenated chondrocytes exhibit reduced expression of RANKL. In embodiments, the rejuvenated chondrocytes exhibit reduced expression of iNOS2. In embodiments, the rejuvenated chondrocytes exhibit reduced expression of IL6. In embodiments, the rejuvenated chondrocytes exhibit reduced expression of IFNα. In embodiments, the rejuvenated chondrocytes exhibit reduced expression of MCP3. In embodiments, the rejuvenated chondrocytes exhibit reduced expression of MIP1A. In embodiments, the rejuvenated chondrocytes exhibit reduced expression of RANKL, iNOS2, IL6, IFNα, MCP3 and MIP1A. In embodiments, the rejuvenated chondrocytes exhibit increased ATP and collagen metabolism. In embodiments, ATP and collagen metabolism is measured by one or more of increased ATP levels, decreased ROS and increased SOD2 expression, increased COL2A1 expression and overall proliferation by the chondrocytes. In embodiments, ATP and collagen metabolism is measured by increased ATP levels. In embodiments, ATP and collagen metabolism is measured by decreased ROS and increased SOD2 expression. In embodiments, ATP and collagen metabolism is measured by increased COL2A1 expression and overall proliferation by the chondrocytes.

In embodiments, the rejuvenated skeletal muscle stem cells exhibit higher proliferative capacity, enhanced ability to differentiate into myoblasts and muscle fibers, restored lower kinetics of activation from quiescence, ability to rejuvenate the muscular microniche, restore youthful force in the muscle, or a combination thereof.

In embodiments, the rejuvenated keratinocytes exhibit higher proliferative capacity, reduced inflammatory phenotype, lower RNAKL and INOS2 expression, reduced expression of cytokines MIP1A, IL6, IFNα, MCP3, increased ATP, increased levels of SOD2 and COL2A1 expression.

In embodiments, the rejuvenated mesenchymal stem cells exhibit reduction in senescence parameters, increased cell proliferation, and/or a decrease in ROS levels. In embodiments, the rejuvenated mesenchymal stem cells exhibit reduction in senescence parameters. In embodiments, the senescence parameters include p16 expression, p21 expression and positive SAβGal staining. In embodiments, the rejuvenated mesenchymal stem cells exhibit increased cell proliferation. In embodiments, the rejuvenated mesenchymal stem cells exhibit a decrease in ROS levels. In embodiments, the rejuvenated mesenchymal stem cells exhibit reduction in senescence parameters, increased cell proliferation, and a decrease in ROS levels.

In embodiments, the rejuvenated conical epithelial cells exhibit a reduction in senescence parameters. In embodiments, the senescence parameters include one or more of expression of p21, expression of p16, mitochondria biogenesis PGC1α, and expression of inflammatory factor IL8. In embodiments, the senescence parameters include p21. In embodiments, the senescence parameters include expression of p16. In embodiments, the senescence parameters include mitochondria biogenesis PGC1α. In embodiments, the senescence parameters include expression of inflammatory factor IL8. In embodiments, the senescence parameters include one expression of p21, expression of p16, mitochondria biogenesis PGC1α, and expression of inflammatory factor IL8.

The methods of the disclosure can be used to rejuvenate cells in culture (e.g., ex vivo or in vitro) to improve function and potency for use in cell therapy. The cells used in treatment of a patient may be autologous or allogeneic. Preferably, the cells are derived from the patient or a matched donor. For example, in ex vivo therapy, cells are obtained directly from the patient to be treated, transfected with mRNAs encoding cellular reprogramming factors, as described herein, and reimplanted in the patient. Such cells can be obtained, for example, from a biopsy or surgical procedure performed on the patient. Alternatively, cells in need of rejuvenation can be transfected directly in vivo with mRNAs encoding cellular reprogramming factors.

In another aspect, a method for inducing proliferation of a cell, such as an immune cell, is provided. In some embodiments, the method comprises exposing the cell to mRNA encoding one or more reprogramming factors, whereby said exposing achieves expression of the one or more reprogramming factors in the cell to enhance the proliferation of the cell, with retention of its identity. In some embodiments, the method for inducing proliferation does not induce exhaustion. In some embodiments, the proliferation results from prevention or reversal of exhaustion.

In another aspect, a method for inducing proliferation is performed before, concurrently, or after a method for inhibiting, preventing, or reversing exhaustion. In some embodiments, a method for inducing proliferation is performed at any time understood by one skilled in the art to provide sufficient proliferation before a method for inhibiting, preventing, or reversing exhaustion. In some embodiments, a method for inducing proliferation is performed at any time understood by one skilled in the art to provide sufficient proliferation after a method for inhibiting, preventing, or reversing exhaustion. In some embodiments, a method for inducing proliferation is performed 1, 2, 3, 4, 5, 6, 7, 8, 9, 10, 11, 12, 13, 14, 15, 16, 17, 18, 19, 20, or 21 days before a method for inhibiting, preventing, or reversing exhaustion. In some embodiments, a method for inducing proliferation is performed 1, 2, 3, 4, 5, 6, 7, 8, 9, 10, 11, 12, 13, 14, 15, 16, 17, 18, 19, 20, or 21 days after a method for inhibiting, preventing, or reversing exhaustion.

In some embodiments, methods of the present technology comprise immune cells that are lymphocytes, granulocytes, monocytes, macrophages, microglia, or dendritic cells. In some embodiments, the lymphocyte is a T-cell, a B-cell or a natural killer (NK) cell. In some embodiments, the lymphocyte is a tumor-infiltrating lymphocyte.

In other embodiments, the lymphocyte is a T-cell. In some embodiments, the T-cell is a cytotoxic T cell (CD8+), a helper T cell (CD4+), a suppressor or regulatory T cell (Treg), a memory T cell, a natural killer T cell (NKT cell), or a gamma delta T cell. In other embodiments, the helper T cell is a Th1, Th2, Th17, Th9, or Tfh T-cell. In some embodiments, the memory T cell is a central memory T cell, an effector memory T cell, a tissue resident memory T cell, or a virtual memory T cell. In some embodiments, suppressor or regulatory T cells of the present technology are FOXP3+ T cells or FOXP3− T cells. In some embodiments, the NKT cell is a subset of CD1d-restricted T cells.

In some embodiments, a granulocyte of the present technology is a neutrophil, an eosinophil, a basophil, or a mast cell.

In other embodiments, a lymphocyte of the present technology is a B-cell. In some embodiments, a B-cell is a memory B-cell or a plasma cell.

In other embodiments, the immune cell is a monocyte, macrophage, microglial cell, or dendritic cell.

In embodiments, the lipids together with the mRNA form a lipid-nanoparticle composition. The lipid-nanoparticle composition can further comprise a helper lipid, a stabilization lipid, and/or a structural lipid. Suitable ionizable lipids, helper lipids, stabilization lipids, structural lipids are described in, for example, U.S. Publication No. 2011/0117125 and in U.S. Pat. Nos. 8,058,069, 9,364,435, 10,703,789, and 11,028,370, the disclosure of lipids therein incorporated by reference herein.

In embodiments, the lipid-nanoparticle composition comprises a phospholipid, and examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, and dilinoleoylphosphatidylcholine.

The lipid-nanoparticle composition in some embodiments may comprise a neutral lipid which is either in an uncharged or neutral zwitterionic form depending on pH. The lipid-nanoparticle composition can also comprise a lipid that is a neutral lipid at physiological pH. Examples include diacylphosphatidylcholine, diacylphosphatidylethanolamine, ceramide, sphingomyelin, cephalin, cholesterol, cerebrosides, and diacylglycerols.

The lipid-nanoparticle composition in some embodiments may comprise an anionic lipid, which refers to any lipid that is negatively charged at physiological pH. These lipids include, but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

The lipid-nanoparticle composition, in some embodiments, may comprise a cationic lipid which refers to any of a lipid species that carries a net positive charge at a selected pH, such as physiological pH (e.g., pH of about 7.0). In an embodiment, cationic lipids comprising alkyl chains with multiple sites of unsaturation, e.g., at least two or three sites of unsaturation, are used to form the lipid particles. Cationic lipids and related analogs are described in U.S. Patent Publication Nos. 2011/0117125, 2006/0083780 and 2006/0240554; U.S. Pat. Nos. 5,208,036; 5,264,618; 5,279,833; 5,283,185; 5,753,613; and 5,785,992; and PCT Publication No. WO 96/10390, the disclosures of which are herein incorporated by reference for disclosure of lipid species. In embodiments, the cationic lipids comprise a protonatable tertiary amine (e.g., pH titratable) head group, C18 alkyl chains, ether linkages between the head group and alkyl chains, and 0 to 3 double bonds. Such lipids include, for example, 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA) and 1,2-dilinolenyloxy-N,N-dimethyl-3-aminopropane (DLenDMA).

The lipid-nanoparticle composition in some embodiments may comprise a neutral a structural lipid, such as cholesterol, fecosterol, sitosterol, campesterol, stigmasterol, brassicasterol, ergosterol, tomatidine, tomatine, ursolic acid and/or alpha-tocopherol.

The lipid-nanoparticle composition may also comprise a polyethylene glycol (PEG) or PEG-modified lipid. Non-limiting examples include PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEG lipid may be PEG-c-DOMG (PEG modified carbamoyl-1,2-dimyristyloxl-propyl-3-amine), PEG-DMG (PEG modified 1,2-dimyristoyl-sn-glycero-3-methoxypolyethylene glycol), PEG-DLPE (PEG modified 1,2-dilauroyl-sn-glycero-3-phosphoethanolamine), PEG-DMPE (PEG modified 1,2-dimyristoyl-sn-glycero-3-phosphoethanolamine), PEG-DPPC (PEG modified 1,2-dipalmitoyl-sn-glycero-3-phosphocholine), or a PEG-DSPE (PEG modified 1,2-distearoyl-sn-glycero-3-phosphoethanolamine-N-[amino(polyethylene glycol)-2000) lipid.

The lipid-nanoparticle composition in some embodiments, may comprise one or more permeability enhancer molecules, carbohydrates, polymers, surface altering agents, or other components. Carbohydrates may include simple sugars, e.g., glucose and polysaccharides, e.g., glycogen and derivatives and analogs thereof.

In some embodiments, lipid nanoparticles or “LNP” are used for delivering the nucleic acids to the cells. As mentioned above, the LNP can comprise natural lipids or synthetic lipids including conjugated lipids or polymers (e.g. PEGylated lipids). The LNPs can comprise any one or more of neutral lipids, zwitterionic, lipids, ionizable lipids, cationic lipids, and anionic lipids. In embodiments, the LNPs comprise natural or synthetic monoacyl or diacyl forms of phosphatidylcholine (PC), phosphatidylglycerol (PG), phosphatidylserine (PS), phosphatidylethanolamine (PE), and phosphatidic acid (PA), or monoacyl, diacyl, triacyl or tetra acyl forms of cardiolipin. In some embodiments, the LNP is a micelle or an inverted micelle (reverse micelle). In other embodiments, the LNP is a unilamellar liposome or a multilamellar liposome.

The cellular aging process has been postulated to be caused by the loss of both genetic and epigenetic information. Loss of genetic information that contributes to cellular aging is typically in the form of genetic mutations such as substitutions, and deletions in an organism's genome. Loss of or changes in epigenetic information associated with cellular aging can take the form of covalent modifications to DNA, such as 5-methylcytosine (5mC), hydroxymethylcytosine (5hmeC), 5-formylcytosine (fC), and 5-carboxylcytosine (caC) and adenine methylation, and to certain proteins, such as lysine acetylation, lysine and arginine methylation, serine and threonine phosphorylation, and lysine ubiquitination and sumoylation of histone proteins. Loss of and changes in the epigenetic information can result in dysregulation of cellular processes, including processes that maintain cell identity, causing cells to exhibit traits that are associated with aging such as senescence.

The methods, compositions, and kits of the present disclosure rejuvenate cells by preventing and reversing the cellular causes of aging. The methods, compositions and kits of the present disclosure rejuvenate cells by restoring epigenetic information that has been lost due to the aging process, injury or disease. The methods, compositions and kits comprise a synthetic, persistent RNA comprising one or more heterologous polynucleotide sequences that encode one or more reprogramming factors. The synthetic, persistent RNA, in an embodiment, is an RNA vector or construct comprising a combination of elements described infra. In an embodiment, the synthetic, persistent RNA is a self-replicating RNA, also referred to as an RNA replicon. In another embodiment, the synthetic, persistent RNA is a circular polyribonucleotide.

In an embodiment, the self-replicating RNA comprises a replicase domain, such as a replicase domain from a virus. The self-replicating RNA encodes for the expression of nonstructural protein genes such that it can direct its own replication (amplification). In embodiments, the RNA replicon comprises, 5′ and 3′ virus replication recognition sequences, coding sequences for virus nonstructural proteins, and/or optionally a polyadenylation tail. It may additionally contain one or more elements, such as an internal ribosome entry site (IRES) sequence, a core or mini-promoter, and the like, to direct the expression, meaning transcription and translation, of a heterologous RNA sequence. The replicon can comprise, in one embodiment, 5′ and 3′ virus replication recognition sequences, coding sequences for a virus nonstructural proteins, optional polyadenylation tail, and one or more of a coding sequences for expression of reprogramming factor(s), such as those described infra.

In one embodiment, the IRES sequence is identical to, based on, derived from a viral, bacterial, eukaryotic, or synthetic IRES sequence. In an embodiment, the IRES sequence has at least about 70%, 75%, 80%, 85%, 86%, 97%, 88%, 89%, 90%, 91%, 92%, 93%, 94%, 95%, 96%, 97%, 98% or 99% sequence identity to a nucleotide sequence of viral, bacterial, eukaryotic, or synthetic origin.

In an embodiment, the replicase domain is a positive-stranded RNA virus replicase domain. In positive-strand RNA viruses the components of the replicase complex are translated directly from the genomic RNA. Viral polypeptides not required for RNA replication, which mainly constitute structural proteins, can either also be translated from the genomic RNA or from one or more subgenomic mRNAs transcribed from a negative sense cRNA template, depending on the specific type of virus. Genomes of members of the group using the former expression strategy contain one long open reading frame (ORF), and include flaviviruses and picornaviruses. The RNA with positive polarity (genome orientation) is translated into one polyprotein that is subsequently processed into the viral proteins. Translation of this RNA leads to a polyprotein that is co-translationally and post translationally processed by viral and host cellular proteases. Viruses that characterized by the subgenomic RNAs used for expression of part of their genes include togaviruses and caliciviruses, which transcribe one RNA of subgenomic length encoding the structural proteins. Coronaviruses and arteriviruses use multiple subgenomic mRNAs for expression of structural and accessory proteins. The replicase genes of these viruses are located in the 5′ part of the genome upstream of the structural genes. For all of these viruses the subgenomic RNAs are 3′ co-terminal with the genomic RNA. Tews and Meyers, RNA Vaccines: Methods and Protocols, Methods in Molecular Biology, Vol 1449, Chapter 2: 2017.

In an embodiment, the replicase domain is comprised of a non-structural replicase domain from a virus, and in an embodiment, the virus an alpha virus. The RNA replicon is, in an embodiment, an alphavirus replicon RNA comprising at least one non-structural replicase domain from an alphavirus and at least one non-alphavirus heterologous sequence encoding factors for a reprogramming factor that when expressed in a somatic cell rejuvenates the cell and/or induces generation of a pluripotent stem cell. In an embodiment, an alphavirus structural protein/protein(s) refers to one or a combination of the structural proteins encoded by alphaviruses. These are produced by the virus as a polyprotein and are represented generally in the literature as C-E3-E2-6k-E1. E3 and 6k serve as membrane translocation/transport signals for the two glycoproteins, E2 and E1. Thus, use of the term E1 herein can refer to E1, E3-E1, 6k-E1, or E3-6k-E1, and use of the term E2 herein can refer to E2, E3-E2, 6k-E2, or E3-6k-E2. Attenuating mutations can be introduced into any one or more of the alphavirus structural proteins.

In an embodiment, the replicon comprises sequences obtained from an alphavirus selected from the group consisting of Eastern Equine Encephalitis virus (EEE), Venezuelan Equine Encephalitis virus (VEE), Everglades virus, Mucambo virus, Pixuna virus Western Equine Encephalitis virus (WEE), Sindbis virus, Semliki Forest virus, Middelburg virus, Chikungunya virus, O'nyong-nyong virus, Ross River virus, Barmah Forest virus, Getah virus, Sagiyama virus, Bebaru virus, Mayaro virus, Una virus, Aura virus, Whataroa virus, Babanki virus, Kyzylagach virus, Highlands J virus, Fort Morgan virus, Ndumu virus and Buggy Creek virus.

Self-replicating constructs are described, for example, in U.S. Patent Publication Nos. 2018/0216079 and 2021/0108179, which are incorporated by reference herein.

As mentioned above, the synthetic, persistent RNA can also be a circular polyribonucleotide. The circular polyribonucleotide, or circular RNA, is a polyribonucleotide that forms a circular structure through covalent or non-covalent bonds. In some embodiments, the circular polyribonucleotide is non-immunogenic in a mammal, e.g., a human. In some embodiments, the circular polyribonucleotide is capable of replicating or replicates in a cell.

In some embodiments, the circular polyribonucleotide comprises a regulatory element, e.g., a sequence that modifies expression of an expression sequence within the circular polyribonucleotide. A regulatory element may include a sequence that is located adjacent to an expression sequence that encodes an expression product. A regulatory element may be linked operatively to the adjacent sequence. A regulatory element may increase an amount of product expressed as compared to an amount of the expressed product when no regulatory element exists. In addition, one regulatory element can increase an amount of products expressed for multiple expression sequences attached in tandem. Hence, one regulatory element can enhance the expression of one or more expression sequences. Multiple regulatory element are well-known to persons of ordinary skill in the art.

In some embodiments, the regulatory element is a translation modulator. A translation modulator can modulate translation of the expression sequence in the circular polyribonucleotide. A translation modulator can be a translation enhancer or suppressor. In some embodiments, the circular polyribonucleotide includes at least one translation modulator adjacent to at least one expression sequence. In some embodiments, the circular polyribonucleotide includes a translation modulator adjacent each expression sequence. In some embodiments, the translation modulator is present on one or both sides of each expression sequence, leading to separation of the expression products, e.g., peptide(s) and or polypeptide (s).

In some embodiments, a translation initiation sequence can function as a regulatory element. In some embodiments, a translation initiation sequence comprises an AUG codon. In some embodiments, a translation initiation sequence comprises any eukaryotic start codon such as AUG, CUG, GUG, UUG, ACG, AUC, AUU, AAG, AUA, or AGG. In some embodiments, a translation initiation sequence comprises a Kozak sequence. In some embodiments, translation begins at an alternative translation initiation sequence, e.g., translation initiation sequence other than AUG codon, under selective conditions, e.g., stress induced conditions. As a non-limiting example, the translation of the circular polyribonucleotide may begin at alternative translation initiation sequence, such as ACG. As another non-limiting example, the circular polyribonucleotide translation may begin at alternative translation initiation sequence, CTG/CUG. As yet another non-limiting example, the circular polyribonucleotide translation may begin at alternative translation initiation sequence, GTG/GUG. As yet another non-limiting example, the circular polyribonucleotide may begin translation at a repeat-associated non-AUG (RAN) sequence, such as an alternative translation initiation sequence that includes short stretches of repetitive RNA e.g. CGG, GGGGCC, CAG, CTG.

Nucleotides flanking a codon that initiates translation, such as, but not limited to, a start codon or an alternative start codon, are known to affect the translation efficiency, the length and/or the structure of the circular polyribonucleotide. Masking any of the nucleotides flanking a codon that initiates translation may be used to alter the position of translation initiation, translation efficiency, length and/or structure of the circular polyribonucleotide.

In one embodiment, a masking agent may be used near the start codon or alternative start codon in order to mask or hide the codon to reduce the probability of translation initiation at the masked start codon or alternative start codon. Non-limiting examples of masking agents include antisense locked nucleic acids (UNA) oligonucleotides and exon-junction complexes (EJCs). (See e.g., Matsuda and Mauro describing masking agents LNA oligonucleotides and EJCs (PLoS ONE, 2010 5: 11)). In another embodiment, a masking agent may be used to mask a start codon of the circular polyribonucleotide in order to increase the likelihood that translation will initiate at an alternative start codon.

In some embodiments, the circular polyribonucleotide encodes a polypeptide and may comprise a translation initiation sequence, e.g., a start codon. In some embodiments, the translation initiation sequence includes a Kozak or Shine-Dalgarno sequence. In some embodiments, the circular polyribonucleotide includes the translation initiation sequence, e.g., Kozak sequence, adjacent to an expression sequence. In some embodiments, the translation initiation sequence is a non-coding start codon. In some embodiments, the translation initiation sequence, e.g., Kozak sequence, is present on one or both sides of each expression sequence, leading to separation of the expression products. In some embodiments, the circular polyribonucleotide includes at least one translation initiation sequence adjacent to an expression sequence. In some embodiments, the translation initiation sequence provides conformational flexibility to the circular polyribonucleotide. In some embodiments, the translation initiation sequence is within a substantially single stranded region of the circular polyribonucleotide.

The circular polyribonucleotide may include more than 1 start codon such as, but not limited to, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 50, at least 60 or more than 60 start codons. Translation may initiate on the first start codon or may initiate downstream of the first start codon.

In some embodiments, the circular polyribonucleotide may initiate at a codon which is not the first start codon, e.g., AUG. Translation of the circular polyribonucleotide may initiate at an alternative translation initiation sequence, such as, but not limited to, ACG, AGG, AAG, CTG/CUG, GTG/GUG, ATA/AUA, ATT/AUU, TTG/UUG. In some embodiments, translation begins at an alternative translation initiation sequence under selective conditions, e.g., stress induced conditions. As a non-limiting example, the translation of the circular polyribonucleotide may begin at alternative translation initiation sequence, such as ACG. As another non-limiting example, the circular polyribonucleotide translation may begin at alternative translation initiation sequence, CTG/CUG. As yet another non-limiting example, the circular polyribonucleotide translation may begin at alternative translation initiation sequence, GTG/GUG. As yet another non-limiting example, the circular polyribonucleotide may begin translation at a repeat-associated non-AUG (RAN) sequence, such as an alternative translation initiation sequence that includes short stretches of repetitive RNA e.g. CGG, GGGGCC, CAG, CTG.

In some embodiments, the circular polyribonucleotide comprises an internal ribosome entry site (IRES) element. A suitable IRES element to include in a circular polyribonucleotide comprises an RNA sequence capable of engaging an eukaryotic ribosome. In one embodiment, the IRES element is derived from the DNA of an organism including, but not limited to, a virus, a bacterium, a eukaryote organism, and a mammal. In an embodiment, the IRES is from a Drosophila species. Viral DNA may be derived from, but is not limited to, picornavirus complementary DNA (cDNA), with encephalomyocarditis virus (EMCV) cDNA and poliovirus cDNA. In one embodiment, Drosophila DNA from which an IRES element is derived includes, but is not limited to, an Antennapedia gene from Drosophila melanogaster.

In some embodiments, the IRES element is at least partially derived from a virus, for instance, it can be derived from a viral IRES element, such as ABPV IGRpred, AEV, ALPV IGRpred, BQCV IGRpred, BVDV1 1-385, BVDV1 29-391, CrPV 5NCR, CrPV IGR, crTMV IREScp, crTMV_IRESmp75, crTMV_IRESmp228, crTMV IREScp, crTMV IREScp, CSFV, CVB3, DCV IGR, EMCV-R, EoPV_5NTR, ERAV_245-961, ERBV_162-920, EV71_1-748, FeLV-Notch2, FMDV type C, GBV-A, GBV-B, GBV-C, gypsy_env, gypsyD5, gypsyD2, HAV HM175, HCV type 1a, HiPVJGRpred, HIV-1, HoCVlJGRpred, HRV-2, IAPVJGRpred, idefix, KBV IGRpred, LINE-1_ORF1_-1O1_to_-1, LINE-1_ORF1_-302_to_-202, LINE-1_ORF2_-138_to_-86, LINE-1_ORF1_-44_to_-1, PSIV IGR, PV type1 Mahoney, PV_type3_Leon, REV-A, RhPV 5NCR, RhPV IGR, SINV 1 IGRpred, SV40 661-830, TMEV, TMV_UI_IRESmp228, TRV 5NTR, TrV IGR, or TSV IGR. In some embodiments, the IRES element is at least partially derived from a cellular IRES, such as AML1/RUNX1, Antp-D, Antp-DE, Antp-CDE, Apaf-1, Apaf-1, AQP4, ATIR var1, ATIR_var2, ATIR_var3, ATIR_var4, BAG1_p36delta236nt, BAG1_p36, BCL2, BiP_-222_-3, C-IAP1 285-1399, c-IAP1 1313-1462, c-jun, c-myc, Cat-1_224, CCND1, DAP5, eIF4G, eIF4GI-ext, eIF4GII, eIF4GII-long, ELG1, ELH, FGF1A, FMR1, Gtx-133-141, Gtx-1-166, Gtx-1-120, Gtx-1-196, hairless, HAP4, HIF1a, hSNM1, Hsp1O1, hsp70, hsp70, Hsp90, IGF2_leader2, Kv1.4_1.2, L-myc, LamB 1-335-1, LEF1, MNT 75-267, MNT 36-160, MTG8a, MYB, MYT2 997-1152, n-MYC, NDST1, NDST2, NDST3, NDST4L, NDST4S, NRF_-653_-17, NtHSF1, ODC1, p27kip1, p53_128-269, PDGF2/c-sis, Pim-1, PITSLRE_p58, Rbm3, reaper, Scamper, TFIID, TIF4631, Ubx_1-966, Ubx_373-961, UNR, Ure2, UtrA, VEGF-A-133-1, XIAP 5-464, XIAP 305-466, or YAP1. In some embodiments, the IRES element comprises a synthetic IRES, for instance, (GAAA)16, (PPT19)4, KMI1, KMI1, KMI2, KMI2, KMIX, XI, or X2.

In some embodiments, the circular polyribonucleotide includes at least one IRES flanking at least one (e.g., 2, 3, 4, 5, 6 or more) expression sequence. In some embodiments, the IRES flanks both sides of at least one (e.g., 2, 3, 4, 5, 6 or more) expression sequence. In some embodiments, the circular polyribonucleotide includes one or more IRES sequences on one or both sides of each expression sequence, leading to separation of the resulting peptide(s) and or polypeptide(s).

The viral, bacterial, eukaryotic, or synthetic IRES sequence can have at least about 70%, 75%, 80%, 85%, 86%, 97%, 88%, 89%, 90%, 91%, 92%, 93%, 94%, 95%, 96%, 97%, 98% or 99% sequence identity to a nucleotide sequence of viral, bacterial, eukaryotic, or synthetic origin.

In one embodiment, the vector generates a monocistronic mRNA or a polycistronic mRNA, wherein the vector is linear or circular. In one embodiment, the vector is an mRNA producing vector that produces mRNA by in vitro transcription of a DNA vector. The DNA vector can be monocistronic or polycistronic (with 2, 3, 4, 5, 6 or more DNA sequences encoding for a reprogramming factor).

In some embodiments, the linear or circular polyribonucleotide includes one or more expression sequences, and each expression sequence may or may not have a termination element. In some embodiments, the circular polyribonucleotide includes one or more expression sequences, and the expression sequences lack a termination element, such that the circular polyribonucleotide is continuously translated. Exclusion of a termination element may result in rolling circle translation or continuous expression of expression product, e.g., peptides or polypeptides, due to lack of ribosome stalling or fall-off. In such an embodiment, rolling circle translation expresses a continuous expression product through each expression sequence. In some other embodiments, a termination element of an expression sequence can be part of a stagger element. In some embodiments, one or more expression sequences in the circular polyribonucleotide comprises a termination element. However, rolling circle translation or expression of a succeeding (e.g., second, third, fourth, fifth, etc.) expression sequence in the circular polyribonucleotide is performed. In such instances, the expression product may fall off the ribosome when the ribosome encounters the termination element, e.g., a stop codon, and terminates translation. In some embodiments, translation is terminated while the ribosome, e.g., at least one subunit of the ribosome, remains in contact with the circular polyribonucleotide.

In some embodiments, the circular polyribonucleotide includes a termination element at the end of one or more expression sequences. In some embodiments, one or more expression sequences comprises two or more termination elements in succession. In such embodiments, translation is terminated and rolling circle translation is terminated. In some embodiments, the ribosome completely disengages with the circular polyribonucleotide. In some such embodiments, production of a succeeding (e.g., second, third, fourth, fifth, etc.) expression sequence in the circular polyribonucleotide may require the ribosome to reengage with the circular polyribonucleotide prior to initiation of translation. Generally, termination elements include an in-frame nucleotide triplet that signals termination of translation, e.g., UAA, UGA, UAG. In some embodiments, one or more termination elements in the circular polyribonucleotide are frame-shifted termination elements, such as but not limited to, off-frame or −1 and +1 shifted reading frames (e.g., hidden stop) that may terminate translation. Frame-shifted termination elements include nucleotide triples, TAA, TAG, and TGA that appear in the second and third reading frames of an expression sequence. Frame-shifted termination elements may be important in preventing misreads of mRNA, which is often detrimental to the cell.

In some embodiments, the circular polyribonucleotide includes at least one stagger element adjacent to an expression sequence. In some embodiments, the circular polyribonucleotide includes a stagger element adjacent to each expression sequence. In some embodiments, the stagger element is present on one or both sides of each expression sequence, leading to separation of the expression products, e.g., peptide(s) and or polypeptide(s). In some embodiments, the stagger element is a portion of the one or more expression sequences. In some embodiments, the circular polyribonucleotide comprises one or more expression sequences, and each of the one or more expression sequences is separated from a succeeding expression sequence by a stagger element on the circular polyribonucleotide. In some embodiments, the stagger element prevents generation of a single polypeptide (a) from two rounds of translation of a single expression sequence or (b) from one or more rounds of translation of two or more expression sequences. In some embodiments, the stagger element is a sequence separate from the one or more expression sequences. In some embodiments, the stagger element comprises a portion of an expression sequence of the one or more expression sequences.

In some embodiments, the circular polyribonucleotide includes a stagger element. To avoid production of a continuous expression product, e.g., peptide or polypeptide, while maintaining rolling circle translation, a stagger element may be included to induce ribosomal pausing during translation. In some embodiments, the stagger element is at 3′ end of at least one of the one or more expression sequences. The stagger element can be configured to stall a ribosome during rolling circle translation of the circular polyribonucleotide. The stagger element may include, but is not limited to a 2A-like, or CHYSEL (cis-acting hydrolase element) sequence. In some embodiments, the stagger element encodes a sequence with a C-terminal consensus sequence that is X1X2X3EX5NPGP, where Xi is absent or G or H, X₂ is absent or D or G, X₃ is D or V or I or S or M, and X₅ is any amino acid. In some embodiments, this sequence comprises a non-conserved sequence of amino-acids with a strong alpha-helical propensity followed by the consensus sequence −D(V/I)ExNPGP, where x=any amino acid. Some non-limiting examples of stagger elements includes GDVESNPGP, GDIEENPGP, VEPNPGP, IETNPGP, GDIESNPGP, GDVELNPGP, GDIETNPGP, GDVENPGP, GDVEENPGP, GDVEQNPGP, IESNPGP, GDIELNPGP, HDIETNPGP, HDVETNPGP, HDVEMNPGP, GDMESNPGP, GDVETNPGP, GDIEQNPGP, and DSEFNPGP.

In some embodiments, the stagger element cleaves an expression product. In some embodiments, the circular polyribonucleotide includes a stagger element adjacent to at least one expression sequence. In some embodiments, the circular polyribonucleotide includes a stagger element after each expression sequence. In some embodiments, the circular polyribonucleotide includes a stagger element is present on one or both sides of each expression sequence, leading to translation of individual peptide(s) and or polypeptide(s) from each expression sequence.

In some embodiments, a stagger element comprises one or more modified nucleotides or unnatural nucleotides that induce ribosomal pausing during translation. Unnatural nucleotides may include peptide nucleic acid (PNA), morpholine and locked nucleic acid (LNA), as well as glycol nucleic acid (GNA) and threose nucleic acid (TNA). Examples such as these are distinguished from naturally occurring DNA or RNA by changes to the backbone of the molecule. Exemplary modifications can include any modification to the sugar, the nucleobase, the inter-nucleoside linkage (e.g. to a linking phosphate/to a phosphodiester linkage/to the phosphodiester backbone), and any combination thereof that can induce ribosomal pausing during translation.

In some embodiments, the stagger element is present in the circular polyribonucleotide in other forms. For example, in some exemplary circular polyribonucleotides, a stagger element comprises a termination element of a first expression sequence in the circular polyribonucleotide, and a nucleotide spacer sequence that separates the termination element from a first translation initiation sequence of an expression succeeding the first expression sequence. In some examples, the first stagger element of the first expression sequence is upstream of (5′ to) a first translation initiation sequence of the expression succeeding the first expression sequence in the circular polyribonucleotide. In some cases, the first expression sequence and the expression sequence succeeding the first expression sequence are two separate expression sequences in the circular polyribonucleotide. The distance between the first stagger element and the first translation initiation sequence can enable continuous translation of the first expression sequence and its succeeding expression sequence. In some embodiments, the first stagger element comprises a termination element and separates an expression product of the first expression sequence from an expression product of its succeeding expression sequences, thereby creating discrete expression products. In some embodiments, the circular polyribonucleotide comprising the first stagger element upstream of the first translation initiation sequence of the succeeding sequence in the circular polyribonucleotide is continuously translated, while a corresponding circular polyribonucleotide comprising a stagger element of a second expression sequence that is upstream of a second translation initiation sequence of an expression sequence succeeding the second expression sequence is not continuously translated. In some embodiments, there is only one expression sequence in the circular polyribonucleotide.

In exemplary circular polyribonucleotides, a stagger element comprises a first termination element of a first expression sequence in the circular polyribonucleotide, and a nucleotide spacer sequence that separates the termination element from a downstream translation initiation sequence. In some embodiments, the first stagger element is upstream of (5′ to) a first translation initiation sequence of the first expression sequence in the circular polyribonucleotide. In some embodiments the distance between the first stagger element and the first translation initiation sequence enables continuous translation of the first expression sequence and any succeeding expression sequences. In some embodiments, the first stagger element separates one round expression product of the first expression sequence from the next round expression product of the first expression sequences, thereby creating discrete expression products. In some embodiments, the circular polyribonucleotide comprising the first stagger element upstream of the first translation initiation sequence of the first expression sequence in the circular polyribonucleotide is continuously translated, while a corresponding circular polyribonucleotide comprising a stagger element upstream of a second translation initiation sequence of a second expression sequence in the corresponding circular polyribonucleotide is not continuously translated.

In some embodiments, the circular polyribonucleotide comprises more than one expression sequence.

In some embodiments, the circular polyribonucleotide comprises untranslated regions (UTRs). UTRs of a genomic region comprising a gene may be transcribed but not translated. In some embodiments, a UTR may be included upstream of the translation initiation sequence of an expression sequence described herein. In some embodiments, a UTR may be included downstream of an expression sequence described herein. In some instances, one UTR for first expression sequence is the same as or continuous with or overlapping with another UTR for a second expression sequence. In some embodiments, the intron is a human intron. In some embodiments, the intron is a full-length human intron, e.g., ZKSCAN1.

In some embodiments, the circular polyribonucleotide comprises a UTR with one or more stretches of Adenosines and Uridines embedded within. These AU rich signatures may increase turnover rates of the expression product.

Introduction, removal or modification of UTR AU rich elements (AREs) may be useful to modulate the stability or immunogenicity of the circular polyribonucleotide. When engineering specific circular polyribonucleotides, one or more copies of an ARE may be introduced to the circular polyribonucleotide and the copies of an ARE may modulate translation and/or production of an expression product. Likewise, AREs may be identified and removed or engineered into the circular polyribonucleotide to modulate the intracellular stability and thus affect translation and production of the resultant protein.

Any UTR from any gene may be incorporated into the respective flanking regions of the circular polyribonucleotide. Multiple wild-type UTRs of any known gene may be utilized. Artificial UTRs which are not variants of wild type genes may be used. The UTRs or portions thereof may be placed in the same orientation as in the transcript from which they were selected or may be altered in orientation or location. Hence a 5 ‘ or 3’ UTR may be inverted, shortened, lengthened, made chimeric with one or more other 5′ UTRs or 3′ UTRs. In one embodiment, a double, triple or quadruple UTR, such as a 5′ or 3′ UTR, may be used, where a “double” UTR is one in which two copies of the same UTR are encoded either in series or substantially in series.

In some embodiments, the circular polyribonucleotide may include a poly-A sequence. In one embodiment, the poly-A sequence is designed relative to the length of the overall circular polyribonucleotide. This design may be based on the length of the coding region, the length of a particular feature or region (such as the first or flanking regions), or based on the length of the ultimate product expressed from the circular polyribonucleotide. In this context, the poly-A sequence may be 10, 20, 30, 40, 50, 60, 70, 80, 90, or 100% greater in length than the circular polyribonucleotide or a feature thereof. The poly-A sequence may also be designed as a fraction of circular polyribonucleotide to which it belongs. In this context, the poly-A sequence may be 10, 20, 30, 40, 50, 60, 70, 80, or 90% or more of the total length of the construct or the total length of the construct minus the poly-A sequence. Further, engineered binding sites and conjugation of circular polyribonucleotide for Poly-A binding protein may enhance expression.

In one embodiment, the circular polyribonucleotide is designed to include a polyA-G quartet. The G-quartet is a cyclic hydrogen bonded array of four guanine nucleotides that can be formed by G-rich sequences in both DNA and RNA. In one embodiment, the G-quartet is incorporated at the end of the poly-A sequence. The resultant circular polyribonucleotide construct is assayed for stability, protein production and/or other parameters including half-life at various time points. In some embodiments, the polyA-G quartet results in protein production equivalent to at least 75% of that seen using a poly-A sequence of 120 nucleotides alone.

In some embodiments, the circular polyribonucleotide comprises a polyA, lacks a polyA, or has a modified polyA to modulate one or more characteristics of the circular polyribonucleotide. In some embodiments, the circular polyribonucleotide lacking a polyA or having modified polyA improves one or more functional characteristics, e.g., immunogenicity, half-life, expression efficiency, etc.

The circular polyribonucleotide can comprise an encryptogen to reduce, evade or avoid the innate immune response of a cell and/or for stability. In some embodiments, 5′ or 3′UTRs can constitute encryptogens in a circular polyribonucleotide. For example, removal or modification of UTR AU rich elements can be useful to modulate the stability or immunogenicity of the circular polyribonucleotide. In some embodiments, removal of modification of AU rich elements in expression sequence, e.g., translatable regions, can be useful to modulate the stability or immunogenicity of the circular polyribonucleotide.

In some embodiments, an encryptogen comprises one or more protein binding sites that enable a protein to bind to the RNA sequence. By engineering protein binding sites into the circular polyribonucleotide, the circular polyribonucleotide may evade or have reduced detection by the host's immune system, have modulated degradation, or modulated translation, by masking the circular polyribonucleotide from components of the host's immune system. In some embodiments, the circular polyribonucleotide comprises at least one immunoprotein binding site, for example to evade immune responses, e.g., CTL responses. In some embodiments, the immunoprotein binding site is a nucleotide sequence that binds to an immunoprotein and aids in masking the circular polyribonucleotide as exogenous.

In some embodiments, an encryptogen comprises one or more modified nucleotides. Exemplary modifications can include any modification to the sugar, the nucleobase, the internucleoside linkage (e.g. to a linking phosphate/to a phosphodiester linkage/to the phosphodiester backbone), and any combination thereof that can prevent or reduce immune response against the circular polyribonucleotide.

In some embodiments, the circular polyribonucleotide and the self-replicating RNA can include one or more modifications as described elsewhere herein to reduce an immune response from the host as compared to the response triggered by a reference compound, e.g. a circular polyribonucleotide lacking the modifications. In particular, the addition of one or more inosines has been shown to discriminate RNA as endogenous versus viral (Yu, Z. et al. (2015) Cell Res. 25, 1283-1284).

The circular polyribonucleotide and the self-replicating RNA may include one or more substitutions, insertions and/or additions, deletions, and covalent modifications with respect to reference sequences. In some embodiments, the circular polyribonucleotide and the self-replicating RNA can include one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc). The one or more post-transcriptional modifications can be any post-transcriptional modification, such as any of the more than one hundred different nucleoside modifications that have been identified in RNA (Rozenski, et al., Nucl Acids Res, 27:196-197 (1999)). In some embodiments, the RNA comprises at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the mRNA comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the mRNA comprises at least one nucleoside selected from the group consisting of 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In some embodiments, mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

The circular polyribonucleotide and the self-replicating RNA may include any useful modification, such as to the sugar, the nucleobase, or the internucleoside linkage (e.g. to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof).

Circular polyribonucleotides are described, for example, in WO2019/118919, WO2021/0161938, and WO2020/252436, each incorporated by reference herein.

### Reprogramming Factors

The reprogramming factor is a protein, for example a transcription factor, that plays a role in changing adult or differentiated cells into pluripotent stem cells. The term “reprogramming factor” further includes any analogue molecule that mimics the function of the factor. In embodiments, the reprogramming factor is a factor from the Oct family, the Sox family, the Klf family, the Myc family, Nanog family, Glis family, or Lin family.

“Oct family” refers to the family of octamer (“Oct”) transcription factors which play a crucial role in maintaining pluripotency. POU5F1 (POU domain, class 5, transcription factor 1) also known as Oct3/4 is one representative of Oct family Exemplary Oct3/4 proteins are the proteins encoded by the murine Oct3/4 gene (GenBank accession number NM_013633) and the human Oct3/4 gene (GenBank accession number NM_002701). The terms “Oct3/4”, “Oct4,” “OCT4,” “Oct4 protein,” “OCT4 protein” and the like thus refer to any of the naturally-occurring forms of the Octomer 4 transcription factor, or variants thereof that maintain Oct4 transcription factor activity (e.g. within at least 50%, 80%, 90% or 100% activity compared to wild type Oct4 as measured by methods known in the art). In some embodiments, variants have at least 85%, 90%, 91%, 92%, 93%, 94%, 95%, 96%, 97%, 98% or 99% amino acid sequence identity across their whole sequence compared to the naturally occurring Oct4 polypeptide. In other embodiments, the Oct4 protein is the protein as identified by the GenBank reference ADW77327.1.

An Oct reprogramming factor refers to any of the naturally-occurring members of octamer family of transcription factors, or variants thereof that maintain transcription factor activity, similar (within at least 50%, 80%, or 90% activity) compared to the closest related naturally occurring family member, or polypeptides comprising at least the DNA-binding domain of the naturally occurring family member, and can further comprise a transcriptional activation domain. Exemplary Oct polypeptides include Oct-1, Oct-2, Oct-3/4, Oct-6, Oct-7, Oct-8, Oct-9, and Oct-11. e.g. Oct3/4 (referred to herein as “Oct4”) contains the POU domain, a 150 amino acid sequence conserved among Pit-1, Oct-1, Oct-2, and uric-86. See, Ryan, A. K. & Rosenfeld, M. G. Genes Dev. 11, 1207-1225 (1997). In some embodiments, variants have at least 85%, 90%, 91%, 92%, 93%, 94%, 95%, 96%, 97%, 98% or 99% amino acid sequence identity across their whole sequence compared to a naturally occurring Oct polypeptide family member such as to those listed above or such as listed in GenBank accession number NP002692.2 (human Oct4) or NP038661.1 (mouse Oct4). Oct polypeptides (e.g., Oct3/4) can be from human, mouse, rat, bovine, porcine, or other animals.

“Sox family” refers to genes that encode for SRY (sex determining region Y)-box 2, also known as SOX2, associated with maintaining pluripotency. Exemplary Sox2 proteins are the proteins encoded by the murine Sox2 gene (GenBank accession number NM_011443) and the human Sox2 gene (GenBank accession number NM_003106). The terms “Sox2,” “SOX2,” “Sox2 protein,” “SOX2 protein” and the like as referred to herein thus includes any of the naturally-occurring forms of the Sox2 transcription factor, or variants thereof that maintain Sox2 transcription factor activity (e.g. within at least 50%, 80%, 90% or 100% activity compared to wild type Sox2 as measured by methods known in the art). In some embodiments, variants have at least 85%, 90%, 91%, 92%, 93%, 94%, 95%, 96%, 97%, 98% or 99% amino acid sequence identity across their whole sequence compared to the naturally occurring Sox2 polypeptide. In other embodiments, the Sox2 protein is the protein as identified by the NCBI reference NP_003097.1.

A Sox reprogramming factor refers to any of the naturally-occurring members of the SRY-related HMG-box (Sox) transcription factors, characterized by the presence of the high-mobility group (HMG) domain, or variants thereof that maintain transcription factor activity similar (within at least 50%, 80%, or 90% activity) compared to the closest related naturally occurring family member, or polypeptides comprising at least the DNA-binding domain of the naturally occurring family member, and can further comprise a transcriptional activation domain. See, e.g., Dang, D. T., et al., Int. J. Biochem. Cell Biol. 32:1103-1121 (2000). Exemplary Sox polypeptides include, e.g., Sox1, Sox-2, Sox3, Sox4, Sox5, Sox6, Sox7, Sox8, Sox9, Sox10, Sox11, Sox12, Sox13, Sox14, Sox15, Sox17, Sox18, Sox-21, and Sox30. In some embodiments, variants have at least 85%, 90%, 91%, 92%, 93%, 94%, 95%, 96%, 97%, 98% or 99% amino acid sequence identity across their whole sequence compared to a naturally occurring Sox polypeptide family member such as to those listed above or such as listed in GenBank accession number CAA83435 (human Sox2). Sox polypeptides (e.g., Sox1, Sox2, Sox3, Sox15, or Sox18) can be from human, mouse, rat, bovine, porcine, or other animals.

“Klf family” refers to Kruppel-like factor 4 or “Klf” genes that encode for Klf4 proteins are the proteins encoded by the murine klf4 gene (GenBank accession number NM_010637) and the human klf4 gene (GenBank accession number NM_004235). The terms “KLF4,” “KLF4 protein” and the like as referred to herein thus includes any of the naturally-occurring forms of the KLF4 transcription factor, or variants thereof that maintain KLF4 transcription factor activity (e.g. within at least 50%, 80%, 90% or 100% activity compared to wild type KLF4 as measured by methods known in the art). In some embodiments, variants have at least 90% amino acid sequence identity across their whole sequence compared to the naturally occurring KLF4 polypeptide. In other embodiments, the KLF4 protein is the protein as identified by the NCBI reference NP_004226.3.

In other embodiments, the Klf reprogramming factor refers to any of the naturally-occurring members of the family of Kruppel-like factors (Klfs), zinc-finger proteins that contain amino acid sequences similar to those of the Drosophila embryonic pattern regulator Kruppel, or variants of the naturally-occurring members that maintain transcription factor activity similar (within at least 50%, 80%, or 90% activity) compared to the closest related naturally occurring family member, or polypeptides comprising at least the DNA-binding domain of the naturally occurring family member, and can further comprise a transcriptional activation domain. See, Dang, D. T., Pevsner, J. & Yang, V. W., Cell Biol. 32, 1103-1121 (2000). Exemplary Klf family members include, Klf1, Klf2, Klf3, Klf-4, Klf5, Klf6, Klf7, Klf8, Klf9, Klf10, Klf11, Klf12, Klf13, Klf14, Klf15, Klf16, and Klf17. In some embodiments, variants have at least 85%, 90%, 91%, 92%, 93%, 94%, 95%, 96%, 97%, 98% or 99% amino acid sequence identity across their whole sequence compared to a naturally occurring Klf polypeptide family member such as to those listed above or such as listed in GenBank accession number CAX16088 (mouse Klf4) or CAX14962 (human Klf4). Klf polypeptides (e.g., Klf1, Klf4, and Klf5) can be from human, mouse, rat, bovine, porcine, or other animals.

Factors of the Myc family refers to factors encoded by myc proto-oncogenes implicated in cancer. Exemplary c-Myc proteins are the proteins encoded by the murine c-myc gene (GenBank accession number NM_010849) and the human c-myc gene (GenBank accession number NM_002467). N-Myc or L-myc was also used as possible reprogramming factor replacing c-Myc. The terms “c-Myc,” C-MYC,” “c-Myc protein”, “C-MYC protein” and the like includes any of the naturally-occurring forms of the c-Myc transcription factor, or variants thereof that maintain c-Myc transcription factor activity (e.g. within at least 50%, 80%, 90% or 100% activity compared to wild type c-Myc as measured by methods known in the art). In some embodiments, variants have at least 85%, 90%, 91%, 92%, 93%, 94%, 95%, 96%, 97%, 98% or 99% amino acid sequence identity across their whole sequence compared to the naturally occurring c-Myc polypeptide. In other embodiments, the c-Myc protein is the protein as identified by the NCBI reference NP_002458.2.

The Myc family of cellular genes is comprised of c-myc, N-myc, and L-myc, and reference to Myc refers any of the naturally-occurring members of the Myc family (see, e.g., Adhikary, S. & Eilers, M. Nat. Rev. Mol. Cell Biol. 6:635-645 (2005)), or variants thereof that maintain transcription factor activity similar (within at least 50%, 80%, or 90% activity) compared to the closest related naturally occurring family member, or polypeptides comprising at least the DNA-binding domain of the naturally occurring family member, and can further comprise a transcriptional activation domain. Exemplary Myc polypeptides include, e.g., c-Myc, N-Myc and L-Myc. In some embodiments, variants have at least 85%, 90%, or 95% amino acid sequence identity across their whole sequence compared to a naturally occurring Myc polypeptide family member, such as to those listed above or such as listed in GenBank accession number CAA25015 (human Myc). Myc polypeptides (e.g., c-Myc) can be from human, mouse, rat, bovine, porcine, or other animals.

The term “Nanog” or “nanog” refers to a transcription factor involved with self-renewal of undifferentiated embryonic stem cells. In humans, this protein is encoded by the NANOG gene. Exemplary nanog is the protein encoded by murine gene (GenBank accession number XM.sub.13 132755) and human Nanog gene (GenBank accession number NM_024865). The term “Nanog” or “nanog” and the like includes any of the naturally-occurring forms of the Nanog transcription factor, or variants thereof that maintain Nanog transcription factor activity (e.g. within at least 50%, 80%, 90% or 100% activity compared to wild type Nanog as measured by methods known in the art). In some embodiments, variants have at least 90% amino acid sequence identity across their whole sequence compared to the naturally occurring Nanog polypeptide. In other embodiments, the Nanog protein is the protein as identified by the NCBI reference NP_079141.

The term “Lin28” or “Lin-28 homolog A” is a protein that is encoded by the LIN28 gene in humans. Exemplary Lin28 is the protein encoded by murine gene (GenBank accession number NM_145833) and human Lin28 gene (GenBank accession number NM_024674). The term “Lin28” or “Lin28 homolog A” and the like as referred to herein thus includes any of the naturally-occurring forms of the Lin28 transcription factor, or variants thereof that maintain Lin28 transcription factor activity (e.g. within at least 50%, 80%, 90% or 100% activity compared to wild type Lin28 as measured by methods known in the art). In some embodiments, variants have at least 90% amino acid sequence identity across their whole sequence compared to the naturally occurring Lin28 polypeptide. In other embodiments, the Lin28 protein is the protein as identified by the NCBI reference NP_078950.

The term “Glis”, “GLIS family zinc finger 1” or “Glis1” is a protein that is encoded by the Glis family of genes in humans (Gene ID: 148979). The term “GLIS family zinc finger 1” or “Glis1” and the like as referred to herein thus includes any of the naturally-occurring forms of the Glis1 transcription factor, or variants thereof that maintain Glis1 transcription factor activity (e.g. within at least 50%, 80%, 90% or 100% activity compared to wild-type Glis1 as measured by methods known in the art). In some embodiments, variants have at least 90% amino acid sequence identity across their whole sequence compared to the naturally occurring Glis1 polypeptide.

Glis1 was identified from a screening of over 1400 transcription factors and is thought to be enriched in unfertilized eggs and embryos at the one cell stage where it can promote direct reprogramming of somatic cells to induced pluripotent stem cells (iPS cells). Glis1 is believed to regulate expression of numerous genes, either positively or negatively, by promoting multiple pro-reprogramming pathways. These pathways are believed to be activated due to the up regulation of the transcription factors N-Myc, Mycl1, c-Myc, Nanog, ESRRB, FOXA2, GATA4, NKX2-5, as well as the other factors used for reprogramming. In some embodiments, Glis1 enhances cellular reprogramming and/or rejuvenation when expressed in combination with other reprogramming factors, such as OCT4, SOX2, KLF4, Glis1, and/or c-MYC. In other embodiments, over expression of Glis1 provides synergistic effects with Nanog in improving reprogramming efficiency. It is believed that Glis1 may interact with Nanog to enhance reprogramming efficiency by stimulating the MET receptor tyrosine kinase and activating the Wingless/Integrated (WNT) signaling pathway.

In some embodiments, the Glis1 reprogramming factor protein/polypeptide provided herein is encoded by optimized polynucleotide sequence of SEQ ID NO: 10. Accordingly, SEQ ID NO: 10 constitutes altered polynucleotide sequences when compared to wild-type Glis1. The altered nucleotide sequences, such as SEQ ID NO: 10, encode, in some embodiments, a more robust Glis1 reprogramming factor that elicits a smaller triggered immune response, is more stable and/or provides a more desirable activity level when compared to proteins or polypeptides corresponding to wild-type nucleotide sequences. In some embodiments, the Glis1 reprogramming factor protein/polypeptide is encoded by a polynucleotide sequence having at least about 80%, about 85%, about 90%, about 91%, about 92%, about 93%, about 94%, about 95%, about 96%, about 97%, about 98%, about 99%, or about 100% sequence identity to SEQ ID NO: 10. In some embodiments, the Glis1 reprogramming factor protein/polypeptide is encoded by a polynucleotide sequence comprising SEQ ID NO: 10. In some embodiments, the Glis1 reprogramming factor protein/polypeptide is encoded by a polynucleotide sequence consisting essentially of SEQ ID NO: 10. In some embodiments, the Glis1 reprogramming factor protein/polypeptide is encoded by a polynucleotide sequence consisting of SEQ ID NO: 10.

In some embodiments, reprogramming factors provided herein comprise T cell optimized factors. In some embodiments, the T cell optimized reprogramming factors protein/polypeptide provided herein are encoded by optimized polynucleotide sequences of SEQ ID NOs: 11-19. Accordingly, SEQ ID NOs: 11-19 constitute altered polynucleotide sequences when compared to wild-type T cell reprogramming factors. The altered nucleotide sequences, such as SEQ ID NOs: 11-19, encode, in some embodiments, a more robust T cell reprogramming factor that elicits a smaller triggered immune response, is more stable and/or provides a more desirable activity level when compared to proteins or polypeptides corresponding to wild-type nucleotide sequences. In some embodiments, the T cell optimized reprogramming factor protein/polypeptide is encoded by a polynucleotide sequence having at least about 80%, about 85%, about 90%, about 91%, about 92%, about 93%, about 94%, about 95%, about 96%, about 97%, about 98%, about 99%, or about 100% sequence identity to any one of the sequences of SEQ ID NOs: 11-19. In some embodiments, the T cell optimized reprogramming factor protein/polypeptide is encoded by a polynucleotide sequence comprising any one of the sequences of SEQ ID NOs: 11-19. In some embodiments, the T cell optimized reprogramming factor protein/polypeptide is encoded by a polynucleotide sequence consisting essentially of any one of the sequences of SEQ ID NOs: 11-19. In some embodiments, the T cell optimized reprogramming factor protein/polypeptide is encoded by a polynucleotide sequence consisting of any one of the sequences of SEQ ID NOs: 11-19.

In some embodiments, the T cell optimized reprogramming factor comprises OCT4MyoD for T-cells (T-OCT4MyoD, SEQ ID NO: 11) or reprogramming factor protein/polypeptide is encoded by a polynucleotide sequence having at least about 80%, about 85%, about 90%, about 95%, about 96%, about 97%, about 98%, about 99%, or about 100% sequence identity to SEQ ID NO: 11. In some embodiments, the T cell optimized reprogramming factor comprises B18R for T cells (T-B18R, SEQ ID NO: 12) or a reprogramming factor protein/polypeptide is encoded by a polynucleotide sequence having at least about 80%, about 85%, about 90%, about 91%, about 92%, about 93%, about 94%, about 95%, about 96%, about 97%, about 98%, about 99%, or about 100% sequence identity to SEQ ID NO: 12. In some embodiments, the T cell optimized reprogramming factor comprises KLF4 for T cells (T-KLF4, SEQ ID NO: 13) or a reprogramming factor protein/polypeptide is encoded by a polynucleotide sequence having at least about 80%, about 85%, about 90%, about 91%, about 92%, about 93%, about 94%, about 95%, about 96%, about 97%, about 98%, about 99%, or about 100% sequence identity to SEQ ID NO: 13. In some embodiments, the T cell optimized reprogramming factor comprises LIN28 for T cells (T-LIN28, SEQ ID NO: 14) or a reprogramming factor protein/polypeptide is encoded by a polynucleotide sequence having at least about 80%, about 85%, about 90%, about 91%, about 92%, about 93%, about 94%, about 95%, about 96%, about 97%, about 98%, about 99%, or about 100% sequence identity to SEQ ID NO: 14. In some embodiments, the T cell optimized reprogramming factor comprises NANOG for T cells (T-NANOG, SEQ ID NO: 15) or a reprogramming factor protein/polypeptide is encoded by a polynucleotide sequence having at least about 80%, about 85%, about 90%, about 91%, about 92%, about 93%, about 94%, about 95%, about 96%, about 97%, about 98%, about 99%, or about 100% sequence identity to SEQ ID NO: 15. In some embodiments, the T cell optimized reprogramming factor comprises OCT4 for T cells (T-OCT4, SEQ ID NO: 16) or a reprogramming factor protein/polypeptide is encoded by a polynucleotide sequence having at least about 80%, about 85%, about 90%, about 91%, about 92%, about 93%, about 94%, about 95%, about 96%, about 97%, about 98%, about 99%, or about 100% sequence identity to SEQ ID NO: 16. In some embodiments, the T cell optimized reprogramming factor comprises SOX2 for T cells (T-SOX2, SEQ ID NO: 17) or a reprogramming factor protein/polypeptide is encoded by a polynucleotide sequence having at least about 80%, about 85%, about 90%, about 91%, about 92%, about 93%, about 94%, about 95%, about 96%, about 97%, about 98%, about 99%, or about 100% sequence identity to SEQ ID NO: 17. In some embodiments, the T cell optimized reprogramming factor comprises cMYC for T-cells (T-cMyc, SEQ ID NO: 18) or a reprogramming factor protein/polypeptide is encoded by a polynucleotide sequence having at least about 80%, about 85%, about 90%, about 91%, about 92%, about 93%, about 94%, about 95%, about 96%, about 97%, about 98%, about 99%, or about 100% sequence identity to SEQ ID NO: 18. In some embodiments, the T cell optimized reprogramming factor comprises GLIS1 for T-cells (T-GLIS1, SEQ ID NO: 19) or a reprogramming factor protein/polypeptide is encoded by a polynucleotide sequence having at least about 80%, about 85%, about 90%, about 91%, about 92%, about 93%, about 94%, about 95%, about 96%, about 97%, about 98%, about 99%, or about 100% sequence identity to SEQ ID NO: 19.

In an embodiment, the synthetic, persistent RNA encodes for expression of a combination of 2, 3, 4, 5, or 6 reprogramming factors. In an embodiment, the reprogramming factors are one or more of Oct4, Klf4, Sox2, c-Myc (or L-myc), Lin28 (or a Lin-28 homolog A) and Glis1. In another embodiment, the reprogramming factors are one or more of Oct4, Klf4, Sox2, c-Myc (or L-myc), Lin28 (or a Lin-28 homolog A), Nanog and Glis1.

In an embodiment, the synthetic, persistent RNA, is an RNA vector that encode for expression of a combination of 1, 2, 3, 4, 5, 6, or more reprogramming factors. In an embodiment, the reprogramming factors are selected from Oct4, Klf4, Sox2, c-Myc (or L-myc), Lin28, Nanog and Glis1. In an embodiment, the reprogramming factors are Oct4, Klf4, Sox2, c-Myc (or L-myc), Lin28 and Nanog. In another embodiment, the reprogramming factors are Oct4, Klf4, Sox2, c-Myc (or L-myc). In an embodiment, the reprogramming factors are Oct4, Klf4, Sox2. In yet another embodiment, the reprogramming factors are Oct4, Sox2, Lin28, Nanog and Glis1.

In embodiments, a pMK expression vector (Life Technologies), containing a polynucleotide sequence of SEQ ID NOs: 1, a polynucleotide sequence of SEQ ID NO: 2, a polynucleotide sequence of SEQ ID NO: 4, an additionally added internal ribosome entry site (IRES)-GFP, 5′ and 3′ UTRs, and linker regions, is provided for expression and generation of corresponding RNA vectors and/or expression of reprogramming factors as described herein.

In embodiments, a pMK expression vector (Life Technologies), containing a polynucleotide sequence of SEQ ID NOs: 5, a polynucleotide sequence of SEQ ID NO: 6, a polynucleotide sequence of SEQ ID NO: 3, an additionally added internal ribosome entry site (IRES)-GFP, 5′ and 3′ UTRs, and linker regions, is provided for expression and generation of corresponding RNA vectors and/or expression of reprogramming factors as described herein.

In embodiments, a T7-VEE-OKS-iM plasmid, as described in PCT/US2013/041980, containing sequences encoding the non-structural proteins (nsP1 to nsP4) for self-replication, the reprogramming factors Oct4, Klf4, Sox2, and cMyc and an additionally added internal ribosome entry site (IRES)-GFP, is provided for expression and generation of corresponding RNA vectors and/or expression of reprogramming factors as described herein.

In embodiments, self-amplifying RNA molecules are provided, wherein the self-amplifying RNA molecules encode reprogramming factors, such as OCT4 (0), SOX2 (S), KLF4 (K), c-MYC (M), LIN28 (L), NANOG (N), and GLIS1 (G) (each molecule encoding a single factor), that are synthesized via in vitro transcription from plasmid DNA and purified. In embodiments, self-amplifying RNA molecules contain 5′ cap, 5′-UTR, alphavirus NSP1-4 genes, a 26 subgenomic promoter, a coding sequence for a reprogramming factor, a 3′ UTR, and a polyA tail. In other conditions, any individual coding sequence and/or any combination selected from O, S, K, L, M, N and G may be included in the self-amplifying RNA. The alphavirus NSP1-4 genes drive intracellular replication of the self-amplifying RNA after transfection. In embodiments, self-amplifying RNA molecules coding different reprogramming factors are mixed to provide an OSKM cocktail, a OSK cocktail, a OSKG cocktail, a OSKMLN cocktail, or cocktails with other combinations of reprogramming factors (see abbreviations above). In embodiments, the reprogramming factor cocktails contain the reprogramming factor-coding RNAs in identical proportions (e.g., 1:1:1:1:1:1 for O:S:K:L:M:N) or with proportions of individual factors adjusted (e.g., 2:1:1:1:1:1 for O:S:K:L:M:N). Such self-amplifying RNA molecules and vectors provide advantages over other standard RNA molecules and vectors.

In embodiments, mRNA molecules encoding the reprogramming factors OCT4 (0), SOX2 (S), KLF4 (K), c-MYC (M), LIN28 (L), NANOG (N), and GLIS1 (G) (each molecule encoding a single factor) as well as mRNA molecules encoding B18R are synthesized via in vitro transcription from plasmid DNA and purified. Each mRNA molecule contains a 5′ cap, 5′-UTR, a coding sequence for a single reprogramming factor or B18R, a 3′ UTR, and a polyA tail. Inclusion of mRNA molecules and vectors encoding B18R provide advantages over other standard RNA expression approaches.

In embodiments, monocistronic self-amplifying RNA molecules encoding the reprogramming factors OCT4 (0), SOX2 (S), KLF4 (K), c-MYC (M), LIN28 (L), NANOG (N), and GLIS1 (G) (each molecule encoding a single factor) are provided, wherein each monocistronic mRNA molecule contains a 5′ cap, a 5′-UTR containing L7Ae regulatory sequence, a coding sequence for a single reprogramming factor, a 3′ UTR, and a polyA tail. In other conditions, polycistronic RNA molecules that each encode more than one factor are used. Such vectors including L7Ae on-off switch mechanisms allow control of expression of the reprograming factors and the ability to “shut off” expression at desired time points, providing advantages in control of expression when compared to standard vectors.

In embodiments, polycistronic RNA molecules encoding the reprogramming factors OCT4 (0), SOX2 (S), KLF4 (K), c-MYC (M), LIN28 (L), NANOG (N), and GLIS1 (G) (each molecule encoding two, three, four, five, or six factors, for example LMK and OSK) are provided wherein each mRNA molecule contains a 5′cap, 5′-UTR, coding sequences for two, three, four, five, or six factors, an IRES element or 2A element before each coding sequence such that each gene has its own IRES or 2A element, a 3′ UTR, and a polyA tail. Polycistronic RNA expression increases the likelihood of all reprogramming factors, or the minimum amount of factors required for effective epigenetic reprogramming, to be present in the same cell, and therefore providing advantages over compared to standard vectors.

In embodiments, circular RNA molecules encoding the reprogramming factors OCT4 (O), SOX2 (S), KLF4 (K), c-MYC (M), LIN28 (L), NANOG (N), and GLIS1 (G) (each molecule encoding a single reprogramming factor) are provided via in vitro transcription from plasmid DNA, circularized, and purified. In embodiments, circular RNA molecules are produced using the Anabena intron-exon splicing strategy which consists of a fused partial intron at one end of the RNA and a partial exon at the other end RNA. In embodiments, use of circular RNA allows fewer transfections to be applied and lower RNA doses to be used when compared to conventional mRNA because of the persistence and lower immunogenicity of the circular RNA.

Cellular age-reversal, or rejuvenating, is achieved by transient overexpression of one or more mRNAs encoding cellular reprogramming factors. Such cellular reprogramming factors may include transcription factors, epigenetic remodelers, or small molecules affecting mitochondrial function, proteolytic activity, heterochromatin levels, histone methylation, nuclear lamina polypeptides, cytokine secretion, or senescence. In embodiments, the cellular reprogramming factors include one or more of OCT4, SOX2, KLF4, c-MYC, LIN28, NANOG, and Glis1. In embodiments, the cellular reprogramming factors are applied in different molar ratios, for example OCT4, SOX2, KLF4, c-MYC, LIN28, Nanog and GLIS1 at molar ratios of a:b:c:d:e:f, wherein a, b, c, d, e, and f can all be the same number (for example, 1:1:1:1:1:1), some the same number and some different numbers (for example, 3:1:1:1:1:1, 2:1:1:1:1:1, 2:2:1:1:1:1, 2:2:2:1:1:1, 2:2:2:2:1:1, 2:2:2:2:2:1, 3:3:3:3:2:2), or all different numbers (for example 6:4:5:3:2:1), and wherein a, b, c, d, e, and f are each 1-7, i.e., 1-7:1-7:1-7:1-7:1-7:1-7 (or 1-7:1-7:1-7:1-7:1-7, 1-7:1-7:1-7:1-7, 1-7:1-7:1-7, 1-7:1-7, or 1-7:1 in the case of combinations with fewer than 6 factors).

In other embodiments, the synthetic, persistent RNA encodes at least two heterologous polynucleotide sequences that encode reprogramming factors. The synthetic, persistent RNA when in the form of a self-replicating RNA comprises, in an embodiment, from 5′ to 3′: (a replicase domains from a virus)-(a promoter)-(a first reprogramming factor)-(a first reprogramming factor separating region)-(a second reprogramming factor)-(a second reprogramming factor separating region)-(optional additional reprogramming factors-optional additional separating regions)-(optional selectable marker)-(virus 3′UTR and/or polyA tail)-(optional selectable marker)-(optional promoter). The reprogramming factors are heterologous polynucleotide sequences which encode for a reprogramming factor. As described above, the reprogramming factor can be selected from the group consisting of Oct polypeptides, Klf polypeptides, Sox polypeptides, Myc polypeptides, Nanog, Lin28 (or a Lin-28 homolog A) and/or GLIS1.

### Methods of Use

The term “age-related disease or condition” refers to any condition, disease, or disorder associated with aging such as, but not limited to, neurodegenerative diseases (e.g., Alzheimer's disease, Parkinson's disease, Huntington's disease, amyotrophic lateral sclerosis, dementia, and stroke), cardiovascular and peripheral vascular diseases (e.g., atherosclerosis, peripheral arterial disease (PAD), hematomas, calcification, thrombosis, embolisms, and aneurysms), eye diseases (e.g., age-related macular degeneration, glaucoma, cataracts, dry eye, diabetic retinopathy, vision loss), dermatologic diseases (dermal atrophy and thinning, elastolysis and skin wrinkling, sebaceous gland hyperplasia or hypoplasia, senile lentigo and other pigmentation abnormalities, graying hair, hair loss or thinning, and chronic skin ulcers), autoimmune diseases (e.g., polymyalgia rheumatica (PMR), giant cell arteritis (GCA), rheumatoid arthritis (RA), crystal arthropathies, and spondyloarthropathy (SPA)), endocrine and metabolic dysfunction (e.g., adult hypopituitarism, hypothyroidism, apathetic thyrotoxicosis, osteoporosis, diabetes mellitus, adrenal insufficiency, various forms of hypogonadism, and endocrine malignancies), musculoskeletal disorders (e.g., arthritis, osteoporosis, myeloma, gout, Paget's disease, bone fractures, bone marrow failure syndrome, ankylosis, diffuse idiopathic skeletal hyperostosis, hematogenous osteomyelitis, muscle atrophy, peripheral neuropathy, multiple sclerosis, amyotrophic lateral sclerosis (ALS), Duchenne muscular dystrophy, primary lateral sclerosis, and myasthenia gravis), diseases of the digestive system (e.g., liver cirrhosis, liver fibrosis, Barrett's esophagus), respiratory diseases (e.g., pulmonary fibrosis, chronic obstructive pulmonary disease, asthma, chronic bronchitis, pulmonary embolism (PE), lung cancer, and infections), conditions associated with cellular proliferation, and any other diseases and disorders associated with aging.

As used herein, the term “disease or disorder involving cartilage degeneration” is any disease or disorder involving cartilage and/or joint degeneration. The term “disease or disorder involving cartilage degeneration” includes conditions, disorders, syndromes, diseases, and injuries that affect spinal discs or joints (e.g., articular joints) in animals, including humans, and includes, but is not limited to, arthritis, chondroplasia, spondyloarthropathy, ankylosing spondylitis, lupus erythematosus, relapsing polychondritis, and Sjogren's syndrome.

As used herein, the term “muscle degeneration disease or disorder” is any disease or disorder involving muscle degeneration. The term includes conditions, disorders, syndromes, diseases, and injuries that affect muscle tissue such as, but not limited to, muscle atrophy, muscle disuse, muscle tears, burns, surgery, peripheral neuropathy, multiple sclerosis, amyotrophic lateral sclerosis (ALS), Duchenne muscular dystrophy, primary lateral sclerosis, myasthenia gravis, cancer, AIDS, congestive heart failure, chronic obstructive pulmonary disease (COPD), liver disease, renal failure, eating disorders, malnutrition, starvation, infections, or treatment with glucocorticoids.

Conditions associated with cellular proliferation refers to a disease that occurs due to abnormal growth or extension by the multiplication of cells (Walker, Cambridge Dictionary of Biology, Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

“Cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macro globulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

By “therapeutically effective dose or amount” is intended an amount of rejuvenated cells or intracellular expression of the one or more reprograming factors that brings about a positive therapeutic response in a subject in need of tissue repair or regeneration, such as an amount that restores function and/or results in the generation of new tissue at a treatment site. The rejuvenated cells may be produced by transfection in vitro, ex vivo, or in vivo with the synthetic, persistent RNA, for expression of the one or more reprogramming nucleotide sequences encoding one or more cellular reprogramming factors, as described herein. Thus, for example, a “positive therapeutic response” would be an improvement in the age-related disease or condition in association with the therapy, and/or an improvement in one or more symptoms of the age-related disease or condition in association with the therapy, such as restored tissue functionality, reduced pain, improved stamina, increased strength, increased mobility, and/or improved cognitive function. The exact amount (of cells or mRNA) required will vary from subject to subject, depending on the species, age, and general condition of the subject, the severity of the condition being treated, mode of administration, and the like. An appropriate “effective” amount in any individual case may be determined by one of ordinary skill in the art using routine experimentation, based upon the information provided herein.

For example, a therapeutically effective dose or amount of rejuvenated chondrocytes is intended an amount that, when administered as described herein, brings about a positive therapeutic response in a subject having cartilage damage or loss, such as an amount that results in the generation of new cartilage at a treatment site (e.g., a damaged joint). For example, a therapeutically effective dose or amount could be used to treat cartilage damage or loss resulting from a traumatic injury or a degenerative disease, such as arthritis or other disease involving cartilage degeneration. Preferably, a therapeutically effective amount restores function and/or relieves pain and inflammation associated with cartilage damage or loss.

In another example, a therapeutically effective dose or amount of rejuvenated skeletal muscle stem cells is intended an amount that, when administered as described herein, brings about a positive therapeutic response in a subject having muscle damage or loss, such as an amount that results in the generation of new myofibers at a treatment site (e.g., a damaged muscle). For example, a therapeutically effective dose or amount could be used to treat muscle damage or loss resulting from a traumatic injury or a disease or disorder involving muscle degeneration. Preferably, a therapeutically effective amount improves muscle strength and function.

In some embodiments, the methods of the present technology comprise exposing (contacting) a cell, such as an immune cell, to RNA for a dosing interval understood by one of ordinary skill in the art to rejuvenate the cell without resulting in a loss of identity or differentiation. In some embodiments, the methods of the present technology comprise exposing a cell to RNA for a dosing interval of not more than 25, 24, 23, 22, 21, 20, 19, 18, 17, 16, 15, 14, 13, 12, 11, 10, 9, 8, 7, 6, 5, 4, or 3 consecutive days. In some embodiments, the RNA dosing, such as mRNA dosing, is performed at least once daily during the dosing interval. In some embodiments, the RNA dosing is performed less frequently than once per day during the dosing interval, for example once every two days, once every three days, once every four days, once every x days, where x is a number from 4 to 25. Thus, in such embodiments, for example, dosing RNA once every 5 days in a 5 day dosing interval means that the RNA is dosed once in the interval, i.e., once in the total treatment period of 5 days, whereas dosing RNA twice daily in a 5 day dosing interval means that the RNA is dosed 10 times in the interval, i.e., 10 times in the 5 days. In some embodiments, the methods of the present technology comprise exposing a cell to RNA for not more than 21, 18, 14, 10, 7, or 5 consecutive days. In some embodiments, the methods of the present technology comprise exposing a cell to RNA for not more than 18 consecutive days. In some embodiments, the methods of the present technology comprise exposing a cell to RNA for not more than 14 consecutive days. In some embodiments, the methods of the present technology comprise exposing a cell to RNA for not more than 10 consecutive days. In some embodiments, the methods of the present technology comprise exposing a cell to RNA at least once daily for not more than 5 consecutive days. In other embodiments, said exposing (contacting) comprises interrupting said exposing and repeating said exposing after said interrupting. In some embodiments, said exposing (contacting) comprises exposing the cell to RNA for between about 2-5 consecutive days, between about 5-7 consecutive days, between about 7-10 consecutive days, between about 10-12 consecutive days, between about 12-14 consecutive days, between about 14-17 consecutive days, between about 17-19 consecutive, or between about 19-21 consecutive days and in some embodiments, further comprising interrupting said exposing and repeating said exposing after said interrupting. In some embodiments, the duration of exposure is controlled by the mechanisms described herein, e.g., use of self-amplifying RNA, circular RNA, B18R and other decoys, and/or on/off switches. In some embodiments, said repeating is performed any number of times, for example 1, 2, 3, 4, 5, 6, 7, 8, 9, or 10 times, or up to 20 times, or up to 30 times, or more. For in vivo applications, said repeating may continue for any duration of time, for example until a disease is successfully treated or cured, or throughout the life of a subject or patient. In some embodiments, said repeating is performed any time after said interrupting, for example 0, 1, 2, 3, 4, 5, 6, 7, 8, 9, or 10 days, up to 20 days, up to 30 days, up to 3 months, up to 6 months, or up to 1 year after said interrupting. One exposure period is considered to be a dosing interval, such that, for example, a sequence of exposure-interruption-repeat exposure contains two dosing intervals.

In some embodiments, exposing comprises providing a composition comprising the mRNA, wherein the composition comprises an excipient for transfection. In some embodiments, said composition comprises a lipid and the mRNA are associated with the lipid. In some embodiments, the lipids comprise ionizable lipids that can be used in combination with other lipid components, such as helper lipids, stabilization lipids and structural lipids. In some embodiments, the disclosure also provides lipid-nanoparticle compositions comprising such lipids towards delivery of therapeutic nucleic acids. In other embodiments, the composition comprises a polymer and the mRNA are associated with the polymer. In some embodiments, the polymer is a charge-altering releasable transporter. In some embodiments, the charge-altering releasable transporter is at least one of the “block CARTs” or “stat CARTs” described in McKinlay et al. 2017 (PNAS Jan. 24, 2017 114 (4) E448-E456), McKinlay et al. 2018 (PNAS Jun. 26, 2018 115 (26) E5859-E5866), or Haabeth et al. 2018 (PNAS Sep. 25, 2018 115 (39) E9153-E9161), incorporated herein by reference. In some embodiments, the polymer or lipid forms a nanoparticle. In other embodiments, said composition comprises both a polymer and lipid and the mRNA are associated with the polymer and/or the lipid. In some embodiments, the use of a lipid or polymer for delivery of the mRNA, such as in a lipid nanoparticle, polymer nanoparticle, or hybrid lipid-polymer nanoparticle, results in enhanced rejuvenation, proliferation, recovery from or prevention of exhaustion, anti-pathogenic effects, anti-cancer effects, or anti-inflammatory effects in the exposed immune cell compared to using a different delivery mechanism for the mRNA. In some embodiments, the use of a lipid or polymer for delivery of the mRNA results in enhanced rejuvenation, proliferation, recovery from or prevention of exhaustion, anti-pathogenic effects, anti-cancer effects, or anti-inflammatory effects in the exposed immune cell compared to using a different delivery mechanism for the mRNA due to lower toxicity and/or lower physiological impact on the cell when compared to the different delivery mechanism. In some embodiments, the different delivery mechanism is electroporation such that the use of a lipid or polymer, including lipid or polymer nanoparticles, for delivery of the mRNA results in enhanced rejuvenation, proliferation, recovery from or prevention of exhaustion, anti-pathogenic effects, anti-cancer effects, or anti-inflammatory effects in the exposed immune cell compared to when using electroporation. This improvement compared to electroporation can result from reduced toxicity or reduced physiological impact on the cell compared to electroporation.

As used herein, the terms “subject,” “individual,” and “patient,” are used interchangeably herein and refer to any vertebrate subject, including, without limitation, humans and other primates, including non-human primates such as chimpanzees and other apes and monkey species; farm animals such as cattle, sheep, pigs, goats and horses; domestic mammals such as dogs and cats; rodents such as mice, rats, rabbits, hamsters, and guinea pigs; birds, including domestic, wild and game birds such as chickens, turkeys and other gallinaceous birds, ducks, geese, and the like. In some cases, the methods of the disclosure find use in experimental animals, in veterinary application, and in the development of animal models for disease. The term does not denote a particular age. Thus, both adult and newborn individuals are intended to be covered.

From the foregoing, it is appreciated that by using a self-replicating backbone of a virus (the structural genes being removed), such as an alphavirus, to express the reprogramming factors requires a reduced number of transfections (dosings), preferably 1, 2, 3 or 4, into primary human somatic cells to express the reprogramming factors for a therapeutic, rejuvenative effect on the cells. The generation of the alphavirus RF-RNA transcript utilizes a SP6 (or T7) in vitro transcription kit that does not require special conditions and thereby, further simplifies the approach for broad use. By expressing the one, two, three, four, five or six reprogramming factors at consistent, high levels over time in the same cell combined with replication of the virus-reprogramming factor RNA construct for a desired number of multiple cell generations, the virus-reprogramming factor RNA construct approach solves both of the major inefficiency problems associated with repeated daily transfections of four individual reprogramming factor mRNAs. The virus-reprogramming factor RNA construct is an ectopic approach that does not utilize a DNA intermediate and therefore, there is no opportunity for integrative mutation that can occur with DNA vector-based approaches. In addition, the approach can be engineered to express alternative reprogramming factor combinations and/or insertion of additional reprogramming factor ORFs into the reprogramming factor-RNA backbone.

It can also be appreciated that using a circular polyribonucleotide to express the one or more reprogramming factors achieves a therapeutic, rejuvenative effect on the cells with a reduced number of transfections (dosings), preferably 1, 2, 3 or 4. The circular polyribonucleotide expresses the one, two, three, four, five or six reprogramming factors at consistent, high levels over time in the same cell or tissue, to achieve a rejuvenative effect with minimal dosings.

The vectors additionally and optionally include a mechanism to control expression of the one or more reprogramming factors. For example, a mechanism to turn off, silence, cease or curtail expression of one, two, three, four, five, or all of the one or more reprogramming factors after expression for a first period of time can be incorporated into the vector or its environment of use. For example, the vector can include a mechanism that silences the expression of one, a portion or all of the one or more reprogramming factors. This optional embodiment of a mechanism is useful for certain methods of treatment, such as methods involved with cell rejuvenation with retention of cellular identity. Silencing, ceasing or curtailing expression of the one or more reprogramming factors permits generation of a rejuvenated cell, tissue or organ with retention of cellular identity. In an embodiment, the mechanism to silence expression is a mechanism capable of and/or configured to control expression by silencing expression in response to one or more triggers and initiating expression in response to one or more triggers. The mechanism in the vector is, in an embodiment, configured as an on/off switch of expression of the one or more reprogramming factors.

### III. Examples

The following examples are illustrative in nature and are in no way intended to be limiting.

### Example 1

### Self-Replicating RNA (srRNA)

A T7-VEE-OKS-iM plasmid, as described in PCT/US2013/041980, containing sequences encoding the non-structural proteins (nsP1 to nsP4) for self-replication, the reprogramming factors Oct4, Klf4, Sox2, and c-Myc and an additionally added internal ribosome entry site (IRES)-GFP is amplified in E. coli and plasmids are isolated using QIAPrep® (Qiagen, Hilden, Germany). After the linearization with MluI restriction enzyme (Thermo Fisher Scientific), 10 μg template DNA is transcribed in vitro using RiboMAX™ large-scale production system T7 Kit (Promega, Madison, Wis., USA) according to the manufacturer's instructions. Afterwards, 2 U TURBO™ DNase is added for 15 min at 37° C. For 5′-end capping, ScriptCap™ Cap1 Capping System is used followed by 30-end polyadenylation with A-Plus Poly(A) Polymerase Tailing Kit (both from Cellscript, Madison, Wis., USA) according to the manufacturer's instructions. Following each reaction step, srRNA is purified using RNeasy® Kit (Qiagen). The specific lengths of the generated DNA and srRNA products are analyzed using 1% agarose gel electrophoresis.

### Example 2

### Self-Replicating RNA Constructs

A Simplicon™ RNA reprogramming system that uses a single synthetic, polycistronic self-replicating RNA strand is obtained, where the single RNA strand contains the four reprogramming factors, OCT-4, KLF-4, SOX-2 and c-MYC.

Human foreskin fibroblasts are plated in each well of a 6-well plate in low serum fibroblast medium and allowed to attach overnight. The cells are pretreated with B18R growth factor (200 ng/mL) for 2 h at 37° C. and 5% CO₂. The cells are then transfected with 1 μg of Simplicon™ VEE-OKSM-iG and B18r RNA in 2.5 μL of RiboJuice™ mRNA transfection reagent following the manufacturer's protocol. The mixture of Simplicon™ RNA and transfection reagent is incubated at 37° C., 5% CO₂ for 3 h. Following transfection with RNA, medium is exchanged with 2 mL/well of ADMEM medium containing 10% fetal bovine serum (FBS), 1% Gluta-MAX™ supplement and B18R protein (200 ng/mL).

Starting the day after transfection, cells are fed daily with ADMEM with 10% FBS, 1% GlutaMAX™ supplement, B18R protein and 0.5 μg/mL puromycin for a total of 5 days.

### Example 3

### In Vitro Production of Circular RNA

Unmodified linear RNA is synthesized by in vitro transcription using T7 RNA polymerase from a DNA segment having 5′- and 3′-ZKSCAN1 introns and an open reading frame (ORF) encoding green fluorescent protein (GFP) linked to stagger element sequences. Transcribed RNA is purified with an RNA purification system (QIAGEN), treated with alkaline phosphatase (ThermoFisher Scientific, EF0652) following the manufacturer's instructions, and purified again with the RNA purification system.

Splint ligation circular RNA is generated by treatment of the transcribed linear RNA and a DNA splint using T4 DNA ligase (New England Bio, Inc., M0202M), and the circular RNA is isolated following enrichment with RNase R treatment. RNA quality is assessed by agarose gel or through automated electrophoresis (Agilent).

### Example 4

### Self-Amplifying RNA for Expression of Reprogramming Factors

Self-amplifying RNA molecules encoding the reprogramming factors OCT4 (0), SOX2 (S), KLF4 (K), c-MYC (M), LIN28 (L), NANOG (N), and GLIS1 (G) (each molecule encoding a single factor) are synthesized via in vitro transcription from plasmid DNA and purified. Each self-amplifying RNA molecule contains a 5′ cap, 5′-UTR, alphavirus NSP1-4 genes, a 26 subgenomic promoter, a coding sequence for a reprogramming factor, a 3′ UTR, and a polyA tail. In other conditions, any individual coding sequence and/or any combination selected from O, S, K, L, M, N and G may be included in the self-amplifying RNA. The alphavirus NSP1-4 genes drive intracellular replication of the self-amplifying RNA after transfection. Self-amplifying RNA molecules coding different reprogramming factors are then mixed to provide an OSKM cocktail, a OSK cocktail, a OSKG cocktail, a OSKMLN cocktail, or cocktails with other combinations of reprogramming factors (see abbreviations above). The reprogramming factor cocktails contain the reprogramming factor-coding RNAs in identical proportions (e.g., 1:1:1:1:1:1 for O:S:K:L:M:N) or with proportions of individual factors adjusted (e.g., 2:1:1:1:1:1 for O:S:K:L:M:N). As a control, conventional mRNA molecules each encoding a single reprogramming factor are also synthesized via in vitro transcription from plasmid DNA, purified, and mixed to form cocktails.

Human fibroblasts are obtained from Lonza and cultured in Fibroblast Growth Medium-2 (FGM™-2). An aging model is induced in the fibroblasts through treatment with TGF-beta at a concentration of 0.1 to 20 ng/ml for 3 days (“aged”). Untreated fibroblasts are used as a control (“control”). The model is described in detail in Juhl et al. (Scientific Reports volume 10, Article number: 17300 (2020)), incorporated herein by reference.

In other conditions, human fibroblasts from aged donors (for example, >65 years; “aged”) or young donors (for example, <25 years; “control”) are used. For example, neonatal human fibroblasts from newborn (“control”) and old human fibroblasts from 60+ year old (“aged”) are purchased from commercial manufacturer (Lonza). Gene and protein expression profiles are analyzed in “aged” cells that have been treated with reprogramming factors. Reprogramming factor treated “aged” cells exhibit gene and protein expression profiles skewed towards expression profiles seen in “control” cells. For instance, the expression profile of “aged” cells treated with reprogramming factors is shifted towards expression patterns that resemble the expression profiles of “control” cells when compared to the expression profiles of untreated “aged” cells.

For transfection, “aged” and “control” fibroblasts are seeded in 6-well plates at a density of 0.25×10⁶ cells/well and allowed to grow to 70% confluency in FGM™-2. Self-amplifying RNA molecules are prepared as naked RNA in nuclease-free water and then mixed together to provide reprogramming factor combinations of OSKMLN, OSKM, OSK, and OSKG. In other conditions, any individual coding sequence and/or any combination selected from O, S, K, L, M, N and G may be included in the self-amplifying RNA. mRNA molecules are similarly prepared and mixed to provide the same factor combinations. Self-amplifying RNA multifactor cocktails prepared in this manner are mixed with Lipofectamine™ MessengerMAX™ (ThermoFisher) at a ratio of 1:1 to form transfection complexes per manufacturer's instructions. Multifactor mRNA cocktails are similarly mixed with Lipofectamine™ MessengerMAX™ to form transfection complexes. The self-amplifying RNA transfection complexes are then added to the wells containing “aged” and “control” fibroblasts at doses of 5000 ng RNA per well, and transfection is allowed to proceed for 6 hours. Wells receiving mRNA transfection complexes serve as a control. After transfection is complete, the transfection medium is discarded, and fresh medium is applied to the wells. Self-amplifying RNA is transfected once, on Day 1 at the beginning of the experiment. Conventional mRNA is transfected every day.

At 3, 4, 5, 6, and/or 7 days, cell viability and/or proliferation is evaluated using cell proliferation assays (WST-8 or MTT) per the manufacturer's instructions (Sigma Aldrich).

At 3, 4, 5, 6, and/or 7 days, cells are stained with specific antibodies and imaged using confocal microscopy to assess expression of collagen IV, fibronectin, and laminin as rejuvenation markers; vimentin as an aging marker; interferon induced protein with tetratricopeptide repeats 1 (IFIT1), IFIT2, IFIT3, IL6, interferon beta (IFNB) 2′-5′-oligoadenylate synthetase 1 (OAS1), protein kinase R (PKR), and Toll Like Receptor (TLR3) as cellular immune response markers. Lactate dehydrogenase (LDH) assay and Adenylate Kinase (AK) assay are used to measure toxicity following manufacturer's instructions.

At 3, 4, 5, 6, and/or 7 days, cells are lysed, total RNA collected and reverse-transcribed to cDNA. Real-time PCR is used to assess the expression of collagen IV, fibronectin, and laminin as rejuvenation markers; vimentin as an aging marker; IFIT1, IFIT2, IFIT3, IL6, IFNB, OAS1, PKR and TLR3 as cellular immune response markers. LDH assay and AK assay are used to measure toxicity following manufacturer's instructions.

Use of self-amplifying RNA allows fewer transfections to be applied and lower RNA doses to be used when compared to conventional mRNA because of the continued propagation of the self-amplifying RNA. Fewer transfections and lower RNA dose also lead to lower toxicity and as a result to higher reprogramming efficacy, and stronger cellular rejuvenation effects. Therefore, as compared to conventional mRNA, self-amplifying RNA improves cell viability and proliferation, with upregulation of cell rejuvenation markers, and downregulation of cell immune response, toxicity, and aging markers

### Example 5

### Co-Expression of b18r and Reprogramming Factors

mRNA molecules encoding the reprogramming factors OCT4 (0), SOX2 (S), KLF4 (K), c-MYC (M), LIN28 (L), NANOG (N), and GLIS1 (G) (each molecule encoding a single factor) as well as mRNA molecules encoding b18r are synthesized via in vitro transcription from plasmid DNA and purified. Each mRNA molecule contains a 5′ cap, 5′-UTR, a coding sequence for a single reprogramming factor or b18r, a 3′ UTR, and a polyA tail.

Human fibroblasts are obtained from Lonza and cultured in FGM™-2 medium. An aging model is induced in the fibroblasts through treatment with TGF-beta at a concentration of 0.1-20 ng/ml for 3 days (“aged”). Untreated fibroblasts are used as a control (“control”). The model is described in detail in Juhl et al. (Scientific Reports volume 10, Article number: 17300 (2020)), incorporated herein by reference.

In other conditions, human fibroblasts from aged donors (for example, >65 years; “aged”) or young donors (for example, <25 years; “control”) are used. For example, neonatal human fibroblasts from newborn (“control”) and old human fibroblasts from 60+ year old (“aged”) are purchased from commercial manufacturer (Lonza). Gene and protein expression profiles are analyzed in “aged” cells that have been treated with reprogramming factors. Reprogramming factor treated “aged” cells exhibit gene and protein expression profiles skewed towards expression profiles seen in “control” cells. For instance, the expression profile of “aged” cells treated with reprogramming factors is shifted towards expression patterns that resemble the expression profiles of “control” cells when compared to the expression profiles of untreated “aged” cells.

For transfection, “aged” and “control” fibroblasts are seeded in 6-well plates at a density of 0.25×10⁶ cells/well and allowed to grow to 70% confluency in FGM™-2 medium. mRNA molecules coding different reprogramming factors are prepared as naked RNA in nuclease-free H₂O and then mixed together to provide an OSKM cocktail, a OSK cocktail, a OSKG cocktail, a OSKMLN cocktail, or cocktails with other combinations of reprogramming factors (see abbreviations above). The reprogramming factor cocktails contain the reprogramming factor-coding mRNAs in identical proportions (e.g., 1:1:1:1:1:1 for O:S:K:L:M:N) or with proportions of individual factors adjusted (e.g., 2:1:1:1:1:1 for O:S:K:L:M:N). When the cocktails are prepared, mRNA encoding b18r is added to provide combinations such as OSKMLN cocktail+b18r, OSKM cocktail+b18r, OSK cocktail+b18r, and OSKG cocktail+b18r. As a control, combinations of mRNA encoding reprogramming factors without the mRNA encoding b18r, e.g., OSKMLN, OSKM, OSK, and OSKG, are used. mRNA cocktails prepared in this manner are mixed with Lipofectamine™ MessengerMAX™ at a ratio of 1:1 to form transfection complexes per manufacturer's instructions. The mRNA transfection complexes are then added to the wells containing “aged” and “control” fibroblasts at doses of 5000 ng RNA per well, and transfection is allowed to proceed for 6 hours. Wells receiving reprogramming factor mRNA transfection complexes without the mRNA encoding b18r serve as control. After transfection is complete, the transfection medium is discarded, and fresh medium applied to the wells. mRNA is transfected every day, every other day, every three days, every four days, or every five days.

At 3, 4, 5, 6, and/or 7 days, cell viability and/or proliferation is evaluated using WST-8 or MTT assay per the manufacturer's instructions (Sigma Aldrich).

At 3, 4, 5, 6, and/or 7 days, cells are stained (immunofluorescence) to evaluate the expression of collagen IV, fibronectin, and laminin as rejuvenation markers; vimentin as an aging marker; IFIT1, IFIT2, IFIT3, IL6, IFNB, OAS1, PKR and TLR3 as cellular immune response markers. LDH assay and AK assay are used to measure toxicity following manufacturer's instructions.

At 3, 4, 5, 6, and/or 7 days, cells are lysed, total RNA collected and reverse-transcribed to cDNA. Real-time PCR is used to evaluate expression of collagen IV, fibronectin, and laminin as rejuvenation markers; vimentin as an aging marker; IFIT1, IFIT2, IFIT3, IL6, IFNB, OAS1, PKR and TLR3 as cellular immune response markers. LDH assay and AK assay are used to measure toxicity following manufacturer's instructions.

Addition of mRNA encoding b18r results in higher translation efficiency and lower toxicity due to reduced type I interferon response as well as the need for fewer transfections and lower mRNA doses. This leads to higher reprogramming efficacy and stronger cellular rejuvenation effects. Thus, compared to treatments in the absence of b18r mRNA, addition of mRNA encoding b18r results in higher cell viability and proliferation, with upregulation of cell rejuvenation markers, and downregulation of cell immune response, toxicity, and aging markers.

### Example 6

### Vectors with On-Off Switch for Expression of Reprogramming Factors

Monocistronic self-amplifying RNA molecules encoding the reprogramming factors OCT4 (0), SOX2 (S), KLF4 (K), c-MYC (M), LIN28 (L), NANOG (N), and GLIS1 (G) (each molecule encoding a single factor) are synthesized via in vitro transcription from plasmid DNA and purified. Each monocistronic mRNA molecule contains a 5′ cap, a 5′-UTR containing L7Ae regulatory sequence, a coding sequence for a single reprogramming factor, a 3′ UTR, and a polyA tail. In other conditions, polycistronic RNA molecules each encoding more than one factor are used.

Human fibroblasts are obtained from Lonza and cultured in FGM™-2 medium. An aging model is induced in the fibroblasts through treatment with TGF-beta at a concentration of 0.1-20 ng/ml for 3 days (“aged”). Untreated fibroblasts are used as a control (“control”). The model is described in detail in Juhl et al. (Scientific Reports volume 10, Article number: 17300 (2020)), incorporated herein by reference.

In other conditions, human fibroblasts from aged donors (for example, >65 years; “aged”) or young donors (for example, <25 years; “control”) are used. For example, neonatal human fibroblasts from newborn (“control”) and old human fibroblasts from 60+ year old (“aged”) are purchased from commercial manufacturer (Lonza). Gene and protein expression profiles are analyzed in “aged” cells that have been treated with reprogramming factors. Reprogramming factor treated “aged” cells exhibit gene and protein expression profiles skewed towards expression profiles seen in “control” cells. For instance, the expression profile of “aged” cells treated with reprogramming factors is shifted towards expression patterns that resemble the expression profiles of “control” cells when compared to the expression profiles of untreated “aged” cells.

For transfection, “aged” and “control” fibroblasts are seeded in 6-well plates at a density of 0.25×10⁶ cells/well and allowed to grow to 70% confluency in FGM™-2 medium. Self-amplifying RNA molecules are prepared as naked RNA in nuclease-free H₂O and then mixed together to provide reprogramming factor cocktails as follows: OSKMLN, OSKM, OSK, OSKG, or other combinations of the reprogramming factors, or cocktails with other combinations of reprogramming factors (see abbreviations above). The reprogramming factor cocktails contain the reprogramming factor-coding RNAs in identical proportions (e.g., 1:1:1:1:1:1 for O:S:K:L:M:N) or with proportions of individual factors adjusted (e.g., 2:1:1:1:1:1 for O:S:K:L:M:N). To provide an on-off switch, L7Ae-containing mRNA is used. RNA cocktails prepared in this manner are mixed with Lipofectamine™ MessengerMAX™ at a ratio of 1:1 to form transfection complexes per the manufacturer's instructions. The RNA transfection complexes are then added to the wells containing “aged” and “control” fibroblasts at doses of 0.1-20 ng/ml ng RNA per well, and transfection is allowed to proceed for 6 hours. Wells receiving Lipofectamine alone serve as a control. After transfection is complete, the transfection medium is discarded, and fresh medium is applied to the wells. Self-amplifying RNA is transfected once on Day 1, at the beginning of the experiment. L7Ae-containing mRNA is transfected to stop expression of self-amplifying RNA at 3, 4, 5, 6, or 7 days.

At 6, 7, 8, 9, 10, 11, 12, 13, or 14 days, cells are stained and analyzed by immunofluorescence to evaluate the expression of the reprograming factors; CD44, CD73 and CD105 as stemness markers; collagen 1A2, Heat shock protein 47 (HSP47), Fibroblast-specific protein 1 (FSP1), α-Smooth muscle actin (α-SMA), Serpin Family H Member 1 (SERPINH1), CD44, prolyl 4-hydroxylase (P4HB), S100 calcium binding protein A4 (S100A4), Thy-1 Cell Surface Antigen (THY1) as lineage-specific markers; collagen IV, fibronectin, and laminin as rejuvenation markers; and vimentin as an aging marker.

At 6, 7, 8, 9, 10, 11, 12, 13, or 14 days, cells are lysed, total RNA collected and reverse-transcribed to cDNA. Real-time PCR is used to evaluate the expression of the reprograming factors CD44, CD73 and CD105 as sternness markers; collagen 1A2, HSP47, FSP1, α-SMA, SERPINH1, CD44, P4HB, S100A4, THY1 as lineage-specific markers; collagen IV, fibronectin, and laminin as rejuvenation markers; and vimentin as an aging marker.

L7Ae on-off switch mechanisms shut off expression of the reprograming factors at the desired time point, reflected as decreased or undetectable expression of the reprogramming factors, whereas the reprogramming factor expression continue in cells treated with self-replicating RNA without an on-off switch. While all conditions show rejuvenation and de-aging, continued expression of the reprogramming factors by self-replicating RNA result in increased stemness and loss of cell identity and cell lineage. In contrast, by using the on-off switch to shut off expression of the reprogramming factors after rejuvenation and de-aging through epigenetic reprogramming occur, but before loss of cell identity and cell lineage, the stemness markers are not up-regulated, and cell identity and cell lineage markers are not down-regulated.

### Example 7

### Polycistronic RNA for Expression of Reprogramming Factors

Polycistronic RNA molecules encoding the reprogramming factors OCT4 (0), SOX2 (S), KLF4 (K), c-MYC (M), L1N28 (L), NANOG (N), and GLIS1 (G) (each molecule encoding two, three, four, five, or six factors, for example LMK and OSK) are synthesized via in vitro transcription from plasmid DNA and purified. Each mRNA molecule contains a 5′cap, 5′-UTR, coding sequences for two, three, four, five, or six factors, an IRES element or 2A element before each coding sequence such that each gene has its own IRES or 2A element, a 3′ UTR, and a polyA tail. Human fibroblasts are obtained from Lonza and cultured in FGM™-2 medium. An aging model is induced in the fibroblasts through treatment with TGF-beta at a concentration of 0.1-20 ng/ml for 3 days (“aged”). Untreated fibroblasts are used as a control (“control”). The model is described in detail in Juhl et al. (Scientific Reports volume 10, Article number: 17300 (2020)), incorporated herein by reference.

In other conditions, human fibroblasts from aged donors (for example, >65 years; “aged”) or young donors (for example, <25 years; “control”) are used. For example, neonatal human fibroblasts from newborn (“control”) and old human fibroblasts from 60+ year old (“aged”) are purchased from commercial manufacturer (Lonza). Gene and protein expression profiles are analyzed in “aged” cells that have been treated with reprogramming factors. Reprogramming factor treated “aged” cells exhibit gene and protein expression profiles skewed towards expression profiles seen in “control” cells. For instance, the expression profile of “aged” cells treated with reprogramming factors is shifted towards expression patterns that resemble the expression profiles of “control” cells when compared to the expression profiles of untreated “aged” cells.

For transfection, “aged” and/or “control” fibroblasts are seeded in 6-well plates at a density of 0.25×10⁶ cells/well and allowed to grow to 70% confluency in FGM™-2 medium. Polycistronic RNA molecules are prepared as naked RNA in nuclease-free H₂O and then mixed together to provide the full set of reprogramming factor combinations OSKMLN, OSKM, OSK, OSKG, or other combinations; for example, a polycistronic RNA encoding LMK could be mixed with polycistronic RNA encoding OSK. As a control, monocistronic mRNA, each encoding a single reprogramming factor, is used and mixed to provide OSKMLN, OSKM, OSK, OSKG, or other combinations. RNA cocktails prepared in this manner are mixed with Lipofectamine™ MessengerMAX™ at a ratio of 1:1 to form transfection complexes per the manufacturer's instructions. The RNA transfection complexes are then added to the wells containing “aged” and/or “control” fibroblasts at doses of 5000 ng RNA per well, and transfection is allowed to proceed for 6 hours. Wells receiving only vehicle (Lipofectamine™ MessengerMAX™) serve as a control. After transfection is complete, the transfection medium is discarded, and fresh medium is applied to the wells. The polycistronic RNA is transfected every day, every other day, every three days, every four days, or every five days, as is the monocistronic mRNA.

At 6, 7, 8, 9, 10, 11, 12, 13, or 14 days, cells are collected, and immunofluorescence used to evaluate expression of the reprogramming factors OCT4, SOX2, KLF4, c-MYC/GLIS1, LIN28, NANOG; CD44, CD73 and CD105 as sternness markers; collagen 1A2, HSP47, FSP1, α-SMA, SERPINH1, CD44, P4HB, S100A4, THY1 as lineage-specific markers; increased expression of collagen IV, fibronectin, and laminin as rejuvenation markers; and vimentin an as aging marker.

At 6, 7, 8, 9, 10, 11, 12, 13, or 14 days, cells are lysed, total RNA collected and reverse-transcribed to cDNA. Real-time PCR is used to evaluate the expression of the reprograming factors; CD44, CD73 and CD105 as sternness markers; collagen 1A2, HSP47, FSP1, α-SMA, SERPINH1, CD44, P4HB, S100A4, THY1 as lineage-specific markers; increased expression of collagen IV, fibronectin, and laminin as rejuvenation markers; and vimentin as an aging marker.

Compared to monocistronic RNA, use of polycistronic RNA increases the likelihood of all reprogramming factors, or the minimum amount of reprogramming factors required for effective epigenetic reprogramming to be present in the same cell, thus leading to higher reprogramming efficiency as determined by higher numbers of cells showing rejuvenation or de-aging, or higher expression of rejuvenation markers and lower expression of aging markers. Also, use of independent IRES elements for each reprogramming factor allows the relative expression ratio of the factors to be equal (e.g., 1:1:1:1:1:1 for O:S:K:M:L:N), or tuned/adjusted (e.g., 2:1:1:1:1:1 for O:S:K:M:L:N), thus increasing the reprogramming efficiency. Additionally, expression of the reprogramming factors from polycistronic RNA does not result in increased stemness or loss of cell identity or lineage.

### Example 8

### Circular RNA for Expression of Reprogramming Factors

Circular RNA molecules encoding the reprogramming factors OCT4 (O), SOX2 (S), KLF4 (K), c-MYC (M), LIN28 (L), NANOG (N), and GLIS1 (G) (each molecule encoding a single reprogramming factor) are synthesized via in vitro transcription from plasmid DNA, circularized, and purified. Each mRNA molecule contains a IRES element, the coding sequence for a single reprogramming factor, and a 3′ UTR.

Human fibroblasts are obtained from Lonza and cultured in FGM™-2 medium. An aging model is induced in the fibroblasts through treatment with TGF-beta at a concentration of 0.1-20 ng/ml for 3 days (“aged”). Untreated fibroblasts are used as a control (“control”). The model is described in detail in Juhl et al. (Scientific Reports volume 10, Article number: 17300 (2020)), incorporated herein by reference.

In other conditions, human fibroblasts from aged donors (for example, >65 years; “aged”) or young donors (for example, <25 years; “control”) are used. For example, neonatal human fibroblasts from newborn (“control”) and old human fibroblasts from 60+ year old (“aged”) are purchased from commercial manufacturer (Lonza). Gene and protein expression profiles are analyzed in “aged” cells that have been treated with reprogramming factors. Reprogramming factor treated “aged” cells exhibit gene and protein expression profiles skewed towards expression profiles seen in “control” cells. For instance, the expression profile of “aged” cells treated with reprogramming factors is shifted towards expression patterns that resemble the expression profiles of “control” cells when compared to the expression profiles of untreated “aged” cells.

For transfection, “aged” and “control” fibroblasts are seeded in 6-well plates at a density of 0.25×10⁶ cells/well and allowed to grow to 70% confluency in FGM™-2 medium. Circular RNA molecules are prepared as naked RNA in nuclease-free H₂O and then mixed together to provide the full set of reprogramming factor combinations OSKMLN, OSKM, OSK, OSKG, or other combinations. The reprogramming factor cocktails contain the reprogramming factor-coding RNAs in identical proportions (e.g., 1:1:1:1:1:1 for O:S:K:L:M:N) or with proportions of individual factors adjusted (e.g., 2:1:1:1:1:1 for O:S:K:L:M:N). As a control, linear mRNA molecules, each encoding a single reprogramming factor, are used, and mixed to provide OSKMLN, OSKM, OSK, or OSKG. RNA cocktails, which are mixed with Lipofectamine™ MessengerMAX™ at a ratio of 1:1 to form transfection complexes per the manufacturer's instructions. The RNA transfection complexes are then added to the wells containing “aged” and “control” fibroblasts at doses of 5000 ng RNA per well, and transfection is allowed to proceed for 6 hours. Wells receiving only Lipofectamine™ MessengerMAX™ serve as a control. After transfection is complete, the transfection medium is discarded, and fresh medium is applied to the wells. The circular RNA is transfected every day, every other day, every three days, every four days, or every five days, as is the linear mRNA.

At 6, 7, 8, 9, 10, 11, 12, 13, or 14 days, cells are lysed, total RNA collected and reverse-transcribed to cDNA. Real-time PCR is used to evaluate the expression of the reprograming factors; CD44, CD73 and CD105 as sternness markers; collagen 1A2, HSP47, vimentin, FSP1, α-SMA, SERPINH1, CD44, P4HB, S100A4, THY1 as lineage-specific markers; increased expression of collagen IV, fibronectin, and laminin as rejuvenation markers; and vimentin as an aging marker.

At 6, 7, 8, 9, 10, 11, 12, 13, or 14 days, cells are lysed, total RNA collected and reverse-transcribed to cDNA. Real-time PCR is used to evaluate the expression of the reprograming factors; CD44, CD73 and CD105 as sternness markers; collagen 1A2, HSP47, FSP1, α-SMA, SERPINH1, CD44, P4HB, S100A4, THY1 as lineage-specific markers; increased expression of collagen IV, fibronectin, and laminin as rejuvenation markers; and vimentin as an aging marker.

The use of circular RNA allows fewer transfections to be applied and lower RNA doses to be used as compared to conventional mRNA because of the persistence and lower immunogenicity of circular RNA. The need for fewer transfections and lower RNA doses also result in lower toxicity and higher reprogramming efficacy, leading to stronger cellular rejuvenation effects. Circular RNA also provides the benefit of higher cell viability and proliferation than conventional linear mRNA. Accordingly, when compared with linear mRNA, treatment with circular RNA results in upregulation of cell rejuvenation markers, and down regulation of cell immune response, toxicity, and aging markers.

While a number of exemplary aspects and embodiments have been discussed above, those of skill in the art will recognize certain modifications, permutations, additions, and sub-combinations thereof. It is therefore intended that the following appended claims and claims hereafter introduced are interpreted to include all such modifications, permutations, additions and sub-combinations as are within their true spirit and scope.

It is claimed:

1. A method of treating a cell, tissue, or organ in a subject in need thereof, comprising:
contacting the cell, tissue, or organ with a synthetic, persistent RNA vector comprising one or more heterologous polynucleotide sequences, each of the one or more heterologous polynucleotide sequences encoding for one or more reprogramming factors, whereby said contacting achieves expression of the one or more reprogramming factors in the cell, tissue or organ to obtain a rejuvenated cell tissue or organ with retention of cellular identity.

2. The method of claim 1, wherein the synthetic, persistent RNA vector is a self-replicating RNA vector that comprises a replicase domain.

3. The method of claim 2, wherein the replicase domain is a viral replicase domain.

4. The method of claim 3, wherein the viral replicase domain in an RNA viral replicase domain.

5. The method of claim 4, wherein the RNA viral replicase domain is from an RNA virus selected from the group consisting of an alphavirus, a flavivirus, a levivirus, a narnavirus, a mitovirus, a botourmiavirus, a picornavirus, a nidovirus, a sobelivirus, a potyvirus, and an astrovirus.

6. The method of claim 2, wherein the replicase domain is from:
(i) an alphavirus selected from the group consisting of Eastern Equine Encephalitis virus (EEE), Venezuelan Equine Encephalitis virus (VEE), Everglades virus, Mucambo virus, Pixuna virus, and Western Equine Encephalitis virus (WEE); or
(ii) an alphavirus is selected from the group consisting of Sindbis virus, Semliki Forest virus, Middelburg virus, Chikungunya virus, O'nyong-nyong virus, Ross River virus, Barmah Forest virus, Getah virus, Sagiyama virus, Bebaru virus, Mayaro virus, Una virus, Aura virus, Whataroa virus, Babanki virus, Kyzylagach virus, Highlands J virus, Fort Morgan virus, Ndumu virus and Buggy C.

7. The method of claim 2, wherein the self-replicating RNA vector comprises from 5′ to 3′: (a replicase domain from an RNA virus)-(a promoter)-(a first reprogramming factor)-(a first reprogramming factor separating region)-(a second reprogramming factor)-(a second reprogramming factor separating region)-(optional additional reprogramming factors-optional additional separating regions)-(optional selectable marker)-(virus 3′UTR or a polyA tail)-(optional selectable marker)-(optional promoter).

8. The method of claim 7, wherein the reprogramming factor separating regions each consist of one or more of an IRES, a promoter and a self-cleaving peptide, wherein the IRES is derived from a virus, a bacterium, or a eukaryote, or is synthetic in origin.

9. The method of claim 1, wherein the synthetic, persistent RNA vector is a circular polyribonucleotide.

10. The method of claim 9, wherein the circular polyribonucleotide comprises one or more polynucleotides encoding for a reprogramming factor.

11. The method of claim 10, wherein the circular polyribonucleotide further comprises one or more of an encryptogen, a regulatory element and a replication element.

12. The method of claim 9, wherein the circular polyribonucleotide provides expression of the one or more polynucleotides encoding for a reprogramming factor at a level that does not vary by more than about 40% for at least about 2, 3, 4, 5, 6, 7, 8, 9, 10, 12, 14, or 16 days.

13. The method of claim 1, wherein the reprogramming factor is selected from the group consisting of Oct polypeptides, Klf polypeptides, Sox polypeptides, Myc polypeptides, Nanog, Lin28, and Glis.

14. The method of claim 1, wherein the reprogramming factor is selected from:
(i) the group consisting of OCT3, OCT4, SOX2, KLF4, c-Myc, and Glis1; or
(ii) the group consisting of OCT4, SOX2, KLF4, c-Myc, and Glis1.

15. The method of claim 1, wherein the synthetic, persistent RNA vector, comprises one or more heterologous polynucleotide sequences encoding for one or more reprogramming factors, wherein the one or more heterologous polynucleotide sequences have at least 95% sequence identity to any one of SEQ ID NOs: 1-6 and 10.

16. The method of claim 1, wherein said method is for treating a differentiated cell or an age-related disease or condition.

17. The method of claim 1, wherein said contacting is in vitro or ex vivo and the method further comprises transplanting the rejuvenated cell into a subject.

18. The method of claim 1, wherein said contacting is in vivo and achieves transfection of the mRNA encoding one or more reprogramming factors into the cell for expression of the one or more reprogramming factors intracellularly.

19. The method of claim 1, wherein the cell, tissue or organ is a somatic cell from a human subject.

20. The method of claim 1, where the cell is associated with a tissue or organ and the tissue or organ is skin, hair, lung, cartilage, or eye.

21. The method of claim 1, wherein said contacting comprises contacting the cell to the self-replicating RNA once, and the self-replicating RNA is capable of expressing the one or more reprogramming factors for a period sufficient for therapy.

22. The method of claim 21, wherein the period sufficient for therapy is:
(i) for 1 to 30 days; or
(ii) up to 12 weeks; or
(iii) up to 24 weeks; or
(iii) up to 52 weeks.

23. The method of claim 16, wherein the age-related disease or condition is a dermatologic disease or condition, an eye disease or condition, a respiratory disease or condition, a musculoskeletal disease or condition or a cellular proliferation disorder.

24. The method of claim 23, wherein:
the dermatologic disease or condition is dermal atrophy, dermal elastolysis, skin wrinkling, sebaceous gland hyperplasia, sebaceous gland hypoplasia, senile lentigo, a pigmentation abnormality, graying hair, hair loss, hair thinking or a chronic skin ulcer; or
the eye disease or condition is age-related macular degeneration, glaucoma, a cataract, dry eye, diabetic retinopathy, or vision; or
the respiratory disease or condition is pulmonary fibrosis, chronic obstructive pulmonary disease, asthma, chronic bronchitis, pulmonary embolism, lung cancer or a lung infection; or
the musculoskeletal disease or condition is arthritis, osteoporosis, myeloma, gout, Paget's disease, bone fracture, bone marrow failure syndrome, ankyloses, diffuse idiopathic skeletal hyperostosis, hematogenous osteomyelitis, muscle atrophy, peripheral neuropathy, multiple sclerosis, amyotrophic lateral sclerosis, Duchene muscular dystrophy, primary lateral sclerosis, or myasthenia gravis; or
the cellular proliferation disorder is a cancer.


SEQUENCE LISTING (19 sequences)

SEQ ID NO: 1 - DNA, 1083 bp, synthetic construct
  source 1..1083: mol_type = other DNA
atggctggccatctcgcaagtgacttcgcattttccccgcccccaggcggcggtggagatggacctggcggcccagaaccaggatgggtggacccccgcacgtggctttcttttcagggtccaccaggcggacccggtattggacccggcgtgggtcctgggtcagaagtctggggtatcccaccctgtcccccaccatacgaattttgcggcggaatggcctattgcggccctcaagtcggggtcggtctggtacctcagggtggtctggaaacatcccaaccagaaggtgaggccggtgtgggagttgaatccaattctgacggagcatctccagaaccttgtactgtgacaccaggagctgttaaattggagaaagaaaagctcgaacagaatccagaagaatcacaagatattaaggcgctccaaaaggagctggaacagttcgcgaaacttcttaaacagaaacgcattactctcgggtacacccaagcggacgttggactgactctcggtgtgctgttcggcaaagtctttagtcagaccacaatatgtcgattcgaagcccttcaactgtcatttaagaatatgtgcaaacttcgacctctgctccagaaatgggtcgaagaggcggataataacgagaacctgcaagaaatctgtaaggcggagactctggttcaagctcgcaaaaggaaacgtacgtctatagaaaatagagtccgtgggaatcttgaaaacctgtttctccaatgtccaaagcctactttgcaacaaatatctcatattgcgcaacaactcggcctggaaaaggacgtagttagagtctggttttgcaatcgcagacagaaagggaaacggtcttccagtgattacgcgcagagggaagacttcgaagcagccggttcaccgttttccggcggcccggtatctttcccattggctcccggtcctcacttcggcacacccgggtacggctcaccacattttaccgccctttattcaagcgttccctttccggaaggcgaggctttcccgccggtgtcagtgactacacttggatccccaatgcacagcaattag

SEQ ID NO: 2 - DNA, 954 bp, synthetic construct
  source 1..954: mol_type = other DNA
atgtataatatgatggaaaccgaattgaaaccacccgggccccaacagacctctggcggcggtggtggtaatagcacagcagcagccgctggtggaaatcaaaagaattctccagatagagtgaaacgacctatgaacgcatttatggtctggtctagaggacaacgaaggaaaatggctcaagaaaatcccaaaatgcataatagcgaaatttccaaacggttgggtgcggaatggaagctcctcagcgaaaccgaaaagaggccatttattgatgaagcgaaaagactcagggcattgcatatgaaagaacatccagactacaagtatagaccacgccgcaagacaaagactctgatgaagaaggacaaatataccctgcctggtggattgttggctcctggcggtaacagtatggcttctggcgtgggcgttggggctggacttggtgccggggtcaatcaacgaatggattcctatgcccatatgaatggatggagtaatggttcctattctatgatgcaagatcaattgggatatcctcaacatcccggtctgaacgctcatggtgctgctcaaatgcaacctatgcatcggtatgatgtaagtgcattgcaatataatagcatgacatccagtcaaacatatatgaatgggtcaccaacatatagtatgagctattcccaacaaggtacaccagggatggccctggggagcatggggagtgtcgttaaaagtgaagcttcaagttcaccacccgtagtgacgagttcttcacattctcgagccccatgtcaagcaggagatcttagggatatgatttcaatgtacttgccaggggctgaagtccccgagccggcagcgccttctaggctgcatatgtctcaacattatcaatccggacccgttccagggaccgctatcaatggtacgttgccattgtcccatatgtaa

SEQ ID NO: 3 - DNA, 1365 bp, synthetic construct
  source 1..1365: mol_type = other DNA
atggacttctttcgagtggtagagaatcaacaacccccggccaccatgccattgaatgtatcatttacaaaccggaactacgacctggactatgattcagttcagccttacttttattgtgacgaagaagaaaatttctatcaacaacaacaacaatcagaattgcaaccacccgctccatcagaagacatttggaagaagtttgaactcctgccaactccaccgctcagcccgagtagacgttctggactgtgttctccttcttatgtggctgtgactccgttttcactgcgtggcgataatgatggcggcggtggctcttttagtacagcagatcaacttgaaatggtcacagaactccttggtggtgatatggttaatcaatcattcatttgtgatcccgatgatgagacatttataaagaacatcatcatacaagactgcatgtggtcagggtttagtgctgctgcgaaactggtgagcgaaaagttggcttcttatcaagccgcccggaaggatagtggatcaccaaatccagcaaggggtcattcagtgtgtagcacaagctctctgtatcttcaagacctctccgcggctgcaagtgaatgtattgatccaagtgtcgttttcccttatcccctgaatgattcttcctctcctaaaagctgtgcgagccaggattcttcagctttctccccaagctccgacagtttgttgagctctactgaaagtagtcctcaagggtcaccggaacctctcgtccttcacgaagaaacaccccctacaactagttccgattccgaagaagaacaggaagacgaagaggagattgacgtggtatcagttgagaaaagacaagcccccgggaagcgaagcgaaagcgggagcccaagcgccggcggacattccaagcccccacattctcctttggtactgaaaagatgtcatgtgagcacccaccaacataattatgctgctcccccatcaaccaggaaagattaccccgccgctaaacgagttaaactggattcagtgagggttcttaggcaaatttcaaataataggaagtgtacttcacctcgcagtagcgatacagaagaaaacgttaaaagacggacgcataatgtgctggaacgacaaagacgaaatgaacttaagaggtccttcttcgcgcttagggatcaaatacctgaactggagaataacgagaaagctccaaaagtggtgattttgaagaaggctactgcgtatatactttctgtacaggccgaagaacagaaactgatatcagaggaagatctgcttcgtaagcgcagagagcaactgaagcataagctcgagcaactccgcaatagctgcgcctag

SEQ ID NO: 4 - DNA, 1542 bp, synthetic construct
  source 1..1542: mol_type = other DNA
atgcggcagccaccaggggaaagtgatatggccgtttccgacgctcttctgccttcattttcaacctttgcttccggacctgccgggcgggaaaagacgcttaggcaggccggagcacccaacaatcgatggagagaagaactgagccatatgaaaagactgccgcctgtactcccggggcggccatacgatctcgccgccgctacagtagcaactgatttggaatccggtggtgctggggcagcctgtggcggatctaatcttgctcctctgccaagacgggaaacggaagaatttaatgacctgctcgatttggatttcatcctttctaactcacttacacacccaccagaaagtgtcgctgctacggtctctagctctgccagcgcaagcagttcatcttctccatcttcctcaggacccgcaagtgccccgagcacttgttcctttacgtacccaatacgagctggcaatgatcctggagttgctcccggtggtacaggcggtggtctgttgtacggaagagaatcagccccaccaccaaccgcaccatttaatttggccgatattaatgatgtttcacctagtggtggttttgttgcggaactgctccgtcccgagctggatcccgtctatatcccaccacaacaaccccaaccacctggcggcggattgatgggtaaatttgttcttaaagcatccctttccgcaccagggtcagaatatggaagtccctcagtgatttccgtatctaagggatcccccgatgggtcccatccagttgtcgttgccccttataatggtggcccgcctagaacctgtccgaaaataaaacaagaagctgtatcatcatgtacgcatctcggggcaggtccacccctgtctaacgggcatcgccctgcagcccatgattttcctttgggccgacaactgccgtcccgtacaactccaacactcggcctcgaagaggtcctcagtagtagagattgccatccagcactccccctgccaccaggttttcaccctcatcccggtcctaactatcccagctttctcccagaccaaatgcaaccccaggttcccccacttcactatcagggccaaagcagaggtttcgtggcgcgagccggagaaccatgcgtctgttggcctcattttggcacccatggaatgatgttgacacccccaagtagtccactcgaactgatgccccctggcagttgtatgcctgaagaacctaaacccaaaagagggcggcggagttggccacgaaagcgaacagcaactcatacctgcgactatgctggatgtgggaagacatataccaaatctagccacctgaaagcgcatctccgcacgcatactggagaaaagccctatcattgcgattgggatgggtgcggctggaagtttgcacgatctgacgagcttactagacattatcgcaagcataccggacatcggccctttcaatgtcagaagtgtgatcgcgccttcagccggagcgatcatctggctctgcatatgaaacgacacttctag

SEQ ID NO: 5 - DNA, 630 bp, synthetic construct
  source 1..630: mol_type = other DNA
atgggctcagtcagcaatcaacaattcgcgggtggatgtgcaaaagctgctgaggaagccccagaagaagctccagaagatgccgctcgcgccgctgatgaaccacaactgctccatggagctgggatttgcaaatggtttaatgtccggatgggctttggtttcttgtctatgacagcaagagctggagtggccctcgatccacccgtcgacgtgttcgtacatcaatctaaacttcatatggaaggatttcgctcactcaaagaaggagaagccgtagaatttacattcaagaaaagtgcgaaagggcttgagagcatacgggtgacagggccaggcggcgtgttttgcatcggatcagaacgaaggcccaagggtaaatccatgcaaaagaggcgatccaaaggggatcggtgttataattgcggtggccttgaccaccacgcgaaagagtgtaaacttcccccgcaacctaagaaatgtcatttctgtcaatccatatcacacatggtggcgagctgcccattgaaagcacaacaaggaccgtccgcccaagggaaacctacttatttcagggaagaggaagaggagattcatagtcccactctccttcctgaagcccaaaactag

SEQ ID NO: 6 - DNA, 918 bp, synthetic construct
  source 1..918: mol_type = other DNA
atgtcagtcgacccggcctgcccacagagtctgccctgtttcgaggcttcagattgcaaggagtcctctccgatgcccgtcatctgcggacccgaggagaattaccccagtctgcagatgtccagtgccgaaatgccacatacagaaacggtttcaccgctcccatcttcaatggaccttttgatccaagattctcccgacagcagcacttccccaaagggaaagcagcctacctcagcggagaaatctgtggccaagaaagaggataaagttcctgttaagaagcaaaagacaaggaccgtattttcctcaacacaactctgcgtccttaacgaccggttccaacgccaaaagtatctgtccttgcaacaaatgcaggagctgagcaatattttgaatctgtcttataagcaagtcaagacttggtttcaaaaccagcgtatgaagagtaaaagatggcagaagaataattggcccaagaactctaacggcgttactcaaaaggcgagtgcccccacttatccatctctgtattcatcatatcatcaaggctgtttggttaatcccacgggcaatctgccgatgtggtccaatcaaacgtggaataacagtacttggtccaatcaaactcaaaatatacaaagttggtcaaatcattcttggaatacccaaacgtggtgtactcagagctggaataaccaagcatggaactccccattttacaattgcggcgaagagtcactccaaagctgtatgcaatttcaacccaacagtcccgcatccgatcttgaagcagcactggaagcggccggtgaggggctgaacgtgattcaacaaacaacaagatacttctccacacctcagactatggaccttttcctgaattattcaatgaatatgcagcccgaggatgtctag

SEQ ID NO: 7 - DNA, 115 bp, synthetic construct
  source 1..115: mol_type = other DNA
aaaaaaaaaaaaaaaaaaaaaaaaaaaaaaaaaaaaaaaaaaaaaaaaaaaagcatagcagctgactaaaaaaaaaaaaaaaaaaaaaaaaaaaaaaaaaaaaaaaaaaaaaaaa

SEQ ID NO: 8 - DNA, 87 bp, synthetic construct
  source 1..87: mol_type = other DNA
ttggaccctcgtacagaagctaatacgactcactatagggaaataagagagaaaagaagagtaagaagaaatataagagccaccatg

SEQ ID NO: 9 - DNA, 119 bp, synthetic construct
  source 1..119: mol_type = other DNA
gctgccttctgcggggcttgccttctggccatgcccttcttctctcccttgcacctgtacctcttggtctttgaataaagcctgagtaggaagtgagggtctagaactagtgtcgacgc

SEQ ID NO: 10 - DNA, 1863 bp, synthetic construct
  source 1..1863: mol_type = other DNA
atggccgaagccagaacatctctgtctgcccactgtagaggccccctggctacaggtctgcaccccgacctggatctgcctggccggagcttggccacccctgctcctagctgctacctgctgggctctgaacccagctccggccttggcctgcagcctgagacacacctgcctgaaggctccctcaagcggtgctgcgtgctgggcctgcctccaacatcccctgcttcctcttctccatgcgccagcagcgacgtgaccagcatcatcagaagcagccagacaagcctcgtgacctgcgtgaacggcctgcggagccctcccctgaccggcgatctgggaggcccctcaaagagagcccggcccggccctgccagcaccgacagccacgagggcagcctgcagctggaagcttgtagaaaggccagcttcctgaaacaggagcctgccgacgagttctccgagctgttcggcccccaccagcagggactgccgcctccttatcccctgtcccagctgcccccaggaccaagtctgggaggactcggcctgggactggccggcagagtagtggccggaagacaggcctgccggtgggtggactgttgtgccgcttacgagcagcaggaggaactggtgcgccacatcgagaaaagccacatcgaccagaggaagggcgaggatttcacatgcttctgggccggctgcgtgcggcggtacaagccctttaatgccagatacaagctgctgatccacatgagagtgcacagcggcgaaaagcctaacaagtgcatgttcgagggctgcagcaaggccttctctagactggaaaatctgaaaatccacctgagatctcacaccggagaaaagccttacctgtgccagcatcctggatgtcagaaggccttcagcaacagctctgatagagccaagcaccagagaacccacctggacaccaagccttatgcctgccagatccctggctgcagcaaaagatacaccgatccttctagcctgcggaagcacgtgaaggcccatagcgctaaggaacagcaggtgcgcaaaaagctgcatgccggcccagataccgaggccgatgtgctaactgagtgcctggtcctgcaacagctgcacacatctacccagctggccgccagcgacggcaagggcggatgtggcctgggacaggagctgctgcctggtgtttaccccggaagcatcacccctcacaacggcctggccagcggactgctgccccccgcacacgacgtgcctagccggcatcaccctctggacgccaccaccagcagccaccaccacctgagccccctgcctatggccgaaagcaccagagatggcctcggccctggcctcctcagccctatcgtgtctccactgaaaggcctgggcccccctcctcttcctccaagcagccagagccacagccccggcggccagcctttccctaccctgccttctaagcccagctaccctccttttcagagccctcctccccctccactgccctctcctcagggctaccaaggcagcttccactctatccagtcctgttttccttacggcgactgctacagaatggccgaaccagccgccggcggcgacggcctggtgggcgagacacacggctttaaccccctgagacctaacggctaccactctctgagcacacctctgcctgctaccggctacgaggctctggcagaggccagctgccctaccgccctgccgcaacaacctagcgaagacgtcgtgtcctctggcccagaggactgcggattcttccccaacggcgccttcgaccactgcctgggccacattcctagcatctacaccgacacatga

SEQ ID NO: 11 - DNA, 1281 bp, synthetic construct
  source 1..1281: mol_type = other DNA
atggagttcgccatggagctgctgtcgccaccgttgcgtgacgtggacctgacagcccccgacggctctctgtgctcctttgctactaccgacgatttctacgatgacccgtgctttgattctcccgacctgcgctttttcgaagacttagatccgcgcctgatgcatgtaggtgctctgctaaagcccgaggagcacatggctggccacttggcttccgacttcgcgttctccccgccacccggcggcggaggcgatggccctggcggacccgagccaggttgggtcgaccctcggacctggctctccttccagggccccccaggcgggcccggcataggtcccggcgttggacccgggagcgaggtgtggggcatccctccgtgcccacccccgtatgagttctgcgggggtatggcctactgcgggcctcaggtgggcgtcggcctcgtccctcaggggggtctggagacctctcagccggagggagaggctggggtcggcgtggagagcaactccgacggggcgtcgcccgaaccttgcactgtcacgcccggggccgttaagctggagaaggagaaacttgagcagaaccccgaggagagccaggacatcaaggcgctgcagaaggagctggaacagttcgccaagctgctgaagcagaagcgcatcaccctaggttacacccaggcggacgtgggcctgacgcttggtgtgctgttcggaaaggtgttcagccagacgaccatctgccgattcgaggccctccagctgtccttcaagaacatgtgcaagttgcggcccctgctccaaaaatgggtggaggaggctgacaacaacgagaatctccaggagatctgtaaagccgagactctggtgcaggcccgcaaacgcaagcgtacctcgattgaaaacagggtgcgtggcaacctggagaacctgttcctacagtgtcccaagcctacccttcagcagattagccacatcgcacaacagttgggccttgaaaaggatgtggtacgcgtgtggttctgtaaccgccgccagaagggtaagcgcagctccagcgactacgcgcagagagaggactttgaggctgcaggatctcctttttctggcggccctgtgagtttccctctggccccgggaccccactttggtactccgggctacggctccccgcacttcaccgccctgtactctagtgtcccgtttcccgagggcgaggcgttccccccagtgtccgtgaccacactggggtccccaatgcattcaaattga

SEQ ID NO: 12 - DNA, 1056 bp, synthetic construct
  source 1..1056: mol_type = other DNA
atgacaatgaaaatgatggtgcacatctacttcgtctctcttctgctgctgctgtttcacagctacgcaattgacatcgaaaacgagatcaccgagttcttcaacaagatgcgtgacacccttcccgccaaggattctaaatggctcaacccggcctgcatgtttggaggcaccatgaacgacatcgcggcgctgggcgagcccttctccgctaaatgtcccccgatcgaagattctctgctgagccaccgctacaaggactatgtggtcaaatgggagcgcctggagaagaaccgtcggcgccaggtctcaaacaagcgcgtaaagcatggagatctttggatcgccaactacaccagtaaatttagcaaccgccgctacctgtgcactgtcaccaccaagaatggggactgcgtgcagggcatcgtgcggagccacatccgcaagcctccctcttgtattcccaagacctacgagctggggacacatgacaagtacggcattgacctgtattgcgggatcctgtacgcgaagcactacaacaacatcacctggtacaaggacaacaaggagattaacatcgatgacatcaagtactcccagactggcaaggagctcatcatccacaaccctgagctggaggactccggtagatatgattgttacgtgcattacgacgatgtgcgcatcaaaaacgacattgtcgtttccaggtgtaagatactcactgtgatccctagtcaggaccaccgcttcaagctgatcttggacccgaagataaatgtgaccatcggggagccagccaatatcacctgcactgccgtgtccacctcgttgctcattgacgatgtactgattgagtgggagaatccatcgggttggctaatcggcttcgacttcgacgtgtactcggtgctaacctcccgtggtggcatcactgaggctacgctttatttcgagaacgtgacggaggagtacatcggcaacacctacaagtgccgaggccacaactactacttcgaaaaaaccctgacgacgaccgtggttttggagtga

SEQ ID NO: 13 - DNA, 1542 bp, synthetic construct
  source 1..1542: mol_type = other DNA
atgcgtcagccacccggggagagcgacatggccgtgtcggacgcgctgctgccatccttttccaccttcgcctcgggtccggccggccgagagaagactctgcgccaggccggagcccctaacaaccgctggagagaggagctgtcacacatgaaacgcctgccccccgtgctgcctgggcgcccctacgaccttgccgcggccacggtggctaccgacttggagtctggaggtgctggagcagcgtgtggcggaagcaacctggcaccgttgccacgccgggagaccgaggagttcaacgacttgttggatctggactttattctgtccaactcccttacacacccgcccgagagcgtagcagccaccgtgagctccagtgcttccgcttcctcctcatccagcccgtcgtcttctggccctgcctctgcgccgtcgacctgttcgttcacctatcccatccgggccggcaacgatccgggcgtggccccgggcggcaccggtggtggtctcctgtacggcagggagtccgccccccctccaaccgctcccttcaacctcgcggacatcaatgacgtgtccccctctggcggcttcgttgcagaactgttaaggcctgaactggatccagtgtacatcccgccccagcagcctcagccgccgggcggcggtctgatgggcaaatttgtcctgaaggcgtctctgtctgctcctggctccgagtacggcagccccagtgtgattagcgtgtctaaaggcagccccgacgggtcgcaccccgtggtggtcgctccttacaacggtggacccccgcgcacctgcccaaagatcaagcaggaggctgtttcttcatgcactcatctaggcgccggtccacccctttccaatggccaccggcccgccgcgcatgacttccccctgggccgccagctgcccagccggaccacacctaccttaggcctggaggaggtgcttagttcgcgcgactgtcatcctgccctgcctctccctccaggcttccacccccacccgggacccaactacccgtccttcctgcccgaccagatgcaacctcaggtccctcccctgcactaccagggacagagccgcggtttcgtcgcccgtgctggcgagccatgcgtctgttggccgcacttcggcacgcatggaatgatgctaactcccccgagctcccccctggagctgatgcccccgggttcctgtatgccagaggagcccaagcctaagcgcggcagacgtagttggccccgtaagcgcaccgccacacatacgtgcgactacgcggggtgcgggaaaacctacaccaagtcttctcacctcaaggctcacttgcgtactcacactggagagaagccttatcactgcgactgggatgggtgcgggtggaagtttgctcgctccgacgaactcacccgccattaccgcaagcacaccggccaccgccccttccagtgtcagaagtgcgatcgagcgttctcccgctcggaccacctggccctacacatgaagaggcacttttga

SEQ ID NO: 14 - DNA, 630 bp, synthetic construct
  source 1..630: mol_type = other DNA
atgggctccgtgtcgaatcagcagttcgcaggcgggtgcgccaaggcagccgaggaggccccggaggaggctcctgaagacgccgctcgcgcggcggacgagcctcagctactccatggagctggcatctgcaaatggttcaacgtgcgaatgggatttggcttcctgtctatgactgctcgtgccggcgtggcgctggacccgcccgtagacgtgttcgtccaccagagcaagctgcatatggagggtttcagatctctgaaggagggcgaggccgtggagttcacgttcaagaagtcggccaaaggtctggagagcatccgcgtcaccggtcccggcggcgttttttgtattggctccgagcgccggcccaagggcaagtccatgcagaagcgccgctccaagggggacaggtgttacaactgcgggggtttggatcaccacgcgaaggagtgcaaacttcctcctcagccgaagaaatgtcacttttgtcagagcatcagtcacatggtggcttcatgcccactcaaggcccaacagggaccctctgcgcagggcaagcccacctacttccgcgaggaggaagaagagatccactccccaaccctgctgcccgaggctcagaactga

SEQ ID NO: 15 - DNA, 918 bp, synthetic construct
  source 1..918: mol_type = other DNA
atgtctgttgacccggcctgcccacagagccttccctgctttgaggcttccgactgtaaagagagttccccgatgcccgtgatttgtggtcctgaagagaactacccgtccctacagatgtcatcggcggagatgcctcataccgagaccgtgtcccccttaccctcttctatggatctgctgatccaggactcgcccgacagctcgaccagccctaagggcaagcagccaacctccgcagagaagtccgtggctaagaaggaggacaaagtaccggtgaagaaacagaagactcgcaccgtgttctcgtcgacccagctgtgcgtgctcaacgacaggtttcagcgccagaagtacctgtccctgcagcagatgcaagagctcagcaacatcctgaacctgagctacaagcaggtcaagacctggttccagaaccagcgtatgaagtccaagcggtggcaaaaaaacaactggcccaagaacagcaacggtgtcacccagaaggcctccgcccccacctacccgtctctttactccagctatcaccagggctgcctggtgaatccaacaggaaacctgcccatgtggtctaatcagacctggaacaactccacctggtctaaccagacacagaacatccagagttggtctaatcactcttggaacacgcagacctggtgcactcagagttggaataaccaggcctggaattcccccttctacaactgcggggaggagagcttgcagtcatgtatgcagttccagcccaactccccagccagtgacctggaggccgcgctggaggcggctggcgagggcctcaacgtgatccaacagacgacccgctacttctccacgcctcagactatggatttgttcctgaactattcgatgaacatgcagcctgaagatgtgtga

SEQ ID NO: 16 - DNA, 1083 bp, synthetic construct
  source 1..1083: mol_type = other DNA
atggcgggccacttggcttccgacttcgcgttctctcccccgcctggcggaggcggggacggccccggcggccctgagccaggctgggtcgatccacgcacctggctctccttccagggaccccccggggggcccggcatcggtcccggggtaggccccggatcggaggtgtggggcatcccgccgtgcccaccgccctatgagttctgtgggggtatggcctactgcgggcctcaggtcggtgtcggtctggtgcctcagggcggtctcgagaccagccagccggagggcgaggctggcgtgggtgtggagagcaacagcgatggcgcttctcctgaaccatgcactgtcacccccggcgccgtgaagctggagaaagagaaactggagcagaatccagaggagagtcaggacatcaaggccctgcagaaggaactggaacagttcgccaagctgctgaagcagaagcgtatcacccttggatacacccaggcggacgtgggcctcactttaggcgttcttttcggaaaggtgttctcacagaccacaatctgccgattcgaggccctgcaactttctttcaaaaacatgtgcaagttgcggcccctactgcagaaatgggtggaggaagcggacaacaacgagaatctgcaggagatctgtaaggccgagacactggtgcaggctcgcaagcgcaaaagaacgagcattgagaaccgcgtccgcggcaacctggagaacctgttcctgcagtgcccaaagcctaccctccagcaaattagccacatcgctcagcagctgggcctggagaaggatgtggtgagggtgtggttctgtaaccggcgccagaagggcaagcgcagctccagtgactacgcacagcgtgaggactttgaagccgccggctccccgttttcaggaggccccgtgtcctttcctttggctcccggccctcatttcggtactccgggctacggctccccccactttaccgccctttactcctctgtccccttccccgagggagaggcatttcccccggtgtccgtaacgaccctgggttccccaatgcactctaactga

SEQ ID NO: 17 - DNA, 954 bp, synthetic construct
  source 1..954: mol_type = other DNA
atgtataacatgatggaaacagagctgaagcccccggggcctcaacagacctccggcggtgggggcggcaactcgaccgctgccgcagctggtggaaaccagaagaacagtcccgacagagttaagcgcccgatgaacgcgttcatggtgtggtctcgcggccagcgccgcaagatggcgcaggagaatccaaaaatgcacaactcggagatctccaagcggctcggtgccgagtggaagctgctaagcgagaccgagaaacgtccttttattgacgaggccaagcgcctgcgtgcgcttcacatgaaggagcaccccgactacaagtacaggccccgacgcaaaaccaagaccctgatgaaaaaggacaagtacaccctccccggcggcctgctggcccctggtggcaacagcatggcctccggagtcggggtaggcgccggccttggagctggagtcaaccagcgtatggattcttacgcgcacatgaatgggtggtcaaatggctcgtattctatgatgcaggaccagctgggctaccctcaacaccccggcctcaacgcccatggagcggctcagatgcagccaatgcaccgctacgatgtgagcgccctgcagtacaactctatgactagttcacagacttacatgaacggttccccaacctactccatgtcttacagtcagcagggaacgccgggtatggctctgggctccatgggctccgtggtgaagtcggaggcatcctccagccctcccgtggtcacctcctcctctcacagccgcgctccttgccaggccggggacctgcgcgacatgatctctatgtatctgcccggtgcagaggtgcctgaaccggcggccccctctcggttgcatatgtcccagcattaccagagcggcccggtgccaggcactgccatcaacggcaccttgcccctgagccacatgtga

SEQ ID NO: 18 - DNA, 1365 bp, synthetic construct
  source 1..1365: mol_type = other DNA
atggatttcttccgagtggtggagaatcagcagccgcctgccaccatgccccttaacgtgtccttcactaacagaaactacgacctggactacgacagtgtccagccctatttctactgtgatgaggaggagaacttttaccagcagcaacagcagagcgaactgcagcccccagcgccctcggaggacatctggaagaaatttgagctgctgccaaccccccccctatctccatcccgccgttccggtctctgctctcctagctacgtggctgtcactcccttttcgctgcgtggggacaacgatggggggggcggaagtttcagcactgcggaccagctggagatggtgaccgagctgctgggtggtgacatggtcaaccagtcttttatctgcgacccggatgacgagaccttcatcaagaacatcatcatccaggactgtatgtggtctggcttctctgccgctgcaaaactggtctcggagaaacttgctagctaccaggctgctcgcaaggactccggctcgccgaatccagctaggggacatagtgtttgtagtacctcctcgctctacctgcaggacctgtccgcagcggcctctgagtgtattgacccgtccgtggtgttcccctatcctctcaacgactcgtcttcccccaagagctgcgcctcccaggactcatctgcgttctccccctcctccgatagcctgttgagtagcacagagagctcccctcagggctccccggagcccctggtgctacacgaggagaccccgcccaccaccagcagtgactcagaagaggagcaggaggacgaagaggagatcgatgtggtttccgtggagaagcgccaggcccctggcaaacgctccgaatccggctccccttccgccggcggccactccaagcccccgcacagcccgttggtgctgaagagatgtcacgtgtcaacccaccagcataactacgcggcgcctccctctacccgcaaagactaccccgctgccaagcgcgtgaagttggatagcgtccgggtcttacgccagatttctaacaaccgcaagtgcacttctccaaggtcttccgacacggaggagaacgtgaagcggcgcacccacaacgtactggagcgccagcgtcgtaacgagttgaagcgcagcttcttcgcgcttcgggaccagatacctgagctcgagaataacgagaaagcaccaaaggtagtcatcctgaagaaggccacggcctacatcttatcagtgcaggccgaggagcagaagctgatttcagaggaggatctgctgcgcaagcgccgagaacagctgaagcacaagctggaacaactccgcaactcatgcgcctga

SEQ ID NO: 19 - DNA, 1863 bp, synthetic construct
  source 1..1863: mol_type = other DNA
atggccgaggcccgcactagcctctcagcgcactgtcgcggccctctcgctacgggcctgcaccccgacctggacctgcccgggcgcagcctggctaccccggccccctcctgttaccttttgggctcggagccctctagcggcctgggcctacagccagagactcaccttcccgaaggctccctgaagaggtgttgcgtccttggcttgccgcccacctcgcctgccagctcctccccatgcgcgtcttcagacgtgacctccatcatccgctcgtcccagacctccctggtgacctgcgtgaacgggctgcgttctcccccgctcaccggcgacctgggaggtccgagtaagcgcgctcggcccggccctgcgagtacagactctcatgagggctcgctgcagctggaagcgtgtcgcaaggcctccttcctgaagcaggagccagccgacgagttctctgagctgttcggacctcaccagcagggtttgccccctccttacccactcagccagctcccccctgggccctctttgggcggtttaggcctggggttagcgggacgtgtggtggctggccgccaggcctgccgatgggtcgattgttgtgccgcttacgagcagcaggaggagctggtgcgccacattgaaaagagccatattgaccagcgcaagggcgaggacttcacctgcttttgggctgggtgcgtgcgccgctacaagccgttcaacgcgagatacaagctgctgatccacatgcgtgtacactccggagagaaacccaacaagtgcatgtttgagggatgctccaaggcgttcagccgcctggagaacttgaaaatccacctgcgctcacacaccggtgagaagccatacctgtgccaacaccccggttgtcagaaggcgttcagcaacagctccgatcgagctaaacaccagcggacccatcttgacaccaagccgtacgcatgccaaattccagggtgctccaagcgttacaccgacccgtccagtctgcgcaagcacgtgaaggcccatagtgcgaaggagcagcaagtgcggaagaagctccacgccggccctgacacggaggccgatgtgctgaccgagtgcctggttctgcagcagctgcacactagcacccagctggcagccagcgatggcaaaggtggttgcgggctgggacaggagttactgccaggcgtgtaccccggaagcatcaccccgcacaacggcctagcctctggacttctccctcctgcccatgacgtgccctctcggcaccacccgctcgatgctaccacctcctctcaccaccacctgtcccccttgcctatggccgagtccacccgcgacggcctgggccctggcttgttgtcgcccatcgtgtcccctctgaaaggccttggccccccgccgctgcccccctcttctcagtcccatagtcccggtgggcagccgtttcccaccctgccctctaagccctcctatcccccgttccagagtccacccccccccccactgccgtccccgcagggctaccagggctccttccattctatccagtcttgctttccttacggcgattgttacaggatggctgagcccgcagctgggggcgatgggctggtgggcgagacacatggattcaatccactcaggcccaacggttatcactcgctatccactccgttgcctgccacgggttatgaggcgctggcagaggctagctgcccaacggctctgcctcagcagccatcagaggacgtggtcagcagcggccctgaagactgcggcttctttcctaatggggccttcgaccactgtcttggccacatcccgtccatctacacagacacctga